(19) United States
(12) Patent Application Publication
(10) Pub. No.: US 2015/0210964 A1
(43) Pub. Date: Jul. 30, 2015

(54) Consumer Product Compositions

(71) Applicant: The Procter & Gamble Company, Cincinnati, OH (US)

(72) Inventors: Alan David Willey, Cincinnati, OH (US); Jacob Robert Adams, Cincinnati, OH (US); Kady Lynn Willison, Cincinnati, OH (US); Kenneth Edward Yelm, Hamilton, OH (US); Gregory Mark Bunke, Lawrenceburg, IN (US); William Richard Mueller, Cincinnati, OH (US); Robb Richard Gardner, Cleves, OH (US)

(21) Appl. No.: 14/594,189

(22) Filed: Jan. 12, 2015

Related U.S. Application Data
(60) Provisional application No. 61/930,993, filed on Jan. 24, 2014.

(51) Int. Cl. C11D 3/395; B08B 3/10; A61L 2/08; C11D 3/04; A01N 59/00

(52) U.S. Cl. CPC C11D 3/3951; C11D 3/046; C11D 3/3953; A01N 59/00; A61L 2/088; B08B 3/10

(57) ABSTRACT

Consumer product compositions comprise a water soluble organic photoactivator, an electron acceptor which accepts an electron from the photoactivator when the photoactivator is in a photo-excited state and/or reduced state, and a benefit active precursor which converts into a benefit active agent via electron transfer. The electron acceptor is preferably not covalently linked to the photoactivator. Also disclosed are methods of making a benefit active, comprising exposing the consumer product compositions to light, preferably having a wavelength greater than about 350 nm.

### FIELD OF THE INVENTION

The present invention relates to consumer product compositions that include one or more photoactivators to generate one or more benefit active agents, effective as a bleaching agent, stain remover, or antimicrobial and/or in eliminating biofilm. The present invention also relates to methods for cleaning and/or bleaching surfaces, and for providing a method of disinfecting or sanitizing surfaces and/or removing biofilm.

### BACKGROUND OF THE INVENTION

Cleaning compositions are used throughout the world in people's homes and workplaces. These compositions range from surface cleaners and disinfectants to bleach for removing stains from one's clothes or teeth. However, conventional cleaning and whitening compositions are limited by the standard chemistry which generates the cleaning or whitening attribute of the composition.

Conventional low cost cleaners, such as chlorine bleach (sodium hypochlorite), are limited in their ability to disinfect and sanitize. For example, such systems have limited benefit on biofilms, a complex biological community formed extensively in the natural environment by bacteria.

Another attempt at eliminating biofilm is through the production of chlorine dioxide and other biocidal gases. Specifically, it is known that chlorine dioxide can be generated by mixing a chlorine dioxide precursor, such as a metal chlorite, and an activator component, such as a transition metal or acid. When each of the components are combined the chlorine dioxide precursor and activator component react to form chlorine dioxide. Such reactions are highly volatile and toxic and are, therefore, not desirable for home applications. Furthermore, these components must be sequestered to prevent premature formation of the chlorine dioxide. However, multi-compartment packaging is more expensive and can still allow premature mixing of the components and accidental generation of chlorine dioxide. As such, such systems are undesirable.

Yet another attempt at eliminating biofilm is through the use of a photoactivator to produce chlorine dioxide. Specifically, it is known to use titanium dioxide (TiO₂) and a chlorine dioxide precursor in conjunction with exposure to ultraviolet light to generate chlorine dioxide. However, such processes are undesirable due to the health risks associated with exposure to ultraviolet light, the degradation which can occur to the other components of the cleaning compositions, and the use of an insoluble inorganic photoactivator. In addition, titanium dioxide forms particulates which leave undesirable residue on surfaces and requires additives to suspend in and imparts opaqueness to compositions.

As such, there remains a need for a consumer product composition that includes a water-soluble photoactivator that can enable the generation of one or more benefit active agents effective as a bleaching agent, stain remover, or antimicrobial and/or in eliminating biofilm. There further remains a need for a consumer product composition that includes a water-soluble photoactivator that produces a substantially colorless consumer product composition that is effective as a bleaching agent, stain remover, or antimicrobial and/or in eliminating biofilm and activatable by visible light.

### SUMMARY OF THE INVENTION

The present invention, in one aspect, relates to a consumer product composition comprising a water soluble organic photoactivator, an electron acceptor which accepts an electron from the photoactivator when the photoactivator is in a photo-excited state and/or reduced state, and a benefit active precursor which converts into a benefit active agent via electron transfer. The electron acceptor may or may not be covalently linked to the photoactivator, preferably the electron acceptor is not covalently linked to the photoactivator. The benefit active precursor can be an oxyhalite. The present invention also relates to methods for cleaning and/or bleaching surfaces, and for providing a method of disinfecting or sanitizing surfaces and/or eliminating biofilm with the consumer product composition.

In another aspect, the present invention relates to a solid consumer product composition comprising a water soluble organic photoactivator and an oxyhalite. In this regard, the solid composition is dissolved in aqueous solution containing diatomic oxygen, which serves as the electron acceptor to convert the oxyhalite into a benefit active agent (e.g. chlorine dioxide).

In another aspect, the present invention relates to a method of making a benefit active, comprising exposing a consumer product composition to light (e.g. light generated by a natural or an artificial source), preferably having a wavelength greater than 350 nm. The consumer product composition comprises a water soluble organic photoactivator, an electron acceptor which accepts an electron from the photoactivator when the photoactivator is in a photo-excited state and/or reduced state, and a benefit active precursor which converts into a benefit active agent via electron transfer. The electron acceptor may or may not be covalently linked to the photoactivator, preferably the electron acceptor is not covalently linked to the photoactivator.

The present invention further relates to methods of cleaning surfaces, bleaching stains, disinfecting surfaces, and removing biofilms.

It has now been surprisingly found that providing a consumer product composition according to the present invention enables the generation of one or more benefit active agents effective as a bleaching agent, stain remover, or antimicrobial and/or in eliminating biofilm. It has also now been surprisingly found that providing a consumer product composition of the present invention, can produce a consumer product composition that is effective as a bleaching agent, stain remover, or antimicrobial and/or in eliminating biofilm.

### BRIEF DESCRIPTION OF THE DRAWINGS

FIG. 1 is a schematic representing reactions involving the compositions and methods of the present invention.

### DETAILED DESCRIPTION OF THE INVENTION

The present invention relates to consumer product compositions that include water soluble photoactivators. Furthermore, the present invention also relates to photocatalyzable consumer product compositions comprising a photoactivator, an electron acceptor and a benefit active precursor. Still further, the present invention also relates to methods for cleaning and/or bleaching surfaces, and for providing a method of disinfecting or sanitizing surfaces and/or eliminating biofilm using a photoactivator, an electron acceptor and a benefit active precursor.

### Photoactivator

The water soluble photoactivators of the present invention may comprise a photoactive moiety and a hydrophilic moiety. For purposes of the present invention, the term “hydrophilic moiety” refers to a moiety that is attracted to water and dissolves in water to form a homogenous solution. In one embodiment, the hydrophilic moiety is selected from the group consisting of water soluble oligimers, water soluble polymers and water soluble copolymers. In one preferred embodiment, the hydrophilic moiety may be selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, arginine, histidine, aspartic acid, glutamic acid, serine, threonine, asparagine, glutamine, isopropyl acrylamide, styrene sulfonic acid, vinyl methyl ether, vinyl phosphoinic acid, ethylene imine, and mixtures thereof. In one especially preferred embodiment, the hydrophilic moiety may be selected from the group consisting of alkylene oxide oligimer polymers, alkylene oxide oligimer copolymers, vinyl alcohol, vinyl pyrrolidone, acrylic acid, acrylamide, cellulose, and mixtures thereof. For purposes of the present invention, the term “photoactive moiety” refers to an organic conjugated moiety that is capable of absorbing a photon of light and thereby forming an excited state (singlet or triplet). It will be understood that the term “photoactive moiety” does not, however, refer to a charge-transfer excited state. It will further be understood that the photoactive moieties, as disclosed herein, may include a single moiety or a combination of two, three, four or any other number of moieties, as known in the art.

In one embodiment of the present invention, the photoactive moiety is selected from the group consisting of 1,1′-biphenyl-4,4′-diamine, 1,1′-biphenyl-4-amine, benzophenone, 1,1′-biphenyl-4,4′-diol, 1,1′-biphenyl-4-amine, 1,1′-biphenyl-4-ol, 1,1′:2′,1″-terphenyl, 1,1′:3′,1″-terphenyl, 1,1′:4′,1″:4″,1′″-quaterphenyl, 1,1′:4′,1″-terphenyl, 1,10-phenanthroline, 1,1′-biphenyl, 1,2,3,4-dibenzanthracene, 1,2-benzenedicarbonitrile, 1,3-isobenzofurandione, 1,4-naphthoquinone, 1,5-naphthalenediol, 10H-phenothiazine, 10H-phenoxazine, 10-methylacridone, 1-acetonaphthone, 1-chloroanthraquinone, 1-hydroxyanthraquinone, 1-naphthalenecarbonitrile, 1-naphthalenecarboxaldehyde, 1-naphthalenesulfonic acid, 1-naphthalenol, 2(1H)-quinolinone, 2,2′-biquinoline, 2,3-naphthalenediol, 2,6-dichlorobenzaldehyde, 21H,23H-porphine, 2-aminoanthraquinone, 2-benzoylthiophene, 2-chlorobenzaldehyde, 2-chlorothioxanthone, 2-ethylanthraquinone, 2H-1-benzopyran-2-one, 2-methoxythioxanthone, 2-methyl-1,4-naphthoquinone, 2-methyl-9(10-methyl)-acridinone, 2-methylanthraquinone, 2-methylbenzophenone, 2-naphthalenamine, 2-naphthalenecarboxylic acid, 2-naphthalenol, 2-nitro-9(10-methyl)-acridinone, 9(10-ethyl)-acridinone, 3,6-qcridinediamine, 3,9-dibromoperylene, 3,9-dicyanophenanthrene, 3-benzoylcoumarin, 3-methoxy-9-cyanophenanthrene, 3-methoxythioxanthone, 3′-methylacetophenone, 4,4′-dichlorobenzophenone, 4,4′-dimethoxybenzophenone, 4-bromobenzophenone, 4-chlorobenzophenone, 4′-fluoroacetophenone, 4-methoxybenzophenone, 4′-methylacetophenone, 4-methylbenzaldehyde, 4-methylbenzophenone, 4-phenylbenzophenone, 6-methylchromanone, 7-(diethylamino)coumarin, 7H-benz[de]anthracen-7-one, 7H-benzo[c]xanthen-7-one, 7H-furo[3,2-g][1]benzopyran-7-one, 9(10H)-acridinone, 9(10H)-anthracenone, 9(10-methyl)-acridinone, 9(10-phenyl)-acridinon, 9,10-anthracenedione, 9-acridinamine, 9-cyanophenanthrene, 9-fluorenone, 9H-carbazole, 9H-fluoren-2-amine, 9H-fluorene, 9H-thioxanthen-9-ol, 9H-thioxanthen-9-one, 9H-thioxanthene-2,9-diol, 9H-xanthen-9-one, acetophenone, acridene, acridine, acridone, anthracene, anthraquinone, anthrone, α-tetralone, benz[a]anthracene, benzaldehyde, benzamide, benzo[a]coronene, benzo[a]pyrene, benzo[f]quinoline, benzo[ghi]perylene, benzo[rst]pentaphene, benzophenone, benzoquinone, 2,3,5,6-tetramethyl, chrysene, coronene, dibenz[a,h]anthracene, dibenzo[b,def]chrysene, dibenzo[c,g]phenanthrene, dibenzo[def,mno]chrysene, dibenzo[def,p]chrysene, DL-tryptophan, fluoranthene, fluoren-9-one, fluorenone, isoquinoline, methoxycoumarin, methylacridone, michler's ketone, naphthacene, naphtho[1,2-g]chrysene, N-methylacridone, p-benzoquinone, p-benzoquinone, 2,3,5,6-tetrachloro, pentacene, phenanthrene, phenanthrenequinone, phenanthridine, phenanthro[3,4-c]phenanthrene, phenazine, phenothiazine, p-methoxyacetophenone, pyranthrene, pyrene, quinoline, quinoxaline, riboflavin 5′-(dihydrogen phosphate), thioxanthone, thymidine, xanthen-9-one, xanthone, derivatives thereof, and mixtures thereof.

Preferably, the photoactive moiety is selected from the group consisting of xanthone, xanthene, thioxanthone, thioxanthene, phenothiazine, fluorescein, benzophenone, alloxazine, isoalloxazine, flavin, derivatives thereof, and mixtures thereof. In one preferred embodiment, the photoactive moiety is thioxanthone.

Other suitable water-soluble photoactivators for the consumer product compositions of the present invention include fluoresceins and derivatives thereof; preferably halogen substituted fluoresceins; more preferably bromo- and iodo-fluoresceins such as dibromo fluorescein, diodo fluorescein, rose bengal, erythrosine, eosin (e.g. Eosin Y).

It is a further aspect of the present invention that the photoactivator preferably comprises less than about 35%, about 30%, about 25%, about 20%, about 15%, about 10%, about 5%, about 3% and about 2%, by weight of the photoactivator, of the photoactive moiety. As such, the photoactivator preferably comprises at least about 65%, about 70%, about 75%, about 80%, about 85%, about 90%, about 95%, about 97%, and about 98%, by weight of the photoactivator, of hydrophilic moiety. In one aspect, the photoactivator comprises less than about 2%, by weight of the photoactivator, of photoactive moiety (such as thioxanthone), and at least about 98%, by weight of the photoactivator, of hydrophilic moiety (such as polyethylene glycol). Without wishing to be bound by theory, it is believed that such a photoactivator not only is water soluble, but will resist aggregation due to the steric hindrance imparted by the hydrophilic moiety or any other non-photoactive moiety.

It is still further another aspect of the present invention that the photoactive moiety has an absorption band between about 350 nm and about 750 nm, about 350 nm and about 600 nm, about 350 nm and about 420 nm, and about 380 nm and about 400 nm.

In another embodiment, the photoactive moiety does not have an absorption band between about 420 nm and about 720 nm, about 500 and about 700 nm, about 500 nm and about 650 nm, and about 500 nm and about 600 nm. In this embodiment, it will be understood that the photoactivator will be substantially colorless to the human eye when used in an aqueous solution at a concentration of about 500 ppm.

In yet another aspect of the present invention, the photoactivator can be activated to a photo-excited state by excitation with incident radiation of a wavelength greater than 350 nm, preferably between about 350 nm and about 420 nm. In one embodiment, the photo-excited state lifetime is greater than about 0.5 nanosecond, 1 nanosecond, 10 nanoseconds, 50 nanoseconds, 100 nanoseconds, 300 nanoseconds and 500 nanoseconds. In another embodiment, the photo-excited state of the photoactivator has an energy greater than about 100 kJ/mol, 150 kJ/mol, 200 kJ/mol and 300 kJ/mol more than a ground state of the photoactivator.

In one embodiment, the photoactivator can be excited to a “singlet state” and in another a “triplet state”, as both of those terms are known in the art.

In yet another embodiment, the present invention relates to a photoactivator having the formula:

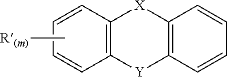

wherein,
X is selected from the group consisting of C, O, NH, C═O, CH₂, CHR″, CR″R′″, S, SO, and SO₂;
Y is selected from the group consisting of C, O, NH, C═O, CH₂, CHR″, CR″R′″, S, SO, and SO₂;
R′, R″ and R′″ may be —H or selected from a group of substituents that include a moiety selected from the group consisting of Oxygen, Nitrogen, Sulfur, Halogen and Hydrocarbon;
at least one of R′, R″ or R′″ further comprises a hydrophilic moiety R;
R is selected from the group consisting of water soluble oligimers, water soluble polymers and water soluble copolymers;
m is an integer from 0-8; and
the combined molecular weight of the substituents R′, R″ and R′″ is greater than 400 atomic mass units (AMU).

It can be appreciated by one of ordinary skill in the art that the substituent(s) R′ as depicted in the formula above reflects that the substitution of the photoactivator may include any number of substituents from zero to eight and that these substituents may be covalently attached to the peripheral carbon atoms of the photoactivator. Where m>1, the multiple R′ groups can be independently selected from a group of substituents that include a moiety selected from the group consisting of Oxygen, Nitrogen, Sulfur, Halogen and Hydrocarbon.

In one embodiment, R may be selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, arginine, histidine, aspartic acid, glutamic acid, serine, threonine, asparagine, glutamine, isopropyl acrylamide, styrene sulfonic acid, vinyl methyl ether, vinyl phosphoinic acid, ethylene imine, and mixtures thereof.

R′, R″ and R′″ moieties that may replace hydrogen and which contain only carbon and hydrogen atoms include any hydrocarbon moieties, as known in the art, including, alkyl, alkenyl, alkynyl, alkyldienyl, cycloalkyl, phenyl, alkyl phenyl, naphthyl, anthryl, phenanthryl, fluoryl, steroid groups, and combinations of these groups with each other and with polyvalent hydrocarbon groups such as alkylene, alkylidene and alkylidyne groups. Specific non-limiting examples of such groups are:

—CH₃, —CHCH₃CH₃, —(CH₂)ₙCH₃, —CH₂—C≡CH, —CH═CH—CH═CH₂,

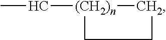

### —φCH₃, —φCH₂φ, -φ, and -φ-φ.

where n is independently chosen as being from 0-22

R′, R″ and R′″ moieties containing oxygen atoms that may replace hydrogen include hydroxy, acyl or keto, ether, epoxy, carboxy, and ester containing groups. Specific non-limiting examples of such oxygen containing groups are:
—CH₂OH, —CCH₃CH₃OH, —CH₂COOH, —C(O)—(CH₂)ₙCH₃, —C(O)—R, —C(O)—OR, —O(CH₂)ₙCH₃, —O—R, ═O, —OH, —(CH₂)ₙ—O—(CH₂)ₙCH₃, —(CH₂)ₙ—O—R, —(CH₂)ₙ—O—(CH₂)ₙ—OH, —(CH₂)ₙCOOH, —(CH₂)ₙCOOR, -φOH, -φO(CH₂)ₙCH₃, φO—R, -φ(CH₂)ₙOH,

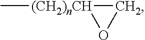

and

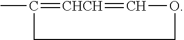

where n is independently chosen as being from 0-22

R′, R″ and R′″ moieties containing sulfur atoms that may replace hydrogen include the sulfur-containing acids and acid ester groups, thioether groups, mercapto groups and thioketo groups. Specific non-limiting examples of such sulfur containing groups are:
—S(CH₂)ₙCH₃, —(CH₂)ₙS(CH₂)ₙCH₃, —SO₃(CH₂)ₙCH₃, SO₂(CH₂)ₙCH₃, —(CH₂)ₙCOSH, —SH, —(CH₂)ₙSCO, —(CH₂)ₙC(S)(CH₂)ₙCH₃, —SO₃H, —O(CH₂)ₙC(S)CH₃, —S—R, —(CH₂)ₙS—R, —SO₃—R, SO₂—R, —(CH₂)ₙCOS—R, —(CH₂)ₙC(S)—R, —O(CH₂)ₙC(S)—R, ═S, and

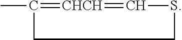

where n is independently chosen as being from 0-22

R′, R″ and R′″ moieties containing nitrogen atoms that may replace hydrogen include amino groups, the nitro group, azo groups, ammonium groups, amide groups, azido groups, isocyanate groups, cyano groups and nitrile groups. Specific non-limiting examples of such nitrogen containing groups are:
—NH₂, —NH₃⁺, —NH(CH₂)ₙCH₃, —N((CH₂)ₙCH₃)₂, —(CH₂)ₙNH(CH₂)ₙCH₃, (CH₂)ₙN((CH₂)ₙCH₃)₂, —CH₂CONH₂, —CH₂CONH(CH₂)ₙCH₃, —CH₂CON((CH₂)ₙCH₃)₂, —NRH₂⁺, —NH—R, —NR₂, —(CH₂)ₙNH—R, —(CH₂)ₙNR₂, —(CH₂)ₙCONH—R, —(CH₂)ₙCONR₂, —(CH₂)ₙCON₃, —(CH₂)ₙCH═NOH, —CN, —CH(CH₂)ₙNCO, —(CH₂)ₙNCO, —NO, -φN═NOH, and ≡N.

where n is independently chosen as being from 0-22.

R′, R″ and R′″ moieties containing halogen atoms that may replace hydrogen include chloro, bromo, fluoro, iodo groups and any of the moieties previously described where a hydrogen or a pendant alkyl group is substituted by a halo group to form a stable substituted moiety. Specific non-limiting examples of such halogen containing groups are: —Cl, —Br, —I, —(CH₂)ₙCOCl, -φF₅, -φCl, —CF₃, and —(CH₂)ₙφBr.

It is understood that any of the above moieties that may replace hydrogen can be substituted into each other in either a monovalent substitution or by loss of hydrogen in a polyvalent substitution to form another monovalent moiety that can replace hydrogen in the organic compound or radical.

As used herein “φ” represents a phenyl ring.

### Photocatalyzable Consumer Product Composition

The present invention also relates to photocatalyzable consumer product compositions that include the photoactivator, as described in further detail above, an electron acceptor and a benefit active precursor. As used herein, consumer product compositions encompass beauty care compositions, fabric and home care compositions, and health care compositions. Beauty care compositions generally include compositions for treating hair, including, bleaching, coloring, dyeing, conditioning, growing, removing, retarding growth, shampooing, styling; deodorants and antiperspirants; personal cleansing; color cosmetics; products, and/or methods relating to treating skin, including application of creams, lotions, and other topically applied products for consumer use; and products and/or methods relating to orally administered materials for enhancing the appearance of hair, skin, and/or nails; and shaving. Fabric and home care compositions generally include compositions for treating fabrics, hard surfaces and any other surfaces in the area of fabric and home care, such as car care, dishwashing, fabric conditioning (including softening), laundry detergency, laundry and rinse additive and/or care, hard surface cleaning and/or treatment, and other cleaning for consumer or institutional use. Oral care compositions generally include compositions for use with any soft and/or hard tissue of the oral cavity or conditions associated therewith, e.g., anti-caries compositions, anti-microbial compositions, anti-plaque chewing gum, compositions, breath compositions, confectionaries, dentifrices/toothpastes, denture compositions, lozenges, rinses, and tooth whitening compositions.

The photocatalyzable consumer product composition may be an aqueous solution, a solid, or incorporated into a material, such as a film. In another embodiment, the individual components of the photocatalyzable consumer product composition may be incorporated into both an aqueous solution and a material, such as a film. In one embodiment, the photoactivator may be included in a film and the electron acceptor and/or benefit active precursor maybe included in an aqueous solution. It will be understood that in this particular embodiment, a film comprising a photoactivator may be applied to surface and an aqueous solution comprising an electron acceptor and benefit active precursor may be applied separately.

However, if the photocatalyzable consumer product composition is an aqueous composition, the composition may comprise from 1% to 99%, by weight of the composition, of water. It will therefore be understood that the photocatalyzable consumer product composition can be in concentrated or diluted form. It is further contemplated that all or a portion of the water may be replaced with another solvent such as ethanol, glycol, glycol-ethers, glycerin, water soluble acetates and alcohols.

As noted above, the present invention relates to photocatalyzable consumer product compositions that include the photoactivator, an electron acceptor and a benefit active precursor. In such embodiments it will be understood that the photocatalyst can be excited into a singlet and/or triplet state via activation by light in the visible wavelengths. It will also be understood that the benefit active precursor can be converted into a benefit active agent upon triggering by the photocatalyst in an activated singlet and/or triplet state after exposure to visible light. It will be understood that the photocatalyst is unreactive with the benefit active precursor without activation by light.

The photocatalyzable consumer product composition is a system responsive to light; for example, visible, ultraviolet and/or infrared. In one preferred embodiment, the system is responsive visible light. In the present embodiment, photon transfer from the light source to the photocatalyst allows the reaction to progress to create an effective benefit agent that, in some embodiments, may act to clean, disinfect or sanitize, and/or bleach or whiten.

### Electron Acceptor

The photocatalyzable consumer product composition of the present invention comprises an electron acceptor. It will be understood to those skilled in the art that photocatalytic reduction and oxidation chemistries differ from conventional, energy-transfer photochemistry in that the photocatalytically-induced transfer of electrons can result in chemical transformation of reagents (e.g. transformation of the benefit precursor material to the benefit active) and oxidation of the benefit precursor material to produce a benefit active which is capable of providing a beneficial result, for example, cleaning, disinfection, bleaching, and/or whitening.

For the purposes of the present invention the term “electron acceptor” is defined as “a compound or moiety which accepts an electron from the photoactivator when the photoactivator is in a photo-excited state and/or one electron reduced state.” This electron transfer process is normally a very rapid and reversible process.

The ability of the electron acceptor to accept an electron from the excited photoactivator is generally described in Turro, N. J., V. Ramamurthy, and J. C. Scaiano, Principles of Molecular Photochemistry: An Introduction, Chapter 7, p. 41 (University Science Books 2009, Paperback edition). It is understood that the reaction between the reactants is favored when the Gibbs free energy (delta G) is less than 0.

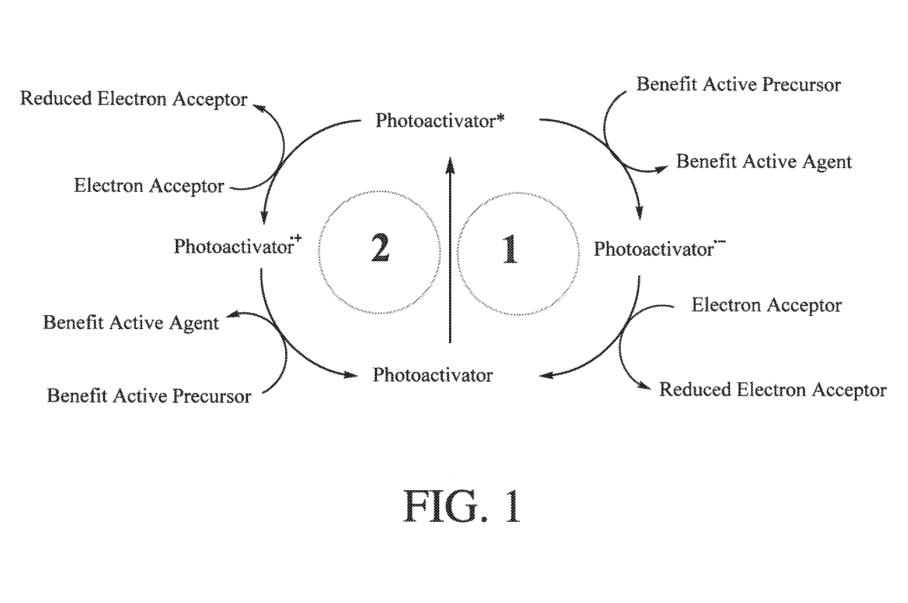

The reaction process is exemplified schematically in FIG. 1. As shown in FIG. 1, Reaction 1 (the right half of the FIGURE) illustrates a reaction in which electron transfer occurs from a benefit acative precursor to the excited state of the photoactivator (thereby forming a benefit active) and then from the one-electron reduced form of the photoactivator to the electron acceptor as described herein. As shown in FIG. 1, Reaction 2 (the left half of the FIGURE) illustrates a reaction in which electron transfer occurs from the excited state of the photoactivator to the electron acceptor and then from the one electron oxidized form of the photoactivator to the benefit active precursor (thereby forming a benefit active). In all cases, the Gibbs free energy for the electron transfer should be less than 0. It is understood that the conversion of the photoactivator to its photoactivated state (“Photoactivator*”) is initiated by the absorption of light, which is also present in the reaction.

It will further be understood to those skilled in the art that any electron transfer between species comprising the photocatalyzable consumer product composition further requires effective Brownian collision to occur between the reacting species and that effective electron transfer between the photochemically excited state of the photoactivator and any species comprising the photocatalyzable consumer product composition (e.g. the electron acceptor) may further depend on the lifetime of the excited state of the photoactivator, the concentration of the photoactivator, and the concentration of the electron acceptor.

The electron acceptor of the present invention may be any species that accepts an electron from the photoactivator when the photoactivator is in a photo-excited state and/or reduced state. The electron acceptor must be present in the photocatalyzable consumer product composition in sufficient concentration to enable Brownian collisions with the photoactivator, given the concentration of the photoactivator and the lifetime of the photochemically excited state of the photoactivator.

A suitable electron acceptor acceptor can be selected from the group consisting of:

Viologens: e.g., methyl viologen;

Biyridiums: e.g., 2,2′ bipyridinium, 3,3′ bipyridinium, 3,4′ bipyridinium;

Quinones: e.g., para-Benzoquinone, 2,3-Dichloro-5,6-dicyano-p-benzoquinone, Tetrahydroxy-1,4-quinone hydrate, 2,5-di-tert-butylhydroquinone, tert-Butylhydroquinone, Anthraquinone, Diaminoanthroquinone, Anthraquinone-2-sulfonic acid;

Polycyclic aromatic hydrocarbons: e.g., Naphthalene, Anthracene, Pyrene, Dicyanobenzene, dicyano naphthalene, dicyano anthracene, dicyanopyrene;

Transition metal salts: e.g., Chloropentaamine cobalt dichloride, Silver nitrate, Iron Sulfate, copper sulfate;

Nanoparticle semiconductors: e.g., Titanium Dioxide, Zinc Oxide, Cadmium Selenide;

Persulfates: e.g., Ammonium persulfate, Sodium persulfate, Potassium persulfate;

Nitroxyl radicals: e.g., (2,2,6,6-Tetramethylpiperidin-1-yl)oxy, Dimethylthiourea, Tetranitromethane, Lithium, sodium and potassium acetoacetate, Oxaloacetic acid;

Ascorbic acid salts: e.g., Sodium ascorbate;

Phenols: 2,6-Dicholorophenolindophenol, 4-methoxyphenol;

Others: 4-Methylmorpholine N-oxide, 4-tert-Butylcatechol, Allopurinol, Pyridoxal 5′-phosphate, pyridoxal hydrochloride, Sodium benzoate, Sodium Nitrate, Sodium Nitrite, Diatomic Oxygen; and

Mixtures thereof.

With respect to suitable electron acceptors, diatomic oxygen is an electron acceptor which can be present in the composition due to dissolution of oxygen from the atmosphere into the composition, especially in an aqueous liquid composition. Most aqueous liquid compositions will have a sufficient content of diatomic oxygen as an electron acceptor to enable the electron transfer process. This can be enhanced with the addition of other electron acceptors in the composition as an ingredient. With respect to solid compositions (or other substantially anhydrous compositions), such compositions typically will not have a sufficient level of diatomic oxygen to enable the electron transfer process. Therefore, a solid composition which does not contain an electron acceptor as an added ingredient to the composition can nonetheless be photochemically active upon dissolution of the solid composition into an aqueous solution due to the presence of diatomic oxygen in the aqueous solution (e.g. a solid detergent composition that is dissolved in water can form an aqueous solution containing diatomic oxygen at a level sufficient to enable the electron transfer process). The present invention therefore encompasses a solid composition comprising a water soluble photoactivator and an oxyhalite, without an electron acceptor being added to the composition as an ingredient. Such a solid composition can be photoactivated upon dissolution in water wherein diatomic oxygen can serve as the electron acceptor.

With respect to suitable electron acceptors, nanoparticle semiconductors such as titanium dioxide can be used at relatively low levels to serve as electron acceptors, preferably less than about 1%, preferably less than 0.5%, preferably less than 0.1%, preferably less than 0.05%, preferably less than 0.01%, by weight of the consumer product composition. At higher levels, such materials may function efficiently as photoactivators, however any use of nanoparticle semiconductors in the present invention is preferably at a low enough level such that the material does not function efficiently as a photoactivator to provide significant consumer noticeable benefits and functions instead as an electron acceptor.

The photocatalyzable consumer product composition is preferably an aqueous composition and the electron acceptor is preferably a water soluble species selected from one or more of the groups listed above.

### Benefit Active Precursor

The photocatalyzable consumer product composition of the present invention comprises a benefit active precursor. When used in the photocatalyzable consumer product composition of the present invention and exposed to appropriate light (such as in the methods of the present invention), the benefit active precursor is converted into a benefit active (such as chlorine dioxide). The benefit active is the one electron oxidation product(s) of the benefit active precursor.

In one aspect of the present invention, the benefit active precursor is a material selected from one or more species according to the following formula:

A[XOₙ]ₘ

wherein
A is selected from the group consisting of monovalent cations, divalent cations, and trivalent cations; A can be an organic or inorganic cation; A is preferably selected from the group consisting of Aluminum, Barium, Calcium, Cobalt, Chromium, Copper, Iron, Lithium, Potassium, Rubidium, Magnesium, Manganese, Molybdenum, Nickel, Sodium, Titanium, Vanadium, Zinc, ammonium, alkyl-ammonium, aryl-ammonium, and mixtures thereof; A is more preferably selected from the group consisting of lithium, sodium, potassium, magnesium, calcium, ammonium, and mixtures thereof;
X is selected from the group consisting of chlorine, bromine, iodine, and mixtures thereof;
n is 1, 2, 3, or 4, preferably n is 2, 3, or 4; and
m is 1, 2, or 3.

The benefit active precursor of the present invention is preferably an oxyhalite, and is preferably selected from the group consisting of hypochlorite salts, chlorite salts, chlorate salts, perchlorate salts, hypobromite salts, bromite salts, bromate salts, perbromate salts, hypoiodate salts, iodite salts, iodate salts, periodate salts and mixtures thereof. Suitable benefit active precursors include those selected from the group consisting of sodium chlorite, sodium bromite, sodium iodite, potassium chlorite, potassium bromite, potassium iodite, sodium chlorate, sodium bromate, sodium iodate, potassium chlorate, potassium bromate, potassium iodate, sodium hypochlorite, sodium hypobromite, sodium hypoiodite, sodium perchlorate, potassium perchlorate, and mixtures thereof. In at least one aspect, the benefit active precursor is not a hypo-halite, such as hypochlorite.

In one aspect, the benefit active precursor may be a chlorite salt. A specific example of a chlorite salt suitable for use as a benefit active precursor is sodium chlorite (NaClO₂). In this embodiment, activation of the chlorite salt through transfer of an electron to the photoactivated photocatalyst results in the formation of the benefit active chlorine dioxide (ClO₂). Chlorine dioxide is a potent biocide and bleaching agent. In addition to salts, various other precursor forms are contemplated herein.

### Optional Additives

The photocatalyzable consumer product compositions of the invention may also contain additional adjunct additives. The precise nature of these additional components and levels of incorporation thereof will depend on the physical form of the composition, and the precise nature of the cleaning, disinfecting and/or whitening operation for which it is to be used. It will be understood that some of the adjunct additives noted below will have photoactive and/or electron acceptor properties, but it will be further understood that such additives will not replace the components noted above.

### Fabric Care Additives

Adjunct cleaning additives may be selected from the group consisting of nonionic surfactants, cationic surfactants, zwitterionic or amphoteric surfactants, builders, structurants or thickeners, clay soil removal/anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, mica, fabric hueing agents, dye transfer inhibiting agents, chelating agents, suds suppressors/anti-foams, fabric softeners, and perfumes, as well as such solvents, stabilizers, antimicrobial agents, and neutralizers required to formulate such product.

Compounds for reducing or suppressing the formation of suds can be incorporated into the photocatalyzable compositions of the present invention. Suds suppression can be of particular importance in the so-called “high concentration cleaning process” as described in U.S. Pat. Nos. 4,489,455 and 4,489,574, and in front-loading-style washing machines.

To minimize or eliminate residue in the laundering equipment an anti-foam or anti-suds agent is beneficial. Anti-foam, anti-suds agent, and suds suppressor are interchangeable names for the same functional additive or additives. A wide variety of materials may be used as suds suppressors, and suds suppressors are well known to those skilled in the art. See, for example, Kirk Othmer Encyclopedia of Chemical Technology, Third Edition, Volume 7, pages 430-447 (John Wiley & Sons, Inc., 1979). Examples of suds suppressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100° C., silicone suds suppressors, and secondary alcohols. Suds suppressors are described in U.S. Pat. Nos. 2,954,347; 4,265,779; 4,265,779; 3,455,839; 3,933,672; 4,652,392; 4,978,471; 4,983,316; 5,288,431; 4,639,489; 4,749,740; and 4,798,679; 4,075,118; European Patent Application No. 89307851.9; EP 150,872; and DOS 2,124,526.

When incorporated as an adjunct ingredient, suds suppressors are generally incorporated from 0% to about 10%, by weight. When utilized as suds suppressors, monocarboxylic fatty acids, and salts therein, will be present typically in amounts up to about 5%, by weight, of the photocatalyzable consumer product composition. Preferably, from about 0.5% to about 3% of fatty monocarboxylate suds suppressor is utilized. Silicone suds suppressors are typically utilized in amounts up to about 2.0%, by weight, of the detergent composition, although higher amounts may be used. Monostearyl phosphate suds suppressors are generally utilized in amounts ranging from about 0.1% to about 2%, by weight, of the composition. Hydrocarbon suds suppressors are typically utilized in amounts ranging from about 0.01% to about 5.0%, although higher levels can be used. The alcohol suds suppressors are typically used at 0.2%-3% by weight of the finished compositions.

Perfumes and perfumery ingredients useful in the present photocatalyzable consumer product compositions comprise a wide variety of natural and synthetic chemical ingredients, including, but not limited to, hydrocarbons, alcohols, aldehydes, ketones, esters, and the like. Also included are various natural extracts and essences which can comprise complex mixtures of ingredients, such as orange oil, lemon oil, rose extract, lavender, musk, patchouli, balsamic essence, sandalwood oil, pine oil, cedar, and the like. Finished perfumes can comprise extremely complex mixtures of such ingredients. Finished perfumes typically comprise from about 0.01% to about 2%, by weight, of the photocatalyzable compositions herein, and individual perfumery ingredients can comprise from about 0.0001% to about 90% of a finished perfume composition.

When combined with a detergent composition, the photocatalyzable cleaning compositions of the present invention may optionally comprise a builder. Built detergents typically comprise at least about 1 wt % builder, based on the total weight of the detergent. Liquid formulations typically comprise up to about 10 wt %, more typically up to 8 wt % of builder to the total weight of the detergent.

Detergent builders, when used, are typically silicates, to assist in controlling mineral, especially calcium and/or magnesium, hardness in wash water or to assist in the removal of particulate soils from surfaces. Suitable builders can be selected from the group consisting of phosphates and polyphosphates, especially the sodium salts; carbonates, bicarbonates, sesquicarbonates and carbonate minerals other than sodium carbonate or sesquicarbonate; organic mono-, di-, tri-, and tetracarboxylates especially water-soluble nonsurfactant carboxylates in acid, sodium, potassium or alkanolammonium salt form, as well as oligomeric or water-soluble low molecular weight polymer carboxylates including aliphatic and aromatic types; and phytic acid. These may be complemented by borates, e.g., for pH-buffering purposes, or by sulfates, especially sodium sulfate and any other fillers or carriers which may be important to the engineering of stable surfactant and/or builder-containing detergent compositions. Other detergent builders can be selected from the polycarboxylate builders, for example, copolymers of acrylic acid, copolymers of acrylic acid and maleic acid, and copolymers of acrylic acid and/or maleic acid and other suitable ethylenic monomers with various types of additional functionalities. Also suitable for use as builders herein are synthesized crystalline ion exchange materials or hydrates thereof having chain structure and a composition represented by the following general Formula I an anhydride form: x(M₂O).ySiO₂.zM′O wherein M is Na and/or K, M′ is Ca and/or Mg; y/x is 0.5 to 2.0 and z/x is 0.005 to 1.0 as taught in U.S. Pat. No. 5,427,711.

The photocatalytic consumer product compositions may be structured liquids. Structured liquids can either be internally structured, whereby the structure is formed by primary ingredients (e.g. surfactant material) and/or externally structured by providing a three dimensional matrix structure using secondary ingredients (e.g. polymers, clay and/or silicate material). The photocatalyzable consumer product composition may comprise a structurant, preferably from 0.01 wt % to 5 wt %, from 0.1 wt % to 2.0 wt % structurant. The structurant is typically selected from the group consisting of diglycerides and triglycerides, ethylene glycol distearate, microcrystalline cellulose, cellulose-based materials, microfiber cellulose, biopolymers, xanthan gum, gellan gum, and mixtures thereof. A suitable structurant includes hydrogenated castor oil, and non-ethoxylated derivatives thereof. A suitable structurant is disclosed in U.S. Pat. No. 6,855,680. Such structurants have a thread-like structuring system having a range of aspect ratios. Other suitable structurants and the processes for making them are described in WO2010/034736.

The photocatalyzable consumer product compositions of the present invention may contain water-soluble ethoxylated amines having clay soil removal and antiredeposition properties. Exemplary clay soil removal and antiredeposition agents are described in U.S. Pat. Nos. 4,597,898; 548,744; 4,891,160; European Patent Application Nos. 111,965; 111,984; 112,592; and WO 95/32272.

Polymeric soil release agents, hereinafter “SRA” or “SRA's”, can optionally be employed in the present fabric treatment when combined with a detergent composition. If utilized, SRA's will generally comprise from 0.01% to 10.0%, typically from 0.1% to 5%, preferably from 0.2% to 3.0% by weight, of the photocatalyzable consumer product composition.

Preferred SRA's typically have hydrophilic segments to hydrophilize the surface of hydrophobic fibers such as polyester and nylon, and hydrophobic segments to deposit upon hydrophobic fibers and remain adhered thereto through completion of washing and rinsing cycles thereby serving as an anchor for the hydrophilic segments. This can enable stains occurring subsequent to treatment with SRA to be more easily cleaned in later washing procedures.

SRA's can include, for example, a variety of charged, e.g., anionic or even cationic (see U.S. Pat. No. 4,956,447), as well as noncharged monomer units and structures may be linear, branched or even star-shaped. They may include capping moieties which are especially effective in controlling molecular weight or altering the physical or surface-active properties. Structures and charge distributions may be tailored for application to different fiber or textile types and for varied detergent or detergent additive products. Examples of SRAs are described in U.S. Pat. Nos. 4,968,451; 4,711,730; 4,721,580; 4,702,857; 4,877,896; 3,959,230; 3,893,929; 4,000,093; 5,415,807; 4,201,824; 4,240,918; 4,525,524; 4,201,824; 4,579,681; and 4,787,989; European Patent Application 0 219 048; 279,134 A; 457,205 A; and DE 2,335,044.

Polymeric dispersing agents can advantageously be utilized at levels from about 0.1% to about 7%, by weight, in the photocatalyzable consumer product compositions herein, especially in the presence of zeolite and/or layered silicate builders. Suitable polymeric dispersing agents include polymeric polycarboxylates and polyethylene glycols, although others known in the art can also be used. For example, a wide variety of modified or unmodified polyacrylates, polyacrylate/maleates, or polyacrylate/methacrylates are highly useful. Examples of polymeric dispersing agents are found in U.S. Pat. No. 3,308,067, European Patent Application No. 66915, EP 193,360, and EP 193,360.

Soil suspension, grease cleaning, and particulate cleaning polymers may include the alkoxylated polyamines. Such materials include but are not limited to ethoxylated polyethyleneimine, ethoxylated hexamethylene diamine, and sulfated versions thereof. Polypropoxylated derivatives are also included. A wide variety of amines and polyalklyeneimines can be alkoxylated to various degrees, and optionally further modified to provide the abovementioned benefits. A useful example is 600 g/mol polyethyleneimine core ethoxylated to 20 EO groups per NH and is available from BASF.

Alkoxylated polycarboxylates such as those prepared from polyacrylates are useful herein to provide additional grease removal performance. Such materials are described in WO 91/08281 and PCT 90/01815. Chemically, these materials comprise polyacrylates having one ethoxy side-chain per every 7-8 acrylate units. The side-chains are of the formula —(CH₂CH₂O)ₘ (CH₂)ₙCH₃ wherein m is 2-3 and n is 6-12. The side-chains are ester-linked to the polyacrylate “backbone” to provide a “comb” polymer type structure. The molecular weight can vary, but is typically in the range of about 2000 to about 50,000. Such alkoxylated polycarboxylates can comprise from about 0.05% to about 10%, by weight, of the photocatalyzable compositions herein.

Enzymes, including proteases, amylases, other carbohydrases, lipases, oxidases, and cellulases may be used as adjunct ingredients. Enzymes are included in the present photocatalyzable compositions for a variety of purposes, including removal of protein-based, carbohydrate-based, or triglyceride-based stains from substrates, for the prevention of refugee dye transfer in fabric laundering, and for fabric restoration. Preferred selections are influenced by factors such as pH-activity and/or stability optima, thermostability, and stability to active detergents, builders and the like. In one or more embodiments, the compositions may comprise from 0% to 5%, or from about 0.01%-1% by weight of enzyme.

A range of enzyme materials and means for their incorporation into synthetic detergent compositions is also disclosed in WO 9307263 A; WO 9307260 A; WO 8908694 A; U.S. Pat. Nos. 3,553,139; 4,101,457; and U.S. Pat. No. 4,507,219. Enzyme materials useful for liquid detergent formulations, and their incorporation into such formulations, are disclosed in U.S. Pat. No. 4,261,868. Enzymes for use in detergents can be stabilized by various techniques. Enzyme stabilization techniques are disclosed and exemplified in U.S. Pat. Nos. 3,600,319 and 3,519,570; EP 199,405, EP 200,586; and WO 9401532 A.

The enzyme-containing photocatalyzable consumer product compositions herein may optionally also comprise from about 0.001% to about 10%, preferably from about 0.005% to about 8%, most preferably from about 0.01% to about 6%, by weight of an enzyme stabilizing system. The enzyme stabilizing system can be any stabilizing system which is compatible with the detersive enzyme. Such a system may be inherently provided by other formulation actives, or be added separately, e.g., by the formulator or by a manufacturer of detergent-ready enzymes. Such stabilizing systems can, for example, comprise calcium ion, boric acid, propylene glycol, short chain carboxylic acids, boronic acids, and mixtures thereof, and are designed to address different stabilization problems depending on the type and physical form of the photocatalyzable consumer product composition.

When combined with a detergent composition, the photocatalyzable consumer product compositions herein may further contain non-photoactivated bleaching agents or bleaching compositions containing a bleaching agent and one or more non-photoactivated bleach activators.

Examples of non-photoactivated bleaching agents include oxygen bleach, perborate bleach, percarboxylic acid bleach and salts thereof, peroxygen bleach, persulfate bleach, percarbonate bleach, and mixtures thereof. Examples of bleaching agents are disclosed in U.S. Pat. No. 4,483,781, U.S. patent application Ser. No. 740,446, European Patent Application 0,133,354, U.S. Pat. No. 4,412,934, and U.S. Pat. No. 4,634,551.

Examples of non-photoactivated bleach activators (e.g., acyl lactam activators) are disclosed in U.S. Pat. Nos. 4,915,854; 4,412,934; 4,634,551; 4,634,551; and 4,966,723.

Non-photoactivated bleaching agents other than oxygen bleaching agents are also known in the art and can be utilized herein such as pre-formed organic peracids, such as peroxycarboxylic acid or salt thereof, or a peroxysulphonic acid or salt thereof. A suitable organic peracid is phthaloylimidoperoxycaproic acid. If used, household consumer product compositions will typically contain from about 0.025% to about 1.25%, by weight, of such bleaches, especially sulfonate zinc phthalocyanine.

Any optical brighteners or other non-photoactivated brightening or whitening agents known in the art can be incorporated at levels typically from about 0.01% to about 1.2%, by weight, into the photocatalyzable consumer product compositions herein when combined with a consumer product composition. Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982). Specific nonlimiting examples of optical brighteners which are useful in the present compositions are those identified in U.S. Pat. No. 4,790,856 and U.S. Pat. No. 3,646,015.

The photocatalyzable consumer product compositions of the present invention my include fabric hueing agents. Non-limiting examples include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof. In another aspect, suitable polymeric dyes include polymeric dyes selected from the group consisting of fabric-substantive colorants sold under the name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® (Milliken, Spartanburg, S.C., USA) Violet CT, carboxymethyl cellulose (CMC) conjugated with a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colorants, alkoxylated thiophene polymeric colourants, and mixtures thereof. Non-limiting examples of useful hueing dyes include those found in U.S. Pat. No. 7,205,269; U.S. Pat. No. 7,208,459; and U.S. Pat. No. 7,674,757 B2. For example, hueing dye may be selected from the group of: triarylmethane blue and violet basic dyes, methine blue and violet basic dyes, anthraquinone blue and violet basic dyes, azo dyes basic blue 16, basic blue 65, basic blue 66 basic blue 67, basic blue 71, basic blue 159, basic violet 19, basic violet 35, basic violet 38, basic violet 48, oxazine dyes, basic blue 3, basic blue 75, basic blue 95, basic blue 122, basic blue 124, basic blue 141, Nile blue A and xanthene dye basic violet 10, an alkoxylated triphenylmethane polymeric colorant; an alkoxylated thiopene polymeric colorant; thiazolium dye; and mixtures thereof. Preferred hueing dyes include the whitening agents found in WO 08/87497 A1 and those described in US 2008 34511 A1 (Unilever). A preferred agent is “Violet 13”.

The photocatalyzable consumer product compositions of the present invention may also include one or more materials effective for inhibiting the transfer of dyes from one fabric to another during the cleaning process. Generally, such dye transfer inhibiting agents include polyvinyl pyrrolidone polymers, polyamine N-oxide polymers, copolymers of N-vinylpyrrolidone and N-vinylimidazole, manganese phthalocyanine, peroxidases, and mixtures thereof. If used, these agents typically comprise from about 0.01% to about 10% by weight of the composition, preferably from about 0.01% to about 5%, and more preferably from about 0.05% to about 2%. When incorporated in a detergent composition, the photocatalyzable compositions herein may also optionally contain one or more iron and/or manganese and/or other metal ion chelating agents. Such chelating agents can be selected from the group consisting of amino carboxylates, amino phosphonates, polyfunctionally-substituted aromatic chelating agents and mixtures therein. If utilized, these chelating agents will generally comprise from about 0.1% to about 15% by weight of the detergent compositions herein. More preferably, if utilized, the chelating agents will comprise from about 0.1% to about 3.0% by weight of such compositions. The chelant or combination of chelants may be chosen by one skilled in the art to provide for heavy metal (e.g. Fe) sequestration without negatively impacting enzyme stability through the excessive binding of calcium ions. Non-limiting examples of chelants of use in the present invention are found in U.S. Pat. Nos. 7,445,644, 7,585,376 and 2009/0176684 A1.

Useful chelants include heavy metal chelating agents, such as diethylenetriaminepentaacetic acid (DTPA) and/or a catechol including, but not limited to, tiron. In embodiments in which a dual chelant system is used, the chelants may be DTPA and tiron. Other chelating agents suitable for use herein can be selected from the group consisting of aminocarboxylates, aminophosphonates, polyfunctionally-substituted aromatic chelating agents and mixtures thereof. Chelants particularly of use include, but are not limited to: HEDP (hydroxyethanedimethylenephosphonic acid); MGDA (methylglycinediacetic acid); and mixtures thereof.

Aminocarboxylates useful as chelating agents include, but are not limited to, ethylenediaminetetracetates, N-(hydroxyethyl)ethylenediaminetriacetates, nitrilotriacetates, ethylenediamine tetraproprionates, triethylenetetraaminehexacetates, diethylenetriamine-pentaacetates, and ethanoldiglycines, alkali metal, ammonium, and substituted ammonium salts thereof and mixtures thereof. Aminophosphonates are also suitable for use as chelating agents in the compositions of the invention when at least low levels of total phosphorus are permitted in detergent compositions, and include ethylenediaminetetrakis (methylenephosphonates). Preferably, these aminophosphonates do not contain alkyl or alkenyl groups with more than about 6 carbon atoms. Polyfunctionally-substituted aromatic chelating agents are also useful in the compositions herein. See U.S. Pat. No. 3,812,044, issued May 21, 1974, to Connor et al. Preferred compounds of this type in acid form are dihydroxydisulfobenzenes such as 1,2-dihydroxy-3,5-disulfobenzene.

A biodegradable chelator for use herein is ethylenediamine disuccinate (“EDDS”), especially (but not limited to) the [S,S]isomer as described in U.S. Pat. No. 4,704,233. The trisodium salt is preferred though other forms, such as magnesium salts, may also be useful. The chelant system may be present in the fabric treatment compositions of the present invention at from about 0.2% to about 0.7% or from about 0.3% to about 0.6% by weight of the fabric treatment compositions disclosed herein.

Various through-the-wash fabric softeners, especially the impalpable smectite clays of U.S. Pat. No. 4,062,647, as well as other softener clays known in the art, can optionally be used typically at levels of from about 0.5% to about 10% by weight in the present compositions to provide fabric softener benefits concurrently with fabric cleaning. Clay softeners can be used in combination with amine and cationic softeners as disclosed, for example, in U.S. Pat. No. 4,375,416, and U.S. Pat. No. 4,291,071. Cationic softeners can also be used without clay softeners.

Additionally, the photocatalyzable consumer product compositions may optionally include mixtures of nonionic surfactant and anionic surfactant. For the purposes of this invention nonionic surfactants may be defined as substances with molecular structures consisting of a hydrophilic and hydrophobic part. The hydrophobic part consists of a hydrocarbon and the hydrophilic part of a strongly polar group. The nonionic surfactants of this invention are soluble in water. The most preferred nonionic surfactants are alkoxylated, preferably ethoxylated, compounds and carbohydrate compounds. Examples of suitable ethoxylated surfactants include ethoxylated alcohols, ethoxylated alkyl phenols, ethoxylated fatty amides, and ethoxylated fatty esters. Preferred nonionic ethoxylated surfactants have an HLB of from about 10 to about 20. It is advantageous if the surfactant alkyl group contains at least 12 carbon atoms.

Examples of suitable carbohydrate surfactants or other polyhydroxy surfactants include alkyl polyglycosides as disclosed in EP 199 765A and EP 238 638A, polyhydroxy amides as disclosed in WO 93/18125A and WO 92/06161A, fatty acid sugar esters (sucrose esters), sorbitan ester ethoxylates, and poly glycerol esters and alkyl lactobionamides. Preferred nonionic surfactants are these having a long alkyl chain (C 12-C 22) and ethoxylated with 10 to 25 moles of ethylene oxide. Especially preferred nonionic surfactants include tallow alcohol ethoxylated with 15 or 20 moles of ethylene oxide and coco alcohol ethoxylated with 15 or 20 moles of ethylene oxide. Preferred viscosities are achieved when the ratio of polymeric nonionic surfactant to long chain nonionic surfactant is from 10:1 to 1:50, more preferably 5:1 to 1:30, most preferably 3:1 to 1:3

Additionally, the photocatalyzable compositions may optionally include cationic surfactant. Non-limiting examples of cationic surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Additionally, the photocatalyzable consumer product compositions may optionally include amphoteric or zwitterionic surfactant. Non-limiting examples of zwitterionic or ampholytic surfactants include: derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 at column 19, line 38 through column 22, line 48, for examples of zwitterionic surfactants; betaines, including alkyl dimethyl betaine and cocodimethyl amidopropyl betaine, C₈ to C₁₈ (for example from C₁₂ to C₁₈) amine oxides and sulfo and hydroxy betaines, such as N-alkyl-N,N-dimethylamino-1-propane sulfonate where the alkyl group can be C₈ to C₁₈ and in certain embodiments from C₁₀ to C₁₄. Non-limiting examples of ampholytic surfactants include: aliphatic derivatives of secondary or tertiary amines, or aliphatic derivatives of heterocyclic secondary and tertiary amines in which the aliphatic radical can be straight- or branched-chain. One of the aliphatic substituents may contain at least about 8 carbon atoms, for example from about 8 to about 18 carbon atoms, and at least one contains an anionic water-solubilizing group, e.g. carboxy, sulfonate, sulfate. See U.S. Pat. No. 3,929,678 at column 19, lines 18-35, for suitable examples of ampholytic surfactants.

### Oral Care Additives

It would be appreciated by one of ordinary skill in the art that many of the adjunct additives useful in fabric care products and oral care products may generally fall into similar classes, though the specific preferred materials may differ.

The photocatalyzable consumer product composition may be an oral care composition to be topically applied to the mucosal tissue of the oral cavity, to the gingival tissue of the oral cavity, to the surface of the teeth or any combination thereof. Examples of oral conditions such oral care actives address include, but are not limited to, appearance and structural changes to teeth, whitening, stain bleaching, stain removal, plaque removal, tartar removal, cavity prevention and treatment, inflamed and/or bleeding gums, mucosal wounds, lesions, ulcers, aphthous ulcers, cold sores and tooth abscesses, oral malodor, dental erosion, gingivitis, and/or periodontal disease. Oral conditions are further described in WO 02/02096A2.

The photocatalyzable consumer product composition may include one or more oral care additives. The oral care active can be any material that is generally recognized as safe for use in the oral cavity that provides changes to the overall health of the oral cavity, and specifically the condition of the oral surfaces the oral care active contacts. The photocatalyzable consumer product composition can comprise one or multiple oral care additives.

It is also contemplated that a single oral care product can comprise multiple photocatalyzable consumer product compositions, each of which comprises one or more oral care additives Some oral care additives that are suitable for use in the photocatalyzable consumer product composition are discussed more fully below.

The photocatalyzable consumer product composition may include one or more gelling agents, which may also act as an adhesive agent to adhere the photocatalyzable consumer product composition to the plurality of teeth. The concentration of the gelling agent may be greater than about 2, 4, 6, 8, 10, 15, 20, 30, 40, 50, 60 or less than about 80, 70, 60, 50, 40, 30, or 20 percent by weight of the photocatalyzable consumer product composition.

Suitable gelling agents and/or adhesion agents useful in the present invention are described in U.S. Pat. Nos. 6,649,147; 6,780,401; 2004/0102554; 2005/0089819; 2003/0152528; 6,419,906; and 2005/0100515. Some of the gelling agents or adhesion agents may include silicone, polyethylene oxide, polyvinyl alcohol, poly alkyl vinyl ether-maleic acid copolymer (PVM/MA copolymer) such as, Gantrez AN 119, AN 139, and S-97, polyvinyl alcohol, polyacrylic acid, Poloxamer 407 (Pluronic), polyvinyl pyrrolidone-vinyl acetate copolymer (PVP/VA copolymer), such as Luviskol VA, and Plasdone S PVP/VA, polyvinyl pyrrolidone (PVP, e.g., K-15 to K-120), Polyquaterium-11 (Gafquat 755N), Polyquaterium-39 (Merquat plus 3330), carbomer or carboxypolymethylene (Carbopol), hydroxy propyl methyl cellulose, hydroxy ethyl cellulose, hydroxy propyl cellulose, carboxymethyl cellulose, gelatin and alginate salt such as sodium alginate, natural gums such as gum karaya, xanthan gum, Guar gum, gum arabic, gum tragacanth, and mixtures thereof.

A humectant or plasticizer may be included in the photocatalyzable consumer product composition, including glycerin, sorbitol, polyethylene glycol, propylene glycol, and other edible polyhydric alcohols. The humectants may be present between about 10% to about 95%, or between about 50% and about 80%, by weight of the photocatalyzable consumer product composition. A photocatalyzable consumer product composition can also include flavoring agents, sweetening agents, opacifiers, and coloring agents.

The photocatalyzable consumer product composition of the present invention may comprise a non-photocatalyzable tooth whitening agent in addition to any benefit active precursor. The photocatalyzable consumer product composition comprising a non-photocatalyzable tooth whitening active can be provided in a variety of liquid forms, such as a viscous liquid, a paste, a gel, or a solution. Preferably, the oral care composition comprising a non-photocatalyzable tooth whitening active is in the form of a gel. The oral care composition comprising a non-photocatalyzable tooth whitening active may have a viscosity between about 200 and about 5,000,000 cps at low shear rates (less than one 1/seconds). In one embodiment, the viscosity may be between about 100,000 and about 1,500,000 cps and in another embodiment between about 400,000 and about 1,000,000 cps.

A polymeric mesh or scrim may be incorporated in the photocatalyzable consumer product composition comprising a non-photocatalyzable tooth whitening active. The non-photocatalyzable tooth whitening actives that may be suitable for use in the oral care composition include peroxides, perborates, percarbonates, peroxyacids, and combinations thereof. Suitable peroxide compounds include hydrogen peroxide, calcium peroxide, carbamide peroxide, and mixtures thereof. The photocatalyzable consumer product composition may contain a non-photocatalyzable tooth whitening active at greater than about 2, 4, 6, 8, 10, 15, 20% and/or less than about 40, 25, 20, 18, 16, 14, 12, or 10% by weight of the photocatalyzable consumer product composition.

It is desirable to use a photocatalyzable consumer product composition that is highly tolerable to the surfaces of the oral cavity and minimizes the generation of tooth sensitivity. An example of a photocatalyzable consumer product composition that is tolerable to the soft tissue of the oral cavity and minimizes the generation of tooth sensitivity, includes one with a peroxide concentration of less than about 7.5% peroxide by weight of the total tooth whitening composition and a peroxide density of less than about 1.3 mg/cm². In one embodiment the photocatalyzable consumer product composition has a peroxide concentration of greater than about 0.01% and less than about 7.5% peroxide by weight of the photocatalyzable consumer product composition and a peroxide density of less than about 1.3 mg/cm². In another embodiment the photocatalyzable consumer product composition has a peroxide concentration of greater than about 6.5% and less than about 7.5% peroxide by weight of the photocatalyzable consumer product composition and a peroxide density of less than about 1.3 mg/cm². In another embodiment the photocatalyzable consumer product composition has a peroxide concentration of greater than about 6.0% and less than about 6.5% peroxide by weight of the photocatalyzable consumer product composition and a peroxide density of less than about 1.3 mg/cm², and in yet another embodiment the photcatalyzable consumer product composition has a peroxide concentration of less than about 6.0% peroxide by weight of the photocatalyzable consumer product composition and a peroxide density of less than about 1.3 mg/cm²

Peroxide density is the ratio of the amount of peroxide active (mg) or peroxide dose to the surface area (cm²) of the thin layer that is applied to the tooth surfaces and adjacent soft tissue of the oral cavity. Peroxide density and the calculation thereof is discussed in U.S. Pat. No. 6,949,240. If the photocatalyzable consumer product composition contains 6.5% hydrogen peroxide and 0.2 g of the photocatalyzable consumer product composition is applied, then the hydrogen peroxide dose is 13 mg. The corresponding peroxide density is 1.3 mg/cm².

The photocatalyzable consumer product composition of the present invention may comprise a non-photocatalyzable anti-tartar agent. Anti-tartar actives known for use in dental care products includes phosphates. Phosphates include pyrophosphates, polyphosphates, polyphosphates and mixtures thereof. Pyrophosphates are among the best known for use in dental care products. Pyrophosphate ions are delivered to the teeth derive from pyrophosphate salts. The pyrophosphate salts useful in the present compositions include the dialkali metal pyrophosphate salts, tetra-alkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. While any of the above mentioned pyrophosphate salts may be used, tetrasodium pyrophosphate salt is preferred. In one embodiment the photocatalyzable consumer product composition comprises from about 0.5% to about 5% of a pyrophosphate by weight of the photocatalyzable consumer product composition. In another embodiment the photocatalyzable consumer product composition comprises from about 0.5% to about 3% of a pyrophosphate by weight of the photocatalyzable consumer product composition.

The pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated into Kirk & Othmer. Additional anticalculus actives include pyrophosphates or polyphosphates disclosed in U.S. Pat. No. 4,590,066 issued to Parran & Sakkab on May 20, 1986; polyacrylates and other polycarboxylates such as those disclosed in U.S. Pat. No. 3,429,963 issued to Shedlovsky on Feb. 25, 1969 and U.S. Pat. No. 4,304,766 issued to Chang on Dec. 8, 1981; and U.S. Pat. No. 4,661,341 issued to Benedict & Sunberg on Apr. 28, 1987; polyepoxysuccinates such as those disclosed in U.S. Pat. No. 4,846,650 issued to Benedict, Bush & Sunberg on Jul. 11, 1989; ethylenediaminetetraacetic acid as disclosed in British Patent No. 490,384 dated Feb. 15, 1937; nitrilotriacetic acid and related compounds as disclosed in U.S. Pat. No. 3,678,154 issued to Widder & Briner on Jul. 18, 1972; polyphosphonates as disclosed in U.S. Pat. No. 3,737,533 issued to Francis on Jun. 5, 1973, U.S. Pat. No. 3,988,443 issued to Ploger, Schmidt-Dunker & Gloxhuber on Oct. 26, 1976 and U.S. Pat. No. 4,877,603 issued to Degenhardt & Kozikowski on Oct. 31, 1989; all of these patents are incorporated herein by reference. Anticalculus phosphates include potassium and sodium pyrophosphates; sodium tripolyphosphate; diphosphonates, such as ethane-1-hydroxy-1,1-diphosphonate, 1-azacycloheptane-1,1-diphosphonate, and linear alkyl diphosphonates; linear carboxylic acids; and sodium zinc citrate.

Actives that may be used in place of or in combination with the pyrophosphate salt include such known materials as synthetic anionic polymers including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Other anti-tartar actives include sodium hexametaphosphate.

The photocatalyzable consumer product composition of the present invention may also comprise a non-photocatalyzable anti-caries agent. Fluoride ion sources are well known for use in oral care compositions as anticaries Actives. Fluoride ions are contained in a number of oral care compositions for this purpose, particularly toothpastes. Patents disclosing such toothpastes include U.S. Pat. No. 3,538,230, Nov. 3, 1970 to Pader et al; U.S. Pat. No. 3,689,637, Sep. 5, 1972 to Pader; U.S. Pat. No. 3,711,604, Jan. 16, 1973 to Colodney et al; U.S. Pat. No. 3,911,104, Oct. 7, 1975 to Harrison; U.S. Pat. No. 3,935,306, Jan. 27, 1976 to Roberts et al; and U.S. Pat. No. 4,040,858, Aug. 9, 1977 to Wason.

Application of fluoride ions to dental enamel serves to protect teeth against decay. A wide variety of fluoride ion-yielding materials can be employed as sources of soluble fluoride in the instant photocatalyzable consumer product compositions. Examples of suitable fluoride ion-yielding materials are found in Briner et al; U.S. Pat. No. 3,535,421; issued Oct. 20, 1970 and Widder et al; U.S. Pat. No. 3,678,154; issued Jul. 18, 1972, both patents being incorporated herein by reference. Preferred fluoride ion sources for use herein include stannous fluoride, monofluorophosphate, sodium fluoride, potassium fluoride and ammonium fluoride. Sodium fluoride is particularly preferred. Preferably the instant photocatalyzable consumer product compositions provide from about 50 ppm to 10,000 ppm, more preferably from about 100 to 3000 ppm, of fluoride ions in the aqueous solutions that contact dental surfaces when used with the strip of material used in the mouth. Other anti-caries actives include xylitol.

The photocatalyzable consumer product composition of the present invention may comprise a non-photocatalyzable antimicrobial agent. Non-photocatalyzable antimicrobial agents may include, but are not limited to, 5-chloro-2-(2,4-dichlorophenoxy)-phenol, commonly referred to as triclosan, and described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; phthalic acid and its salts including, but not limited to those disclosed in U.S. Pat. No. 4,994,262, Feb. 19, 1991, substituted monoperthalic acid and its salts and esters as disclosed in U.S. Pat. No. 4,990,329, Feb. 5, 1991, 5,110,583, May 5, 1992 and U.S. Pat. No. 4,716,035, Dec. 29, 1987, all to Sampathkumar; preferably magnesium monoperoxy phthalate, chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicyanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion actives; antibiotics such as augmentin, amoxicillin, tetracycline, doxycycline, minocycline, and metronidazole; and analogs and salts of the above; essential oils including thymol, geraniol, carvacrol, citral, hinokitiol, eucalyptol, catechol (particularly 4-allyl catechol), metals or metal ions (e.g., silver, copper, zinc, etc) and mixtures thereof; methyl salicylate; chlorite and metal salts of chlorite and mixtures of all of the above.

The photocatalyzable consumer product composition of the present invention may comprise a non-photocatalyzable anti-inflammatory or non-photocatalyzable anti-sensitivity agent. Anti-inflammatory agents may include, but are not limited to, non-steroidal anti-inflammatory actives or NSAIDs such as ketorolac, flurbiprofen, ibuprofen, naproxen, indomethacin, aspirin, ketoprofen, piroxicam and meclofenamic acid. Use of NSAIDs such as Ketorolac are claimed in U.S. Pat. No. 5,626,838, issued May 6, 1997, herein incorporated by reference. Disclosed therein are methods of preventing and, or treating primary and reoccurring squamous cell carcinoma of the oral cavity or oropharynx by topical administration to the oral cavity or oropharynx an effective amount of an NSAID.

Anti-sensitivity agents can include potassium nitrate, clove oil (Eugenol) and other herbal or flavor actives/agents.

Nutrients may improve the condition of the oral cavity and can be included in the photocatalyzable consumer product compositions. The photocatalyzable consumer product composition of the present invention may comprise a non-photocatalyzable nutrient adjunt include minerals, vitamins, oral nutritional supplements, enteral nutritional supplements, and mixtures thereof.

Minerals that can be included with the photocatalyzable consumer product compositions of the present invention include calcium, phosphorus, fluoride, zinc, manganese, potassium and mixtures thereof. These minerals are disclosed in Drug Facts and Comparisons (loose leaf drug information service), Wolters Kluer Company, St. Louis, Mo., .COPYRIGHT. 1997, pp 10-17; incorporated herein by reference.

Vitamins can be included with minerals or used separately. Vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Such vitamins are disclosed in Drug Facts and Comparisons (loose leaf drug information service), Wolters Kluer Company, St. Louis, Mo., .COPYRIGHT. 1997, pp. 3-10; incorporated herein by reference.

Oral nutritional supplements include amino acids, lipotropics, fish oil, and mixtures thereof, as disclosed in Drug Facts and Comparisons (loose leaf drug information service), Wolters Kluer Company, St. Louis, Mo., .COPYRIGHT. 1997, pp. 54-54e; incorporated herein by reference. Amino acids include, but, are not limited to L-Tryptophan, L-Lysine, Methionine, Threonine, Levocamitine or L-carnitine and mixtures thereof. Lipotropics include, but, are not limited to choline, inositol, betaine, linoleic acid, linolenic acid, and mixtures thereof. Fish oil contains large amounts of Omega-3 (N-3) Polyunsaturated fatty acids, eicosapentaenoic acid and docosahexaenoic acid.

Entenal nutritional supplements include, but, are not limited to protein products, glucose polymers, corn oil, safflower oil, medium chain triglycerides as disclosed in Drug Facts and Comparisons (loose leaf drug information service), Wolters Kluer Company, St. Louis, Mo., .COPYRIGHT. 1997, pp. 55-57; incorporated herein by reference.

The photocatalyzable consumer product composition of the present invention may comprise non-photocatalyzable enzymes. An individual or combination of several compatible enzymes can be included in the photocatalyzable consumer product composition. Enzymes are biological catalysts of chemical reactions in living systems. Enzymes combine with the substrates on which they act forming an intermediate enzyme-substrate complex. This complex is then converted to a reaction product and a liberated enzyme which continues its specific enzymatic function.

Enzymes provide several benefits when used for cleansing of the oral cavity. Proteases break down salivary proteins which are absorbed onto the tooth surface and form the pellicle; the first layer of resulting plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking-up the carbohydrate-protein complex that binds calcium, preventing mineralization.

Enzymes useful in the present invention include any of the commercially available proteases, glucanohydrolases, endoglycosidases, amylases, mutanases, lipases and mucinases or compatible mixtures thereof. Preferred are the proteases, dextranases, endoglycosidases and mutanases, most preferred being papain, endoglycosidase or a mixture of dextranase and mutanase. Additional enzymes suitable for use in the present invention are disclosed in U.S. Pat. No. 5,000,939 to Dring et al., Mar. 19, 1991; U.S. Pat. No. 4,992,420 to Neeser, Feb. 12, 1991; U.S. Pat. No. 4,355,022 to Rabussay, Oct. 19, 1982; U.S. Pat. No. 4,154,815 to Pader, May 15, 1979; U.S. Pat. No. 4,058,595 to Colodney, Nov. 15, 1977; U.S. Pat. No. 3,991,177 to Virda et al., Nov. 9, 1976 and U.S. Pat. No. 3,696,191 to Weeks, Oct. 3, 1972; all incorporated herein by reference.

The photocatalyzable consumer product composition of the present invention may comprise commonly known mouth and throat products. Such products are disclosed in Drug Facts and Comparisons (loose leaf drug information service), Wolters Kluer Company, St. Louis, Mo., .COPYRIGHT. 1997, pp. 520b-527; incorporated herein by reference. These products include, but, are not limited to anti-fungal, antibiotic and analgesic actives.

### Hard Surface and Dish Additives

It would be appreciated by one of ordinary skill in the art that many of the adjunct additives useful in any of the preceeding product types and hard surface and dish products may generally fall into similar classes, though the specific preferred materials may differ.

### Surfactant

Surfactants may be desired herein as they contribute to the consumer product performance of the photocatalyzable compositions of the present invention. Suitable surfactants are selected from the group consisting of a nonionic surfactant or a mixture thereof; an anionic surfactant or a mixture thereof; an amphoteric surfactant or a mixture thereof; a zwitterionic surfactant or a mixture thereof; a cationic surfactant or a mixture thereof; and mixtures thereof.

In a preferred embodiment wherein the composition is a hard surface consumer product composition, the composition comprises from about 1% to about 60%, preferably from about 5% to about 30%, and more preferably from about 10% to about 25% by weight of the total composition of a surfactant.

In a preferred embodiment wherein the composition is a dishwashing detergent composition, the composition may comprise from about 5% to about 80%, preferably from about 10% to about 60%, more preferably from about 12% to about 45% by weight of the total composition of a surfactant. In preferred embodiments, the surfactant herein has an average branching of the alkyl chain(s) of more than about 10%, preferably more than about 20%, more preferably more than about 30%, and even more preferably more than about 40% by weight of the total surfactant.

### Nonionic Surfactant

In one preferred embodiment, the photocatalyzable consumer product composition comprises a nonionic surfactant. Suitable nonionic surfactants may be alkoxylated alcohol nonionic surfactants. A great variety of such alkoxylated alcohols, especially ethoxylated and/or propoxylated alcohols, are commercially available. Preferred alkoxylated alcohols for use herein are nonionic surfactants according to the formula R¹O(E)ₑ(P)ₚH where R¹ is a hydrocarbon chain of from about 2 to about 24 carbon atoms, E is ethylene oxide, P is propylene oxide, and e and p which represent the average degree of, respectively ethoxylation and propoxylation, are of from about 0 to about 24 (with the sum of e+p being at least 1). Preferably, the hydrophobic moiety of the nonionic compound can be a primary or secondary, straight or branched alcohol having from about 8 to about 24 carbon atoms.

In some embodiments, preferred nonionic surfactants are the condensation products of ethylene oxide and/or propylene oxide with an alcohol having a straight or branched alkyl chain, having from about 6 to about 22 carbon atoms, preferably from about 9 to about 15 carbon atoms, wherein the degree of alkoxylation (ethoxylation and/or propoxylation) is from about 1 to about 25, preferably from about 2 to about 18, and more preferably from about 5 to about 12 moles of alkylene oxide per mole of alcohol. Particularly preferred are such surfactants containing from about 5 to about 12 moles of ethylene oxide per mole of alcohol. Such suitable nonionic surfactants are commercially available from Shell, for instance, under the trade name Neodol® or from BASF under the trade name Lutensol®.

Preferably, the nonionic surfactant is comprised in a typical amount of from about 2% to about 40%, preferably from about 3% to about 30% by weight of the photocatalyzable consumer product composition, and preferably from about 3 to about 20% by weight of the total composition.

Also suitable are alkylpolyglycosides having the formula R³O(CₙH₂ₙO)ₜ(glycosyl)_{z} (formula (III)), wherein R³ of formula (III) is selected from the group consisting of an alkyl or a mixture thereof; an alkyl-phenyl or a mixture thereof; a hydroxyalkyl or a mixture thereof; a hydroxyalkylphenyl or a mixture thereof; and mixtures thereof, in which the alkyl group contains from about 10 to about 18, preferably from about 12 to about 14 carbon atoms; n of formula (III) is about 2 or about 3, preferably about 2; t of formula (III) is from about 0 to about 10, preferably about 0; and z of formula (III) is from about 1.3 to about 10, preferably from about 1.3 to about 3, most preferably from about 1.3 to about 2.7. The glycosyl is preferably derived from glucose.

Also suitable are alkyl glycerol ether and sorbitan ester. Also suitable is fatty acid amide surfactant having the formula (IV):

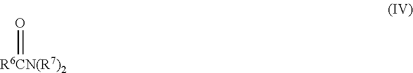

wherein R⁶ of formula (IV) is an alkyl group containing from about 7 to about 21, preferably from about 9 to about 17, carbon atoms, and each R⁷ of formula (IV) is selected from the group consisting of hydrogen; a C₁-C₄ alkyl or a mixture thereof; a C₁-C₄ hydroxyalkyl or a mixture thereof; and a —(C₂H₄O)_{y}H or a mixture thereof, where y of formula (IV) varies from about 1 to about 3. Preferred amide can be a C₈-C₂₀ ammonia amide, a monoethanolamide, a diethanolamide, and an isopropanolamide.

Other preferred nonionic surfactants for use in the photocatalyzable consumer product composition may be the mixture of nonyl (C₉), decyl (C₁₀) undecyl (C₁₁) alcohols modified with, on average, about 5 ethylene oxide (EO) units such as the commercially available Neodol 91-5® or the Neodol 91-8® that is modified with on average about 8 EO units. Also suitable are the longer alkyl chains ethoxylated nonionics such as C₁₂ or C₁₃ modified with 5 EO (Neodol 23-5®). Neodol® is a Shell tradename. Also suitable is the C₁₂ or C₁₄ alkyl chain with 7 EO, commercially available under the trade name Novel 1412-7® (Sasol) or the Lutensol A 7 N® (BASF).

Preferred branched nonionic surfactants are the Guerbet C₁₀ alcohol ethoxylates with 5 EO such as Ethylan 1005, Lutensol XP 50® and the Guerbet C₁₀ alcohol alkoxylated nonionics (modified with EO and PO (propylene oxide)) such as the commercially available Lutensol XL® series (X150, XL70, etc). Other branching also includes oxo branched nonionic surfactants such as the Lutensol ON 50® (5 EO) and Lutensol ON70® (7 EO). Other suitable branched nonionics are the ones derived from the isotridecyl alcohol and modified with ethylene oxide such as the Lutensol TO7® (7EO) from BASF and the Marlipal O 13/70® (7 EO) from Sasol. Also suitable are the ethoxylated fatty alcohols originating from the Fisher & Tropsch reaction comprising up to about 50% branching (about 40% methyl (mono or bi) about 10% cyclohexyl) such as those produced from the Safol® alcohols from Sasol; ethoxylated fatty alcohols originating from the oxo reaction wherein at least 50 wt % of the alcohol is C₂ isomer (methyl to pentyl) such as those produced from the Isalchem® alcohols or Lial® alcohols from Sasol; the ethoxylated fatty alcohols originating from the modified oxo reaction wherein at least about 15% by weight of the alcohol is C₂ isomer (methyl to pentyl) such as those produced from the Neodol® alcohols from Shell.

In one preferred embodiment, the weight ratio of total surfactant to nonionic surfactant is from about 2 to about 10, preferably from about 2 to about 7.5, more preferably from about 2 to about 6.

### Anionic Surfactant

Suitable anionic surfactants for use in the photocatalyzable consumer product composition can be a sulfate, a sulfosuccinate, a sulfoacetate, and/or a sulphonate; preferably an alkyl sulfate and/or an alkyl ethoxy sulfate; more preferably a combination of an alkyl sulfate and/or an alkyl ethoxy sulfate with a combined ethoxylation degree less than about 5, preferably less than about 3, more preferably less than about 2.

Sulphate or sulphonate surfactant is typically present at a level of at least about 5%, preferably from about 5% to about 40%, and more preferably from about 15% to about 30%, and even more preferably at about 15% to about 25% by weight of the photocatalyzable consumer product composition.

Suitable sulphate or sulphonate surfactants for use in the photocatalyzable consumer product composition include water-soluble salts or acids of C₈-C₁₄ alkyl or hydroxyalkyl, sulphate or sulphonates. Suitable counterions include hydrogen, alkali metal cation or ammonium or substituted ammonium, but preferably sodium. Where the hydrocarbyl chain is branched, it preferably comprises a C₁₋₄ alkyl branching unit. The average percentage branching of the sulphate or sulphonate surfactant is preferably greater than about 30%, more preferably from about 35% to about 80%, and most preferably from about 40% to about 60% of the total hydrocarbyl chain. One particularly suitable linear alkyl sulphonate includes C₈ sulphonate like Witconate NAS 8® commercially available from Witco.

The sulphate or sulphonate surfactants may be selected from a C₁-C₁₈ alkyl benzene sulphonate (LAS), a C₈-C₂₀ primary, a branched-chain and random alkyl sulphate (AS); a C₁₀-C₁₈ secondary (2,3) alkyl sulphate; a C₁₀-C₁₈ alkyl alkoxy sulphate (AEₓS) wherein preferably x is from 1-30; a C₁₀-C₁₈ alkyl alkoxy carboxylate preferably comprising about 1-5 ethoxy units; a mid-chain branched alkyl sulphate as discussed in U.S. Pat. No. 6,020,303 and U.S. Pat. No. 6,060,443; a mid-chain branched alkyl alkoxy sulphate as discussed in U.S. Pat. No. 6,008,181 and U.S. Pat. No. 6,020,303; a modified alkylbenzene sulphonate (MLAS) as discussed in WO 99/05243, WO 99/05242, WO 99/05244, WO 99/05082, WO 99/05084, WO 99/05241, WO 99/07656, WO 00/23549, and WO 00/23548; a methyl ester sulphonate (MES); and an alpha-olefin sulphonate (AOS).

The paraffin sulphonate may be monosulphonate or disulphonate and usually are mixtures thereof, obtained by sulphonating a paraffin of about 10 to about 20 carbon atoms. Preferred sulphonates are those of C₁₂₋₁₈ carbon atoms chains and more preferably they are C₁₄₋₁₇ chains. Paraffin sulphonates that have the sulphonate group(s) distributed along the paraffin chain are described in U.S. Pat. No. 2,503,280; U.S. Pat. No. 2,507,088; U.S. Pat. No. 3,260,744; and U.S. Pat. No. 3,372,188.

Also suitable are the alkyl glyceryl sulphonate surfactant and/or alkyl glyceryl sulphate surfactant described in the Procter & Gamble patent application WO06/014740: A mixture of oligomeric alkyl glyceryl sulphonate and/or sulfate surfactant selected from a dimmer or a mixture thereof; a trimer or a mixture thereof; a tetramer or a mixture thereof; a pentamer or a mixture thereof; a hexamer or a mixture thereof; a heptamer or a mixture thereof; and mixtures thereof; wherein the alkyl glyceryl sulphonate and/or sulfate surfactant mixture comprises from about 0% to about 60% by weight of the monomers.

Other suitable anionic surfactants are alkyl, preferably dialkyl sulfosuccinate and/or sulfoacetate. The dialkyl sulfosuccinate may be a C₆₋₁₅ linear or branched dialkyl sulfosuccinate. The alkyl moiety may be symmetrical (i.e., the same alkyl moieties) or asymmetrical (i.e., different alkyl moieties). Preferably, the alkyl moiety is symmetrical.

Most common branched anionic alkyl ether sulphates are obtained via sulfation of a mixture of the branched alcohols and the branched alcohol ethoxylates. Also suitable are the sulfated fatty alcohols originating from the Fischer & Tropsh reaction comprising up to about 50% branching (about 40% methyl (mono or bi) about 10% cyclohexyl) such as those produced from the safol alcohols from Sasol; sulfated fatty alcohols originating from the oxo reaction wherein at least about 50% by weight of the alcohol is C₂ isomer (methyl to pentyl) such as those produced from the Isalchem® alcohols or Lial® alcohols from Sasol; the sulfated fatty alcohols originating from the modified oxo reaction wherein at least about 15% by weight of the alcohol is C₂ isomer (methyl to pentyl) such as those produced from the Neodol® alcohols from Shell.

### Zwitterionic Surfactant and Amphoteric Surfactant

The zwitterionic and amphoteric surfactants for use in the photocatalyzable consumer product composition can be comprised at a level of from about 0.01% to about 20%, preferably from about 0.2% to about 15%, more preferably from about 0.5% to about 10% by weight of photocatalyzable consumer product composition.

Suitable zwitterionic surfactant in the preferred embodiment wherein contains both basic and acidic groups which form an inner salt giving both cationic and anionic hydrophilic groups on the same molecule at a relatively wide range of pH's. The typical cationic group is a quaternary ammonium group, although other positively charged groups like phosphonium, imidazolium and sulfonium groups can be used. The typical anionic hydrophilic groups are carboxylate and sulphonate, although other groups like sulfate, phosphonate, and the like can be used.

The photocatalyzable consumer product composition may preferably further comprise an amine oxide and/or a betaine. Most preferred amine oxides are coconut dimethyl amine oxide or coconut amido propyl dimethyl amine oxide. Amine oxide may have a linear or mid-branched alkyl moiety. Typical linear amine oxides include water-soluble amine oxide containing one R⁴C₈₋₁₈ alkyl moiety and 2 R⁵ and R⁸ moieties selected from the group consisting of a C₁₋₃ alkyl group and a mixtures thereof; and a C₁₋₃ hydroxyalkyl group and a mixture thereof. Preferably amine oxide is characterized by the formula R⁴—N(R⁵)(R⁸)→O wherein R⁴ is a C₈₋₁₈ alkyl and R⁵ and R⁸ are selected from the group consisting of a methyl; an ethyl; a propyl; an isopropyl; a 2-hydroxethyl; a 2-hydroxypropyl; and a 3-hydroxypropyl. The linear amine oxide surfactant, in particular, may include a linear C₁₀-C₁₈ alkyl dimethyl amine oxide and a linear C₈-C₁₂ alkoxy ethyl dihydroxy ethyl amine oxide. Preferred amine oxides include linear C₁₀, linear C₁₀-C₁₂, and linear C₁₂-C₁₄ alkyl dimethyl amine oxides.

As used herein “mid-branched” means that the amine oxide has one alkyl moiety having n₁ carbon atoms with one alkyl branch on the alkyl moiety having n₂ carbon atoms. The alkyl branch is located on the a carbon from the nitrogen on the alkyl moiety. This type of branching for the amine oxide is also known in the art as an internal amine oxide. The total sum of n₁ and n₂ is from about 10 to about 24 carbon atoms, preferably from about 12 to about 20, and more preferably from about 10 to about 16. The number of carbon atoms for the one alkyl moiety (n₁) should be approximately the same number of carbon atoms as the one alkyl branch (n₂) such that the one alkyl moiety and the one alkyl branch are symmetric. As used herein, “symmetric” means that |n₁−n₂| is less than or equal to about 5, preferably about 4, most preferably from about 0 to about 4 carbon atoms in at least about 50 wt %, more preferably at least about 75 wt % to about 100 wt % of the mid-branched amine oxide for use herein.

The amine oxide further comprises two moieties, independently selected from a C₁₋₃ alkyl; a C₁₋₃ hydroxyalkyl group; or a polyethylene oxide group containing an average of from about 1 to about 3 ethylene oxide groups. Preferably the two moieties are selected from a C₁₋₃ alkyl, more preferably both are selected as a C₁ alkyl.

Other suitable surfactants include a betaine such an alkyl betaine, an alkylamidobetaine, an amidazoliniumbetaine, a sulfobetaine (INCI Sultaines), as well as a phosphobetaine, and preferably meets formula I:

R^{1′}—[CO—X(CH₂)ⱼ]_{g}—N⁺(R^{2′})(R^{3′})—(CH₂)_{f}[CH(OH)—CH₂]ₕ—Y— (I)

wherein
R^{1′} is a saturated or unsaturated C₆₋₂₂ alkyl residue, preferably a C₈₋₁₈ alkyl residue, in particular a saturated C₁₀₋₁₆ alkyl residue, for example a saturated C₁₂₋₁₄ alkyl residue;

X is NH, NR^{4′} with C₁₋₄ alkyl residue R^{4′}, O or S,
j is a number from about 1 to about 10, preferably from about 2 to about 5, in particular about 3,
g is about 0 or about 1, preferably about 1,
R^{2′}, R^{3′} are independently a C₁₋₄ alkyl residue, potentially hydroxy substituted by such as a hydroxyethyl, preferably by a methyl.
f is a number from about 1 to about 4, in particular about 1, 2 or 3,
h is about 0 or 1, and
Y is selected from COO, SO₃, OPO(OR^{5′})O or P(O)(OR^{5′})O, whereby R^{5′} is a hydrogen atom H or a C₁₋₄ alkyl residue.

Preferred betaines are the alkyl betaine of the formula (Iₐ), the alkyl amido betaine of the formula (I_{b}), the sulfo betaine of the formula (Iₐ), and the Amido sulfobetaine of the formula (I_{d});

R^{1′}—N⁺(CH₃)₂—CH₂COO⁻ (Iₐ)

R^{1′}—CO—NH(CH₂)₃—N⁺(CH₃)₂—CH₂COO⁻ (I_{b})

R^{1′}—N⁺(CH₃)₂—CH₂CH(OH)CH₂SO₃— (I_{c})

R^{1′}—CO—NH—(CH₂)₃—N⁺(CH₃)₂—CH₂CH(OH)CH₂SO₃⁻ (I_{d})

in which R^{1′} has the same meaning as in formula I. Particularly preferred betaines are the carbobetaine, wherein Y⁻ is [COO⁻], in particular the carbobetaine of formula (Iₐ) and (I_{b}), more preferred are the alkylamidobetaine of the formula (I_{b}).

Examples of suitable betaines and sulfobetaines are the following (designated in accordance with INCI): almondamidopropyl of betaine, apricotamidopropyl betaine, avocadamidopropyl of betaine, babassuamidopropyl of betaine, behenamidopropyl betaine, behenyl of betaine, betaine, canolamidopropyl betaine, capryl/capramidopropyl betaine, carnitine, cetyl of betaine, cocamidoethyl of betaine, cocamidopropyl betaine, cocamidopropyl hydroxysultaine, coco betaine, coco hydroxysultaine, coco/oleamidopropyl betaine, coco sultaine, decyl of betaine, dihydroxyethyl oleyl glycinate, dihydroxyethyl soy glycinate, dihydroxyethyl stearyl glycinate, dihydroxyethyl tallow glycinate, dimethicone propyl of PG-betaine, drucamidopropyl hydroxysultaine, hydrogenated tallow of betaine, isostearamidopropyl betaine, lauramidopropyl betaine, lauryl of betaine, lauryl hydroxysultaine, lauryl sultaine, milk amidopropyl betaine, milkamidopropyl of betaine, myristamidopropyl betaine, myristyl of betaine, oleamidopropyl betaine, oleamidopropyl hydroxysultaine, oleyl of betaine, olivamidopropyl of betaine, palmamidopropyl betaine, palmitamidopropyl betaine, palmitoyl carnitine, palm kernel amidopropyl betaine, polytetrafluoroethylene acetoxypropyl of betaine, ricinoleamidopropyl betaine, sesamidopropyl betaine, soyamidopropyl betaine, stearamidopropyl betaine, stearyl of betaine, tallowamidopropyl betaine, tallowamidopropyl hydroxysultaine, tallow of betaine, tallow dihydroxyethyl of betaine, undecylenamidopropyl betaine and wheat germ amidopropyl betaine. Preferred betaine is for example cocoamidopropyl betaine.

For example coconut dimethyl betaine is commercially available from Seppic under the trade name of Amonyl 265®. Lauryl betaine is commercially available from Albright & Wilson under the trade name Empigen BB/L®. A further example of betaine is lauryl-imino-dipropionate commercially available from Rhodia under the trade name Mirataine H2C-HA®.

One particularly preferred zwitterionic surfactants for use in the preferred embodiment wherein the composition is a hard surface consumer product composition is the sulfobetaine surfactant, because it delivers optimum soap scum consumer product benefits.

Examples of particularly suitable sulfobetaine surfactants include tallow bis(hydroxyethyl) sulphobetaine and cocoamido propyl hydroxy sulphobetaine which are commercially available from Rhodia and Witco, under the trade name of Mirataine CBS® and Rewoteric AM CAS 15® respectively.

### Cationic Surfactant

In one preferred embodiment, the photocatalyzable consumer product composition can comprise a cationic surfactant present in an effective amount, more preferably from about 0.1% to about 20%, by weight of photocatalyzable consumer product composition. Suitable cationic surfactant is quaternary ammonium surfactant. Suitable quaternary ammonium surfactant is selected from the group consisting of a mono C₆-C₁₆, preferably a C₆-C₁₀ N-alkyl or an alkenyl ammonium surfactant or a mixture thereof, wherein the remaining N positions are substituted by a methyl, a hydroxyethyl or a hydroxypropyl group. Another preferred cationic surfactant is a C₆-C₁₈ alkyl or alkenyl ester of a quaternary ammonium alcohol, such as quaternary chlorine ester. More preferably, the cationic surfactant has formula (V):

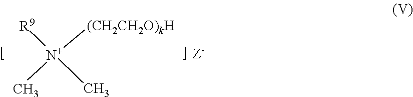

wherein R⁹ of formula (V) is a C₈-C₁₈ hydrocarbyl or a mixture thereof, preferably, a C₈₋₁₄ alkyl, more preferably, a C₈, C₁₀ or C₁₂ alkyl; and Z of formula (V) is an anion, preferably, a chloride or a bromide.

### Optional Ingredients

The photocatalyzable consumer product composition according to the present invention may comprise a variety of optional ingredients depending on the technical benefit aimed for and the surfaces treated.

Suitable optional ingredients for use herein include an alkaline material or a mixture thereof; an inorganic or organic acid and salt thereof or a mixture thereof; a buffering agent or a mixture thereof; a surface modifying polymer or a mixture thereof; a consumer product polymer or a mixture thereof; a peroxygen bleach or a mixture thereof; a radical scavenger or a mixture thereof; a chelating agent or a mixture thereof; a perfume or a mixture thereof; a dye or a mixture thereof; a hydrotrope or a mixture thereof; a polymeric suds stabilizer or a mixture thereof; a diamine or a mixture thereof; and mixtures thereof.

### Solvent

Solvents are generally used to ensure preferred product quality for dissolution, thickness and aesthetics and to ensure better processing. The photocatalyzable consumer product composition of the present invention may further comprise a solvent or a mixture thereof, as an optional ingredient. Typically, in the preferred embodiment wherein the composition is a hard surface consumer product composition, the composition may comprise from about 0.1% to about 10%, preferably from about 0.5% to about 5%, and more preferably from about 1% to about 3% by weight of the total composition of a solvent or a mixture thereof. In the preferred embodiment wherein the composition is a hand dishwashing detergent composition, the composition contains from about 0.01% to about 20%, preferably from about 0.5% to about 20%, more preferably from about 1% to about 10% by weight of a solvent.

Suitable solvents herein include C₁-C₅ alcohols according to the formula R¹⁰—OH wherein R¹⁰ is a saturated alkyl group of from about 1 to about 5 carbon atoms, preferably from about 2 to about 4. Suitable alcohols are ethanol, propanol, isopropanol or mixtures thereof. Other suitable alcohols are alkoxylated C₁₋₈ alcohols according to the formula R¹¹-(A_{q})-OH wherein R¹¹ is a alkyl group of from about 1 to about 8 carbon atoms, preferably from about 3 to about 6, and wherein A is an alkoxy group, preferably propoxy and/or ethoxy, and q is an integer of from 1 to 5, preferably from 1 to 2. Suitable alcohols are butoxy propoxy propanol (n-BPP), butoxy propanol (n-BP), butoxyethanol, or mixtures thereof. Suitable alkoxylated aromatic alcohols to be used herein are those according to the formula R¹²—(B)ᵣ—OH wherein R¹² is an alkyl substituted or non-alkyl substituted aryl group of from about 1 to about 20 carbon atoms, preferably from about 2 to about 15, and more preferably from about 2 to about 10, wherein B is an alkoxy group, preferably a butoxy, propoxy and/or ethoxy, and r is an integer of from 1 to 5, preferably from 1 to 2. A suitable aromatic alcohol to be used herein is benzyl alcohol. Suitable alkoxylated aromatic alcohol is benzylethanol and or benzylpropanol. Other suitable solvent includes butyl diglycolether, benzylalcohol, propoxypropoxypropanol (EP 0 859 044) ether and diether, glycol, alkoxylated glycol, C₆-C₁₆ glycol ether, alkoxylated aromatic alcohol, aromatic alcohol, aliphatic branched alcohol, alkoxylated aliphatic branched alcohol, alkoxylated linear C₁-C₅ alcohol, linear C₁-C₅ alcohol, amine, C₈-C₁₄ alkyl and cycloalkyl hydrocarbon and halohydrocarbon, and mixtures thereof.

### Perfume

The photocatalyzable consumer product composition of the present invention may comprise a perfume ingredient, or mixtures thereof, in amount up to about 5.0% by weight of the total composition, preferably in amount of about 0.1% to about 1.5%. Suitable perfume compounds and compositions for use herein are for example those described in EP-0 957 156.

### Dye

The photocatalyzable consumer product composition according to the present invention may be colored. Accordingly, it may comprise a dye or a mixture thereof. Suitable dyes for use herein are acid-stable dyes. By “acid-stable”, it is meant herein a compound which is chemically and physically stable in the acidic environment of the composition herein.

### pH Adjustment Agent

### Alkaline Material

An alkaline material may be present to trim the pH and/or maintain the pH of the composition according to the present invention. The amount of alkaline material is from about 0.001% to about 20%, preferably from about 0.01% to about 10%, and more preferably from about 0.05% to about 3% by weight of the composition.

Examples of the alkaline material are sodium hydroxide, potassium hydroxide and/or lithium hydroxide, and/or the alkali metal oxide, such as sodium and/or potassium oxide, or mixtures thereof. Preferably, the source of alkalinity is sodium hydroxide or potassium hydroxide, preferably sodium hydroxide.

### Acid

The photocatalyzable consumer product composition of the present invention may comprise an acid. Any acid known to those skilled in the art may be used herein. Typically the composition herein may comprise up to about 20%, preferably from about 0.1% to about 10%, more preferably from about 0.1% to about 5%, even more preferably from about 0.1% to about 3%, by weight of the total composition of an acid.

Suitable acids are selected from the group consisting of a mono- and poly-carboxylic acid or a mixture thereof; a percarboxylic acid or a mixture thereof; a substituted carboxylic acid or a mixture thereof; and mixtures thereof. Carboxylic acids useful herein include C₁₋₆ linear or at least about 3 carbon containing cyclic acids. The linear or cyclic carbon-containing chain of the carboxylic acid may be substituted with a substituent group selected from the group consisting of hydroxyl, ester, ether, aliphatic groups having from about 1 to about 6, more preferably from about 1 to about 4 carbon atoms, and mixtures thereof.

Suitable mono- and poly-carboxylic acids are selected from the group consisting of citric acid, lactic acid, ascorbic acid, isoascorbic acid, tartaric acid, formic acid, maleic acid, malic acid, malonic acid, propionic acid, acetic acid, dehydroacetic acid, benzoic acid, hydroxy benzoic acid, and mixtures thereof.

Suitable percarboxylic acids are selected from the group consisting of peracetic acid, percarbonic acid, perboric acid, and mixtures thereof.

Suitable substituted carboxylic acids are selected from the group consisting of an amino acid or a mixture thereof; a halogenated carboxylic acid or a mixture thereof; and mixtures thereof.

Preferred acids for use herein are selected from the group consisting of lactic acid, citric acid, and ascorbic acid and mixtures thereof. More preferred acids for use herein are selected from the group consisting of lactic acid and citric acid and mixtures thereof. An even more preferred acid for use herein is lactic acid.

Suitable acids are commercially available from JBL, T&L, or Sigma. Lactic acid is commercially available from Sigma and Purac.

### Salt

The photocatalyzable consumer product composition of the present invention may also comprise salts. When present, salts are generally used at a level of from about 0.01% to about 5%, preferably from about 0.015% to about 3%, more preferably from about 0.025% to about 2.0%, by weight of the composition.

When salts are included, the ions can be selected from magnesium, sodium, potassium, calcium, and/or magnesium, and preferably from sodium and magnesium, and are added as a hydroxide, chloride, acetate, sulphate, formate, oxide or nitrate salt to the composition of the present invention.

### Diamines

The photocatalyzable consumer product composition of the present invention may comprise a diamine or a mixture thereof as one or more components of the pH buffer. The composition will preferably contain from about 0% to about 15%, preferably from about 0.1% to about 15%, preferably from about 0.2% to about 10%, more preferably from about 0.25% to about 6%, more preferably from about 0.5% to about 1.5% by weight of the total composition of at least one diamine.

Preferred organic diamines are those in which pK₁ and pK₂ are in the range of from about 8.0 to about 11.5, preferably in the range of from about 8.4 to about 11, even more preferably from about 8.6 to about 10.75. Preferred materials include 1,3-bis(methylamine)cyclohexane (pKa=from about 10 to about 10.5), 1,3-propane diamine (pK₁=10.5; pK₂=8.8), 1,6-hexane diamine (pK₁=11; pK₂=10), 1,3-pentane diamine (DYTEK EP®) (pK₁=10.5; pK₂=8.9), 2-methyl-1,5-pentane diamine (DYTEK A®) (pK₁=11.2; pK₂=10.0). Other preferred materials include primary/primary diamines with alkylene spacers ranging from C₄ to C₈. In general, it is believed that primary diamines are preferred over secondary and tertiary diamines. pKa is used herein in the same manner as is commonly known to people skilled in the art of chemistry: in an all-aqueous solution at 25° C. and for an ionic strength between about 0.1 to about 0.5 M. values. Reference can be obtained from literature, such as from “Critical Stability Constants: Volume 2, Amines” by Smith and Martel, Plenum Press, NY and London, 1975.

### Chelants

The photocatalyzable consumer product composition of the present invention may include one or more chelants. When a chelant is used, a composition of the present invention may comprise a chelant at a level of from about 0.1% to about 20%, preferably from about 0.2% to about 5%, more preferably from about 0.2% to about 3% by weight of total composition.

Suitable chelants can be selected from the group consisting of an amino carboxylate or a mixture thereof; an amino phosphonate or a mixture thereof; a polyfunctionally-substituted aromatic chelant or a mixture thereof; and mixtures thereof.

Preferred chelants for use herein are the amino acid based chelants, and preferably glutamic-N,N-diacetic acid (GLDA) and derivatives, and/or phosphonate based chelants, and preferably diethylenetriamine pentamethylphosphonic acid. GLDA (salts and derivatives thereof) is especially preferred according to the invention, with the tetrasodium salt thereof being especially preferred.

Also preferred are amino carboxylates including ethylenediaminetetra-acetate, N-hydroxyethylethylenediaminetriacetate, nitrilo-triacetate, ethylenediamine tetrapro-prionate, triethylenetetraaminehexacetate, diethylenetriaminepentaacetate, ethanoldi-glycine; and alkali metal, ammonium, and substituted ammonium salts thereof; and mixtures thereof; as well as MGDA (methyl-glycine-diacetic acid), and salts and derivatives thereof.

Other chelants include homopolymers and copolymers of polycarboxylic acids and their partially or completely neutralized salts, monomeric polycarboxylic acids and hydroxycarboxylic acids and their salts. Preferred salts of the above-mentioned compounds are the ammonium and/or alkali metal salts, i.e. the lithium, sodium, and potassium salts, and particularly preferred salts are the sodium salts.

Suitable polycarboxylic acids are acyclic, alicyclic, heterocyclic and aromatic carboxylic acids, in which case they contain at least about two carboxyl groups which are in each case separated from one another by, preferably, no more than about two carbon atoms. Polycarboxylates which comprise two carboxyl groups include, for example, water-soluble salts of, malonic acid, (ethyl enedioxy)diacetic acid, maleic acid, diglycolic acid, tartaric acid, tartronic acid and fumaric acid. Polycarboxylates which contain three carboxyl groups include, for example, water-soluble citrate. Correspondingly, a suitable hydroxycarboxylic acid is, for example, citric acid. Another suitable polycarboxylic acid is the homopolymer of acrylic acid. Preferred are the polycarboxylates end capped with sulphonates.

Further suitable polycarboxylates chelants for use herein include acetic acid, succinic acid, formic acid; all preferably in the form of a water-soluble salt. Other suitable polycarboxylates are oxodisuccinates, carboxymethyloxysuccinate and mixtures of tartrate monosuccinic and tartrate disuccinic acid such as described in U.S. Pat. No. 4,663,071.

Amino phosphonates are also suitable for use as chelant and include ethylenediaminetetrakis (methylenephosphonates) as DEQUEST. Preferably, these amino phosphonates do not contain alkyl or alkenyl groups with more than about 6 carbon atoms. Polyfunctionally-substituted aromatic chelants are also useful in the composition herein, such as described in U.S. Pat. No. 3,812,044. Preferred compounds of this type in acid form are dihydroxydisulfobenzenes such as 1,2-dihydroxy-3,5-disulfobenzene.

### Hydrotrope

The photocatalyzable consumer product composition of the present invention may optionally comprise a hydrotrope in an effective amount so that the composition is appropriately compatible in water. The composition of the present invention typically comprises from about 0% to about 15% by weight of the total composition of a hydrotropic, or mixtures thereof, preferably from about 1% to about 10%, most preferably from about 3% to about 6%. Suitable hydrotropes for use herein include anionic-type hydrotropes, particularly sodium, potassium, and ammonium xylene sulphonate, sodium, potassium and ammonium toluene sulphonate, sodium potassium and ammonium cumene sulphonate, and mixtures thereof, and related compounds, as disclosed in U.S. Pat. No. 3,915,903.

### Polymeric Suds Stabilizer

The photocatalyzable consumer product composition of the present invention may optionally contain a polymeric suds stabilizer. These polymeric suds stabilizers provide extended suds volume and suds duration of the composition. The composition preferably contains from about 0.01% to about 15%, preferably from about 0.05% to about 10%, more preferably from about 0.1% to about 5%, by weight of the total composition of the polymeric suds booster/stabilizer.

These polymeric suds stabilizers may be selected from homopolymers of a (N,N-dialkylamino) alkyl ester and a (N,N-dialkylamino) alkyl acrylate ester. The weight average molecular weight of the polymeric suds booster, determined via conventional gel permeation chromatography, is from about 1,000 to about 2,000,000, preferably from about 5,000 to about 1,000,000, more preferably from about 10,000 to about 750,000, more preferably from about 20,000 to about 500,000, even more preferably from about 35,000 to about 200,000. The polymeric suds stabilizer can optionally be present in the form of a salt, either an inorganic or organic salt, for example the citrate, sulphate, or nitrate salt of (N,N-dimethylamino)alkyl acrylate ester.

One preferred polymeric suds stabilizer is (N,N-dimethylamino)alkyl acrylate ester, namely the acrylate ester represented by the formula (VII):

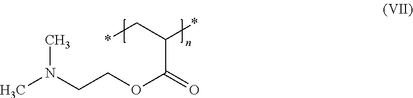

Other preferred suds boosting polymers are copolymers of hydroxypropylacrylate/dimethyl aminoethylmethacrylate (copolymer of HPA/DMAM), represented by the formulae VIII and IX

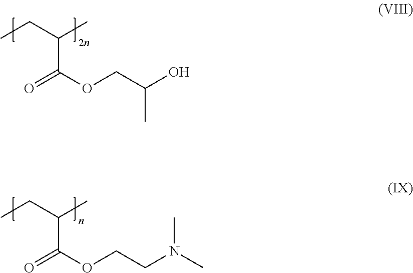

Another preferred class of polymeric suds booster polymers are hydrophobically modified cellulosic polymers having a weight average molecular weight (M_{w}) below about 45,000; preferably between about 10,000 and about 40,000; more preferably between about 13,000 and about 25,000. The hydrophobically modified cellulosic polymers include water soluble cellulose ether derivatives, such as nonionic and cationic cellulose derivatives. Preferred cellulose derivatives include methylcellulose, hydroxypropyl methylcellulose, hydroxyethyl methylcellulose, and mixtures thereof.

### Beauty Care Additives

It would be appreciated by one of ordinary skill in the art that many of the adjunct additives useful in any of the preceeding product types and beauty care products may generally fall into similar classes, though the specific preferred materials may differ.

The photocatalyzable consumer product composition may include one or more surfactants. Such surfactants can be suitable for application to skin and hair and are compatible with other components, including water. A photocatalyzable consumer product composition, in certain embodiments, can comprise from about 1% to about 30%, by weight of a surfactant or mixture of surfactants. In certain embodiments, the photocatalyzable consumer product composition can comprise from about 5% to about 25%, by weight of the photocatalyzable consumer product composition, of surfactants. In certain embodiments, the photocatalyzable consumer product composition can comprise from about 15% to about 22%, by weight of the photocatalyzable consumer product composition, of surfactants. The surfactants can include anionic (e.g. sodium laureth sulfate, ammonium lauryl sulfate and sodium trideceth sulfate), nonionic (e.g. isosteareth-2, trideceth-3, TDA-3), cationic, zwitterionic (e.g. cocoamidopropyl betaine), amphoteric surfactants (e.g. sodium lauroamphoacetate, sodium cocoamphoactetate and disodium lauroamphoacetate) and mixtures thereof. Suitable surfactants for the multiphase photocatalyzable consumer product composition are described in McCutcheon's: Detergents and Emulsifiers North American Edition (Allured Publishing Corporation 1947) (1986), McCutcheon's, Functional Materials North American Edition (Allured Publishing Corporation 1973) (1992) and U.S. Pat. No. 3,929,678 (filed Aug. 1, 1974).

In certain embodiments, the photocatalyzable consumer product composition can also be structured. In such embodiments that are structured, at least one surfactant (e.g., Sodium trideceth sulfate) may be included. In certain embodiments, a structured photocatalyzable consumer product composition can also can include from about 1% to about 20%, by weight of the photocatalyzable consumer product composition of surfactant; in certain embodiments from about 2% to about 15%, by weight of the photocatalyzable consumer product composition of surfactant; and in certain embodiments from about 5% to about 10%, by weight of the photocatalyzable consumer product composition of surfactant.

In a preferred embodiment, said surfactant may be sodium trideceth sulfate (STnS), where n is the average number of moles of ethoxylate per molecule. Trideceth is a 13 carbon branched ethoxylated hydrocarbon comprising, in one embodiment, an average of at least 1 methyl branch per molecule. In one embodiment, n can range from about 0 to about 3. In alternative embodiments, n can range from about 0.5 to about 2.7, from about 1.1 to about 2.5, from about 1.8 to about 2.2, or n can be about 2. When n can be less than 3, STnS can provide improved stability, improved compatibility of benefit agents within the photocatalyzable consumer product compositions, and increased mildness of the photocatalyzable consumer product compositions, such described benefits of STnS are disclosed in U.S. patent application Ser. No. 13/157,665.

The surfactant can also comprise one or more branched anionic surfactants and monomethyl branched anionic surfactants such as sodium tridecyl sulfate, sodium C₁₂-C₁₃ alkyl sulfate, and C₁₂-C₁₃ pareth sulfate and sodium C₁₂-C₁₃ pareth-n sulfate.

In a certain embodiment, the photocatalyzable consumer product composition can comprise at least one anionic surfactant, such as SLS. Suitable examples of SLS are described in U.S. patent application Ser. No. 12/817,786. In certain embodiments, the at least one surfactant can include sodium laureth(n) sulfate (hereinafter SLEnS), wherein n can define average moles of ethoxylation. In one embodiment, n can range from about 1 to about 3. Other suitable anionic surfactants can include ammonium lauryl sulfate, ammonium laureth sulfate, potassium laureth sulfate, sodium lauryl sarcosinate, sodium lauroyl sarcosinate, lauryl sarcosine, cocoyl sarcosine, ammonium cocoyl sulfate, potassium lauryl sulfate, and combinations thereof. However, in an alternative embodiment, the photocatalyzable consumer product composition can be optionally free of SLS, and can comprise at least a 70% lamellar structure.

As described above, in other embodiments, the surfactant can include nonionic and cationic surfactants. Nonionic surfactants for use in the photocatalyzable consumer product composition can include those selected from the group consisting of alkyl glucosides, alkyl polyglucosides, polyhydroxy fatty acid amides, alkoxylated fatty acid esters, sucrose esters, amine oxides, and mixtures thereof. Cationic surfactants for use in the photocatalyzable consumer product composition can include, but are not limited to, fatty amines, di-fatty quaternary amines, tri-fatty quaternary amines, imidazolinium quaternary amines, and combinations thereof.

Suitable amphoteric surfactants can include those that can be broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic surfactants suitable for use as described above can include those that are broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including cocoamidopropyl betaine. Additional suitable amphoteric or zwitterionic surfactants, in some embodiments, can include those described in U.S. Pat. Nos. 5,104,646 and 5,106,609.

A photocatalyzable consumer product composition can also include an associative and/or non-associative polymer. Associative polymers used in the cleansing phase can be a crosslinked, alkali swellable, associative polymer comprising acidic monomers and associative monomers with hydrophobic end groups, whereby the associative polymer comprises a percentage hydrophobic modification and a hydrophobic side chain comprising alkyl functional groups. Without intending to be limited by theory, it is believed the acidic monomers can contribute to an ability of the associative polymer to swell in water upon neutralization of acidic groups; and associative monomers anchor the associative polymer into structured surfactant hydrophobic domains, e.g., lamellae, to confer structure to the surfactant phase and keep the associative polymer from collapsing and losing effectiveness in a presence of an electrolyte. The crosslinked, associative polymer can comprise a percentage hydrophobic modification, which is a mole percentage of monomers expressed as a percentage of a total number of all monomers in a polymer backbone, including both acidic and other non-acidic monomers. Percentage hydrophobic modification of the associative polymer, hereafter % HM, can be determined by the ratio of monomers added during synthesis, or by analytical techniques such as proton nuclear magnetic resonance (NMR). Associative alkyl side chains can comprise, for example, butyl, propyl, stearyl, steareth, cetyl, lauryl, laureth, octyl, behenyl, beheneth, steareth, or other linear, branched, saturated, or unsaturated alkyl or alketh hydrocarbon side chains.

It has been discovered that crosslinked, associative polymers having certain % HM and certain carbon numbers of hydrophobic end groups of alkyl side chains can provide significant enhancement of structure to structured surfactant compositions, especially to compositions comprising reduced levels of surfactant. Such associative polymers can also provide the above structure at surprisingly low levels of polymer structurant. Concentrations of associative polymer of up to about 5% or even 10% are taught in the art to obtain a sufficient amount structure (e.g., exemplary compositions of U.S. Pat. No. 7,119,059 (Librizzi, et al.) and U.S. Pat. No. 6,897,253 (Schmucker-Castner, et al.). Inventors have found when associative polymer % HM and an alkyl side chain number of carbons can be optimized, structure of an aqueous structured surfactant phase can be increased using only less than about 3 wt %, less than about 2%, less than about 1%, and less than about 0.2%, of an associative polymer, as a percentage of an aqueous structured surfactant phase.

Acidic monomers can comprise any acid functional group, for example sulfate, sulfonate, carboxylate, phosphonate, or phosphate or mixtures of acid groups. In one embodiment, the acidic monomer comprises a carboxylate, alternatively the acidic monomer is an acrylate, including acrylic acid and/or methacrylic acid. The acidic monomer comprises a polymerizable structure, e.g., vinyl functionality. Mixtures of acidic monomers, for example acrylic acid and methacrylic acid monomer mixtures, are useful.

The associative monomer can comprise a hydrophobic end group and a polymerizable component, e.g., vinyl, which can be attached. The hydrophobic end group can be attached to the polymerizable component, hence to the polymer chain, by different means but can be attached by an ether or ester or amide functionality, such as an alkyl acrylate or a vinyl alkanoate monomer. The hydrophobic end group can also be separated from the chain, for example, by an alkoxy ligand such as an alkyl ether. In one embodiment, the associative monomer can be an alkyl ester, an alkyl(meth)acrylate, where (meth)acrylate is understood to mean either methyl acrylate or acrylate, or mixtures of the two.

In one embodiment, the hydrophobic end group of the associative polymer can be incompatible with the aqueous phase of the composition and can associate with lathering surfactant hydrophobe components. Without intending to be limited by theory, it is believed that longer alkyl chains of structuring polymer hydrophobe end groups can increase incompatibility with the aqueous phase to enhance structure, whereas somewhat shorter alkyl chains having carbon numbers closely resembling lathering surfactant hydrophobes (e.g., 12 to 14 carbons) or multiples thereof (for bilayers, e.g.) can also be effective. An ideal range of hydrophobic end group carbon numbers combined with an optimal percentage of hydrophobic monomers expressed as a percentage of the polymer backbone can provide increased structure to the lathering, structured surfactant composition at low levels of polymer structurant.

In one embodiment, the associative polymer is AQUPEC® SER-300 made by Sumitomo Seika of Japan, which is an acrylate/C₁₀-C₃₀ alkyl acrylate cross-polymer and comprises stearyl side chains with less than about 1% HM. Associative polymers can comprise about C₁₆ (cetyl) alkyl hydrophobic side chains with about 0.7% hydrophobic modification, but a percentage hydrophobic modification can be up to an aqueous solubility limit in surfactant compositions (e.g., up to 2%, 5%, or 10%). Other associative polymers can include stearyl, octyl, decyl and lauryl side chains, alkyl acrylate polymers, polyacrylates, hydrophobically-modified polysaccharides, hydrophobically-modified urethanes, AQUPEC® SER-150 (acrylate/C₁₀-C₃₀ alkyl acrylate cross-polymer) comprising about C₁₈ (stearyl) side chains and about 0.4% HM, and AQUPEC® HV-701EDR which comprises about C₈ (octyl) side chains and about 3.5% HM, and mixtures thereof. In another embodiment, the associative polymer can be Stabylen 30 manufactured by 3V Sigma S.p.A., which has branched isodecanoate hydrophobic associative side chains.

The photocatalyzable consumer product composition can further include a non-associative polymer. Suitable non-associative polymers can include water-dispersible polymers with relatively uniform hydrophilic backbone lacking hydrophobic groups. Examples of non-associative polymers can include biopolymer polysaccharides (e.g., xanthan gum, gellan gum), cellulosic polysaccharides (e.g., carboxymethyl cellulose, carboxymethyl hydroxyethyl cellulose), other polysaccharides (e.g., guar gum, hydroxypropyl guar, and sodium alginate), and synthetic hydrocarbon polymers (e.g., polyacrylamide and copolymers, polyethylene oxide, polyacrylic acid copolymers).

Photocatalyzable consumer product composition can additionally comprise an organic cationic deposition polymer in one or more phases as a deposition aid for benefit agents described herein. Suitable cationic deposition polymers can contain cationic nitrogen-containing moieties such as quaternary moieties. Non-limiting examples of cationic deposition polymers can include polysaccharide polymers, such as cationic cellulose derivatives. Cationic cellulose polymers can be salts of hydroxyethyl cellulose reacted with trimethyl ammonium substituted epoxide, referred to in the industry (CTFA) as Polyquaternium 10, which can be available from Amerchol Corp. (Edison, N.J.) in their Polymer KG, JR, and LR series of polymers. Other suitable cationic deposition polymers can include cationic guar gum derivatives, such as guar hydroxypropyltrimonium chloride, specific examples of which can include the Jaguar series commercially available from Rhodia Inc. and N-Hance polymer series commercially available from Aqualon. Suitable water-soluble cationic deposition polymers can include synthetic polyacrylamides such as Polyquaternium 76 and Polymethylene-bis-acrylamide methacrylamido propyltrimethyl ammonium chloride (PAM/MAPTAC). Such PAM/MAPTAC can have an acrylamide to methacrylamido propyltrimethyl ammonium chloride ratio of 88:12. In one embodiment, the deposition polymers can have a cationic charge density from about 0.8 meq/g to about 2.0 meq/g; and in certain embodiments from about 1.0 meq/g to about 1.5 meq/g.

A photocatalyzable consumer product composition can also include water. In one embodiment, the photocatalyzable consumer product composition can comprise from about 10% to about 90%, by weight of the photocatalyzable consumer product composition, of water; in certain embodiments, from about 40% to about 85%, by weight of the photocatalyzable consumer product composition, of water, and in certain embodiments, from about 60% to about 80%, by weight of the photocatalyzable consumer product composition, of water.

Other optional additives can be included in the consumer product phase, including for example an emulsifier (e.g., non-ionic emulsifier) and electrolyes. Suitable electrolytes can includes an anion such as phosphate, chloride, sulfate, citrate, and mixtures thereof and a cation such as sodium, ammonium, potassium, magnesium, and mixtures thereof. For example, suitable electrolytes can include sodium chloride, ammonium chloride, sodium sulfate, ammonium sulfate, and mixtures thereof. Other suitable emulsifiers and electrolytes are described in U.S. patent application Ser. No. 13/157,665.

### Non-Photocatalyzable Benefit Agents

A photocatalyzable consumer product composition can also include one or more non-photocatalyzable benefit agents. In particular, the photocatalyzable consumer product composition can comprise from about 0.1% to about 50%, by weight of the photocatalyzable consumer product composition, of a non-photocatalyzable benefit agent. In certain embodiments, the photocatalyzable consumer product composition can include from about 0.5% to about 20%, by weight of the photocatalyzable consumer product composition, of the non-photocatalyzable benefit agent; and in certain embodiments, the photocatalyzable consumer product composition can include from about 1.0% to about 10%, by weight of the photocatalyzable consumer product composition, of the non-photocatalyzable benefit agent. Such non-photocatalyzable benefit agents can include hydrophobic benefit agents.

Non-limiting examples of non-photocatalyzable benefit agents can include petrolatum, silicones, glyceryl monooleate, mineral oil, natural oils (e.g., soybean oil, saturated or unsaturated), sucrose esters, cholesterol, fatty esters, fatty alcohols, petrolatum, glyceryl monooleate, zinc phrithione, Olivem 1000, and mixtures thereof. Other suitable non-photocatalyzable benefit agents are described in U.S. patent application Ser. No. 13/157,665.

Additional non-limiting examples of non-photocatalyzable benefit agents include SEFOSE®, lanolin esters, lanolin oil, natural waxes, synthetic waxes, volatile organosiloxanes, derivatives of volatile organosiloxanes, non-volatile organosiloxanes, derivatives of non-volatile organosiloxanes, natural triglycerides, synthetic triglycerides, polyglycerides, and combinations thereof.

In another embodiment, a non-photocatalyzable benefit agent comprises a metathesized unsaturated polyol ester. A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds (i.e., olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I:

R¹—CH═CH—R²+R¹—CH═CH—R² [symbol] R¹—CH═CH—R¹+R²—CH═CH—R² (I)

where R¹ and R² are organic groups.

Cross-metathesis may be represented schematically as shown in Equation II:

R¹—CH═CH—R²+R³—CH═CH—R⁴ [symbol] R¹—CH═CH—R³+R—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴ (II)

where R¹, R², R³, and R⁴ are organic groups.

When the unsaturated poyol ester comprises molecules that have more than one carbon-carbon double bond (i.e., a polyunsaturated polyol ester), self-metathesis results in oligomerization of the unsaturated polyol ester. The self-metathesis reaction results in the formation of metathesis dimers, metathesis trimers, and metathesis tetramers. Higher order metathesis oligomers, such as metathesis pentamers and metathesis hexamers, may also be formed by continued self-metathesis and will depend on the number and type of chains connecting the unsaturated polyol ester material as well as the number of esters and orientation of the ester relative to the unsaturation.

As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. In many embodiments, the unsaturated polyol ester can be represented by the general structure I:

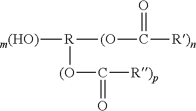

where n≧1; m≧0; p≧0; (n+m+p)≧2; R is an organic group; R is an organic group having at least one carbon-carbon double bond; and R is a saturated organic group. Exemplary embodiments of the unsaturated polyol ester are described in detail in U.S. 2009/0220443 A1.

In many embodiments of the invention, the unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of theses, and the like. Recycled used vegetable oils may also be used. Representative examples of vegetable oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high oloeyl sunflower oil, high oleoyl soybean oil, high erucic rape oils, Jatropha oil, combinations of theses, and the like. Representative examples of animal fats include lard, tallow, chicken fat, yellow grease, fish oil, combinations of these, and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Sugar esters such as SEFOSE® include one or more types of sucrose polyesters, with up to eight ester groups that could undergo a metathesis exchange reaction.

SEFOSE® includes one or more types of sucrose polyesters. Sucrose polyesters are derived from a natural resource and therefore, the use of sucrose polyesters as the benefit agents can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. In one embodiment the sucrose polyester may have an IBAR of from about 5 to about 8. In another embodiment the sucrose polyester has an IBAR of about 5-7, and in another embodiment the sucrose polyester has an IBAR of about 6. In yet another embodiment the sucrose polyester has an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140. In another embodiment the sucrose polyester of the present invention may have an IV of about 10 to about 120. In yet another embodiment the sucrose polyester of the present invention may have an IV of about 20 to 100. Further, such sucrose polyesters have a chain length of about C₁₂ to C₂₀.

Non-limiting examples of sucrose polyesters suitable for use include sucrose polysoyate (SEFOSE® 1618S), SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, sucrose polycottonseedate, SEFOSE® C1295, Sefa C895, Sefa C1095, SEFOSE® 1618S B4.5, all available from The Procter and Gamble Co. of Cincinnati, Ohio.

Non-limiting examples of glycerides suitable for use as hydrophobic skin benefit agents herein can include castor oil, safflower oil, corn oil, walnut oil, peanut oil, olive oil, cod liver oil, almond oil, avocado oil, palm oil, sesame oil, soybean oil, unsaturated soybean oil, vegetable oils, sunflower seed oil, vegetable oil derivatives, coconut oil and derivatized coconut oil, cottonseed oil and derivatized cottonseed oil, jojoba oil, cocoa butter, and combinations thereof.

Non-limiting examples of alkyl esters suitable for use as hydrophobic skin benefit agents herein can include isopropyl esters of fatty acids and long chain esters of long chain (i.e. C₁₀-C₂₄) fatty acids, e.g., cetyl ricinoleate, non-limiting examples of which can include isopropyl palmitate, isopropyl myristate, cetyl riconoleate, and stearyl riconoleate. Other examples can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic skin benefit agents herein can include oleyl myristate, oleyl stearate, oleyl oleate, and combinations thereof.

Non-limiting examples of polyglycerin fatty acid esters suitable for use as hydrophobic skin benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic skin benefit agents herein can include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Non-limiting examples of silicone oils suitable for use as hydrophobic skin benefit agents herein can include dimethicone copolyol, dimethylpolysiloxane, diethylpolysiloxane, mixed C₁-C₃₀ alkyl polysiloxanes, phenyl dimethicone, dimethiconol, and combinations thereof. Non-limiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

In such embodiments where the hydrophobic benefit agent can include SEFOSE®, hydrophobic benefit agents can exhibit a total surface energy of about 20 mJ/m² to about 30 mJ/m² and a change in heat of fusion (or A heat of fusion) of about 50 J/g or less, which provide for favorable deposition. As shown in FIG. 4, sucrose polyesters having a total surface energy of about 20 mJ/m² to about 30 mJ/m², a range comparable to that of skin in the presence of a surfactant, can be thermodynamically favorable for deposition such that the similar surface energies can promote adhesion and spreading. Further, using a A heat of fusion of about 50 J/g or less can ensure that the sucrose polyester can be fluid-like for adequate coverage and spreading. Such properties can be favorable for enhanced chroma measurements because, and as described herein, chroma of the skin can be increased through increased deposition of the hydrophobic benefit agent.

In certain embodiments, such hydrophobic benefit agents as described herein can be combined with a soy oligomer. In certain embodiments, the photocatalyzable consumer product composition can include from about 1% to about 50%, by weight of the photocatalyzable consumer product composition, of a soy oligomer; in certain embodiments, from about 2% to about 40%, by weight of the photocatalyzable consumer product composition, of a soy oligomer; in certain embodiments, from about 3% to about 30%, by weight of the photocatalyzable consumer product composition, of a soy oligomer; in certain embodiments, from about 4% to about 20%, by weight of the photocatalyzable consumer product composition, of a soy oligomer; and in certain embodiments, from about 5% to about 15%, by weight of the photocatalyzable consumer product composition, of a soy oligomer. In certain embodiments, the soy oligomer can be fully or partially hydrogenated. For example, in certain embodiments, the soy oligomer can be about 60% hydrogenated or more; in certain embodiments, about 70% hydrogenated or more; in certain embodiments about 80% hydrogenated or more; in certain embodiments, about 85% hydrogenated or more; in certain embodiments, about 90% hydrogenated or more; and in certain embodiments, generally 100% hydrogenated.

Soy oligomers can be prepared by the metathesis of soybean oil with a metal catalyst, followed by hydrogenation. The soy oligomer can include hydrogenated soy polyglycerides. The soy oligomer may also include C₁₅-C₂₃ alkanes, as a byproduct. In particular, the soy oligomer can be DOW CORNING® HY-3050 soy wax or DOW CORNING® HY-3051 soy wax blend, both available from Dow Corning. Other oligomers can also be formed using metathesized unsaturated polyol ester (e.g., metathesized vegetable oil). Such other examples include metathesized canola oil, metathesized rapeseed oil, metathesized coconut oil, metathesized corn oil, metathesized cottonseed oil, metathesized olive oil, metathesized palm oil, metathesized peanut oil, metathesized safflower oil, metathesized sesame oil, metathesized sunflower oil, metathesized linseed oil, metathesized palm kernel oil, metathesized tung oil, and metathesized castor oil. In other embodiments, the metathesized unsaturated polyol ester is a metathesized animal fat, for example, metathesized lard, metathesized tallow, metathesized chicken fat (i.e., yellow grease), and metathesized fish oil. Mixtures of the foregoing may also be useful. Such suitable examples are described in U.S. Patent Application Publication No. 2009/0220443.

In certain embodiments, a hydrophobic benefit agent can exhibit a Vaughan solubility parameter from about 5 to about 14 and exhibit a viscosity of about 1500 cP or less at from about 20° C. to about 25° C. In certain embodiments, a hydrophobic benefit agent can exhibit a Vaughan solubility parameter from about 7 to about 12 and exhibit a viscosity of about 1500 cP or less at from about 20° C. to about 25° C. Vaughan solubility parameters are defined in Vaughan in Cosmetics and Toiletries, Vol. 103. Non-limiting examples of hydrophobic materials having Vaughan solubility parameter values in the above range can include the following: Cyclomethicone, 5.92; Squalene, 6.03; Petrolatum, 7.33; Isopropyl Palmitate, 7.78; Isopropyl Myristate, 8.02; Castor Oil, 8.90; Cholesterol, 9.55; as reported in Solubility, Effects in Product, Package, Penetration and Preservation, C. D. Vaughan, Cosmetics and Toiletries, Vol. 103, October 1988.

Optional ingredients and/or actives can also be added to the photocatalyzable consumer product composition for treatment of the skin, or to modify the aesthetics of the photocatalyzable consumer product composition as is the case with perfumes, colorants, dyes or the like. However, in certain embodiments, the photocatalyzable consumer product composition is free of any pigments, colorants, or dyes. Optional materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these optional ingredients and/or actives, and levels of incorporation thereof, will depend on the physical form of the composition and the nature of the cleansing operation for which it is to be used. Optional ingredients and/or actives can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less by weight of the photocatalyzable composition.

Other non-limiting optional ingredients that can be used in the photocatalyzable consumer product composition can include an optional benefit component that can be selected from the group consisting of thickening agents; preservatives; antimicrobials; fragrances; chelators (e.g., such as those described in U.S. Pat. No. 5,487,884 issued to Bisset, et al.); sequestrants; vitamins (e.g., Retinol); vitamin derivatives (e.g., tocophenyl actetate, niacinamide, panthenol); sunscreens; desquamation actives (e.g., such as those described in U.S. Pat. Nos. 5,681,852 and 5,652,228 issued to Bisset); anti-wrinkle/anti-atrophy actives (e.g., N-acetyl derivatives, thiols, hydroxyl acids, phenol); anti-oxidants (e.g., ascorbic acid derivatives, tocophenol) skin soothing agents/skin healing agents (e.g., panthenoic acid derivatives, aloe vera, allantoin); skin lightening agents (e.g., kojic acid, arbutin, ascorbic acid derivatives) skin tanning agents (e.g., dihydroxyacteone); anti-acne medicaments; essential oils; sensates; pigments; colorants; pearlescent agents; interference pigments (e.g., such as those disclosed in U.S. Pat. No. 6,395,691 issued to Liang Sheng Tsaur, U.S. Pat. No. 6,645,511 issued to Aronson, et al., U.S. Pat. No. 6,759,376 issued to Zhang, et al, U.S. Pat. No. 6,780,826 issued to Zhang, et al.) particles (e.g., talc, kolin, mica, smectite clay, cellulose powder, polysiloxane, silicas, carbonates, titanium dioxide, polyethylene beads) hydrophobically modified non-platelet particles (e.g., hydrophobically modified titanium dioxide and other materials described in a commonly owned, patent application published on Aug. 17, 2006 under Publication No. 2006/0182699A, entitled “Personal Care Compositions Containing Hydrophobically Modified Non-platelet particle filed on Feb. 15, 2005 by Taylor, et al.) and mixtures thereof. In one embodiment, the photocatalyzable consumer product composition can comprise from about 0.1% to about 4%, by weight of the photocatalyzable consumer product composition, of hydrophobically modified titanium dioxide. Other such suitable examples of such skin actives are described in U.S. patent application Ser. No. 13/157,665.

### METHODS OF USE

The present invention further relates to methods of using the compositions of the present invention to provide benefits such as cleaning surfaces, bleaching stains (including whitening teeth) disinfecting and/or sanitizing surfaces, removing biofilm from surfaces, and the like.

As such, the present invention encompasses a method of cleaning a surface, the method comprising the steps of contacting the surface with a consumer product composition of the present invention and exposing the consumer product composition to light, preferably having a wavelength greater than about 350 nm. The light utilized can be from a natural or artificial source.

The present invention further encompasses a method of bleaching a stain, the method comprising the steps of contacting the stain with a consumer product composition of the present invention and exposing the consumer product composition to light, preferably having a wavelength greater than about 350 nm.

The present invention further encompasses a method of disinfecting a surface, the method comprising the steps of contacting the surface with a consumer product composition of the present invention and exposing the consumer product composition to light, preferably having a wavelength greater than about 350 nm.

The present invention further encompasses a method of removing biofilm from a surface, the method comprising the steps of contacting the biofilm with a consumer product composition of the present invention and exposing the consumer product composition to light, preferably having a wavelength greater than about 350 nm.

The present invention also relates to a method for cleaning a stained fabric comprising contacting a stained fabric in need of cleaning with the photocatalyzable consumer product composition, described in detail above, having at least 0.001 ppm of a photoactivator, described in detail above, followed by exposing the surface of the treated fabric to a source of light having a minimal wavelength range of greater than about 300 nanometers, preferably greater than about 350 nanometers, preferably greater than about 400 nm, up to about 550 nanometers, preferably up to about 500 nanometers.

The present invention further relates to a method for cleaning a surface comprising contacting a surface in need of cleaning with the photocatalyzable consumer product composition, described in detail above, having at least 0.001 ppm of a photoactivator, described in detail above, followed by exposing the surface to a source of light having a minimal wavelength range of greater than about 300 nanometers, preferably greater than about 350 nanometers, up to about 550 nanometers, preferably up to about 500 nanometers.

The present invention further relates to a method for treating or cleaning oral cavity, including teeth or dentures (inside or outside the oral cavity), comprising contacting the oral cavity (including teeth or dentures) in need of treatment or cleaning with the photocatalyzable consumer product composition, described in detail above, having at least 0.001 ppm of a photoactivator, described in detail above, followed by exposing the teeth or dentures to a source of light having a minimal wavelength range of greater than about 300 nanometers, preferably greater than about 350 nanometers, up to about 550 nanometers, preferably up to about 500 nanometers.

### Packaging

The photocatalyzable consumer product compositions of the present invention may be packed in any suitable packaging for delivering the photocatalyzable consumer product compositions for use. It will be understood, however, that the package may be structured to prevent the photoactivator from absorbing light and, therefore, activation of the benefit active before use. In one aspect, the package can be opaque. In another aspect, the package can be a transparent or translucent package made of glass or plastic so that consumers can see the photocatalyzable consumer product compositions throughout the packaging. In another aspect, the package may include one or more windows which may be opened to allow the consumer to see the composition and/or activate the composition prior to use and subsequently closed to prevent the photoactivator from absorbing light during storage. In one preferred aspect, the package may be comprised of polyethylene terephthalate, high-density polyethylene, low-density polyethylene, or combinations thereof. Furthermore, preferably, the package may be dosed through a cap at the top of the package such that the composition exits the bottle through an opening in the cap. In one aspect, the opening in the cap may also contain a screen to help facilitate dosing.

### Chlorite Quenching Test Method

The photoactivators of the present invention are evaluated for suitability by the following process.

A suitable wavelength for excitation of the photoactivator is determined by recording a UV/Vis spectrum on any suitable UV/Vis spectrophotometer and identifying an absorption band in the range from 350 nm to 750 nm.

The steady state fluorescence is first determined using a Fluorolog 3 (model number FL3-22) fluorescence spectrophotometer from Horiba Jobin Yvon to acquire the fluorescence spectrum of the photoactivator. It will be understood by those skilled in the art that the fluorescence produced by the activator varies depending on the fluorescence quantum yield for the structure. The photoactivators are screened through a wide range of concentrations (1 ppm-10,000 ppm) to determine the concentration which produces the approximate maximum steady state fluorescence.

Fluorescence quenching is demonstrated by producing solutions of the photoactivator at the concentration determined as described above with a range of concentrations of sodium chlorite (1000 ppm-100,000 ppm).

Photoactivators of the present invention are considered suitable if steady state fluorescence is reduced at least 10% (based on counts per second) when the photoactivator is dissolved in a 1% solution of sodium chlorite.

### Indigo Carmine Bleaching Test Method

Photoactivators that demonstrate reduced steady state fluorescence in the presence of chlorite are evaluated for the generation of the benefit active chlorine dioxide. A solution of the activator (at the above described concentration) is prepared in 1% aqueous sodium chlorite containing 20 ppm indigo carmine as a bleaching indicator.

The solution is exposed to light at the excitation wavelength for the generation of the excited state of the photoactivator and a UV/Vis spectra taken after ten minutes of light exposure. The reduction in the intensity of the indigo carmine visible absorption peak is used to determine the bleaching efficacy of the photoactivator in the presence of sodium chlorite. Photoactivators of the present invention are considered suitable if the Indigo carmine absorption peak intensity was reduced by more than a control solution that does not contain chlorite.

### EXAMPLES

### Photoactivator Examples

The following are non-limiting examples of various water soluble organic photoactivators, and syntheses thereof, of the present invention.

### 9-Oxo-9H-thioxanthene-2-carboxylic acid chloride

A dry 500 mL 1-neck recovery flask containing 13.25 g of 9-oxo-9H-thioxanthene-2-carboxylic acid and a magnetic stir bar is fitted with a dry condenser connected to Firestone valve (with the bubbler exit going through water to trap evolved HCl). After adding 250 mL of thionyl chloride the system is vacuum/nitrogen cycled 5 times and left under positive nitrogen pressure (suspended solid). After refluxing for 5 hours thionyl chloride is removed in vacuo using a rotary evaporator at 60° C. The residual solid on the flask walls is scraped down and broken up and placed under vacuum overnight (0.3 mm Hg) at room temperature. The vacuum is broken while introducing argon and the solid is broken up using a glass rod and spatula while maintaining a flow of argon over the mouth of the flask. The overnight vacuum treatment is repeated leading to 11.92 g of pinkish solid acid chloride.

### Thioxanthenone-PEG(10,000) Ester Conjugate

Using oven-dried glassware 434.0 g poly(ethylene glycol) (MW 10,000) is placed in a 3 L 3-neck round-bottom flask with mechanical stirrer, condenser (topped with nitrogen/vacuum inlet), and a Teflon thermocouple connected to temperature controller and heating mantle. The system is cycled between nitrogen and vacuum and left under nitrogen.

The addition of 0.64 g of 4-(dimethylamino)pyridine and 6.3 mL of triethylamine is followed by the addition of 500 mL of anhydrous methylene chloride. The system is cycled between nitrogen and vacuum and left under nitrogen again as the mixture is stirred to dissolve the materials. A suspension of 11.92 g 9-oxo-9H-thioxanthene-2-carboxylic acid chloride in 1160 mL of anhydrous methylene chloride is transferred into the reaction mixture. The system is cycled between nitrogen and vacuum and left under nitrogen again as the pink solid suspension mixture soon became opaque and tan. After stirring at ambient for 3 hours the mixture is stirred an additional 48 hours at 40° C. The reaction mixture is extracted twice with 100 mL of a pH 3 aqueous solution (prepared by mixing 2 parts of saturated aqueous sodium chloride and 1 part water and adjusting the pH with 0.1 N hydrochloride acid). The resulting emulsions required about an hour to separate. After washing the organic phase with 300 mL of saturated aqueous sodium chloride solution it is dried over 300 g of sodium sulfate overnight. After suction filtering the solvent is removed in vacuo using a rotary evaporator to give 379.9 g of yellowish solid which is scraped from the flask and ground up with a mortar and pestle. The ground up solid is placed under 0.18 mm Hg of vacuum overnight before mixing with 1600 mL of water. This cloudy solution is suction filtered through two glass fiber pads to give 1817.2 g of a yellow-green aqueous solution found to be 19.0 weight percent solids after freeze-drying a portion of it. The resulting photoactivator exhibits a suitable excitation wavelength of about 380 nm and comprises about 2%, by weight of the photoactivator, of photoactive moiety.

### Anthraquinone-mPEG(550) Ester Conjugate

A 100 mL round-bottom flask containing 5.08 g of poly(ethylene glycol) methyl ether (mPEG-550; Mₙ ca 550, Tₘ=20° C.), 0.113 g of 4-(dimethylamino)pyridine, 1.4 mL of triethylamine, 40 mL of methylene chloride, and a magnetic stir bar is fitted with a condenser connected to a Firestone valve (for vacuum and nitrogen introduction). While stirring under nitrogen 2.50 g anthraquinone-2-carbonyl chloride is added at room temperature and then the mixture is heated to reflux for 48 hours. After cooling and adding an additional 50 mL of methylene chloride the mixture is extracted with 50 mL of 1M HCl and twice with 50 mL of water. The organic solution is dried over magnesium sulfate. After suction filtering the solvent is removed in vacuo at 45° C. using a rotary evaporator. The light beige solid residue is taken up in 115 mL of water to provide a turbid solution which is suction filtered through a glass fiber pad under a paper filter pad. Freeze-drying led to 4.1 g of a sticky beige solid which is dissolved to make a 10 wt. % aqueous solution. The resulting photoactivator exhibits a suitable excitation wavelength of about 450 nm and comprises about 27%, by weight of the photoactivator, of photoactive moiety.

### Anthraquinone-mPEG(2000) Ester Conjugate

A 100 mL round-bottom flask containing 18.47 g of poly(ethylene glycol) methyl ether (mPEG-2000, 1.500 g; Mₙ ca 2000, Tₘ=52° C.), 0.112 g of 4-(dimethylamino)pyridine, 1.4 mL of triethylamine, 105 mL of methylene chloride, and a magnetic stir bar is fitted with a condenser connected to a Firestone valve (for vacuum and nitrogen introduction). While stirring under nitrogen 2.50 g anthraquinone-2-carbonyl chloride is added at room temperature and then the mixture is heated to reflux for 48 hours. After cooling and adding an additional 50 mL of methylene chloride the mixture is extracted with 50 mL of 1M HCl and twice with 50 mL of water. The organic solution is dried over magnesium sulfate. After suction filtering the solvent is removed in vacuo at 45° C. using a rotary evaporator. The light beige solid (16.66 g) residue is taken up in 666 mL of water to provide a turbid solution which is suction filtered through a glass fiber pad under a paper filter pad. Freeze-drying led to 12.75 g of a light yellow solid which is dissolved to make a 10 wt. % aqueous solution. The resulting photoactivator exhibits a suitable excitation wavelength of about 435 nm and comprises about 9%, by weight of the photoactivator, of photoactive moiety.

### Gantrez-Naphthylmethyl Amide Conjugate

A 250 mL round-bottom flask containing 5.075 g of Gantrez (anhydride form; M_{w}216,000; Mₙ 80,000), 125 mL of tetrahydrofuran, and a magnetic stir bar is fitted with a condenser connected to a Firestone valve (for vacuum and nitrogen introduction) then stirred and heated to reflux under nitrogen. The polymer partially dissolved. After cooling to room temperature 1.32 g of triethylamine is added leading to some solids coming out of solution with the development of a light purple color. The addition of 1.02 g of 1-naphthylenemethylamine led to a darker purple color and the mixture is stirred at room temperature under nitrogen for 26 hours. An aqueous solution of 1.0 N sodium hydroxide (58.5 mL) is slowly added to reaction and the mixture is stirred another 17 hours at room temperature. The two-phase mixture is transferred to a 1 L flask with 100 mL of water and concentrated at 50° C. in vacuo using a rotary evaporator. Three additional cycles were performed adding 100 mL of water and concentrating to give 5.22 g of a tan/yellow solid. This residue is taken up in 105 mL of water, suction filtered, and the filtrate is freeze-dried to provide 7.41 g of a light solid which is diluted to a 5 wt % aqueous solution. The resulting photoactivator exhibits a suitable excitation wavelength of about 405 nm and comprises about 11%, by weight of the photoactivator, of photoactive moiety.

### 2-(2-Aminoethyl)-1H-benzo[de]isoquinoline-1,3(2H)-dione

A 100 mL round-bottom flask is charged with 20 mL of ethylene diamine along with a magnetic stir bar. A slurry of 5.00 g of 1,8-naphthalic anhydride and 30 mL of pyridine is added to the ethylene diamine, the flask is fitted with an air condenser, and the resulting slurry is stirred and heated to 60° C. under an argon atmosphere for 23 hours and an additional 24 hours at room temperature. The reaction mixture is then poured into 350 mL of water stirring in 1 L beaker and the resulting solid is suction filtered through #4 filter paper and washed with 3×40 mL of water on the funnel. The filtered solid is dried under vacuum (0.3 mm Hg) for 6 hours to give 3.767 g of an off-white powder, 2-(2-aminoethyl)-1H-benzo[de]isoquinoline-1,3(2H)-dione.

### Gantrez-Naphthylene Amide Conjugate

A 500 mL round-bottom flask containing 3.00 g of Gantrez (anhydride form; M_{w}216,000; Mₙ 80,000), 75 mL of tetrahydrofuran, and a magnetic stir bar is fitted with an air condenser connected to a Firestone valve (for vacuum and nitrogen introduction) then stirred and heated to 60° C. under argon. The polymer dissolved to give a homogeneous solution. After cooling to room temperature 1.1 mL of triethylamine is added leading to some solids coming out of solution with the development of a reddish color. After cooling to room temperature 0.924 g of 2-(2-aminoethyl)-1H-benzo[de]isoquinoline-1,3(2H)-dione is added, the system is purged with argon again, and then stirred at 60° C. for 20 hours. The freely stirring purple solution with some suspended solids is cooled to room temperature and 35 mL of 1.0 N NaOH is added leading to the precipitation of a gummy, brown material. After 2.5 hours at room temperature 50 mL of methanol is added and stirring is continued at room temperature under argon overnight. The mixture with the insoluble gummy material is then heated to a gentle reflux for 4 hours before concentrating in vacuo using a rotary evaporator at 55° C. After the addition 200 mL of water and subsequent concentration the residue is agitated with 300 mL of water at 55° C. Most of the residue dissolved and the hazy solution is suction filtered while warm on a 90 mm Buchner funnel through layers of #4 filter paper/glass fiber pad/#4 filter paper resulting in a clear (tannish) solution. Freeze-drying led to 6.0 g of an off-white foam which is dissolved in water to make a 0.026 g/mL solution.

### 1-Naphthoyl-4-mPEG semicarbazide conjugate

Methoxypoly(ethylene glycol) isocyanate (1.025 g; MW-2000) is placed in a 10 mL round-bottom flask with a magnetic stir bar and dissolved in 2 mL of methylene chloride. While stirring a suspension of 0.186 g of 1-naphthoic hydrazide in 2 mL of methylene chloride is added, the flask is capped and covered with foil to protect from light, and stirring is continued at room temperature. After 6 days solvent is removed in vacuo using a rotary evaporator to give 1.90 g of white solid. This material is dissolved/suspended in 100 mL of water. This solution is filtered through #3 filter paper to clarify and freeze-dried to provide 1.09 g of fluffy white solid with a pinkish tint. This material is redissolved in a total volume of 100 mL of water to give a 0.0107 g/mL aqueous solution.

### Naphthylenemethyl-mPEG(2000) Urea Conjugate

Methoxypoly(ethylene glycol) isocyanate (1.025 g; MW˜2000) is placed in a 10 mL round-bottom flask with a magnetic stir bar and dissolved in 2 mL of methylene chloride. While stirring 0.184 g of 1-naphthylenemethylamine is added, the flask is capped and covered with foil to protect from light, and stirring is continued at room temperature. After 4 days solvent is removed in vacuo using a rotary evaporator to give 1.12 g of white solid. This material is dissolved in 50 mL of water to make a hazy homogenous solution (pH 7). This solution is filtered through #3 filter paper to clarify, and then diluted with water to a total volume of 70 mL. A portion of the solution is freeze-dried to determine that the solution had a concentration of 0.0133 g/mL.

### Starch-Naphthalene Carbamate Conjugate

A solution of 0.676 g of 1-naphthyl isocyanate in 13 mL of THF is added to 3.24 g of starch (Aldrich catalog number 85652) in a 100 mL round-bottom flask with a magnetic stir bar. The flask is fitted with an air condenser and heated to 60° C. for 3 days under argon. The mixture is concentrated under reduced pressure (rotary evaporator) at 40° C., slurried in 100 mL of water, concentrated again, and repeated. The resulting residue is slurried in 300 mL of water, heated on a steam bath, then centrifuged to separate from most of the solids. The aqueous solution is then suction filtered and freeze-dried to give 0.70 g of white, fibrous solid. A in 0.307 g portion of this solid is suspended in 300 mL of water (with steam heating), let cool to room temperature, then let sit overnight to let solids settle out. The aqueous solution is then suction filtered and diluted to 300 mL. A portion of this solution is freeze-dried to determine that the solution had a concentration of 0.00083 g/mL. The resulting photoactivator exhibits a suitable excitation wavelength of about 330 nm.

mPEG(2000)-Naphthalic Anhydride Conjugate

Solid reagents, 0.300 g of 1,8-naphthalic anhydride and 3.90 g of poly(ethylene glycol) methyl ether (mPEG-2000, 1.500 g; Mₙ ca 2000, Tₘ=52° C.), were dry-mixed in a 100 mL round-bottom flask contained a magnetic stir bar under argon. Heating the flask to for 24 hours led to a partially fluid suspension of stirring solids. The contents were heated an additional 15 hours at 150° C. before cooling. The solid mass is broken up and dissolve/suspended in 100 mL of water. The fine suspension of solids is suction filtered through a glass fiber pad on a #4 filter paper pad and freeze-dried to give 3.0 g of solid which is diluted with water to make a 0.067 g/mL solution. The resulting photoactivator exhibits a suitable excitation wavelength of about 380 nm and comprises about 9%, by weight of the photoactivator, of photoactive moiety.

### Poly(Vinyl Alcohol)-Naphthalene Carbamate Conjugate

Weighed out 1.38 g of poly(vinyl alcohol) (40% hydrolyzed; MW 72,000) in 25 mL flask with a magnetic stir bar. Added 14 mL of tetrahydrofuran (THF) to swell/suspend the polymer. Added 0.338 g of 1-naphthyl isocyanate in 1 mL of THF, placed under an argon atmosphere, covered with foil to shield from light, and stirred at room temperature for 4 days. The viscous slurry is transferred into 60 mL of methanol and stirred as 20 mL of aqueous 1.0 N sodium hydroxide is added. After 23 hours of stirring at room temperature the mixture is transferred into a 1 L flask, fitted with an air condenser and stirred at 60° C. under argon for 16 hours. The mixture is concentrated under reduced pressure (rotary evaporator) at 50° C., slurried again in 200 mL of water (solution pH 11) and concentrated again. The resulting residue is slurried in 150 mL of water and suction filtered to give a clear, yellowish solution. After freeze-drying 1.685 g of fluffy white powder is obtained and diluted with water to a 0.164 g/mL solution. The resulting photoactivator exhibits a suitable excitation wavelength of about 330 nm and comprises about 19%, by weight of the photoactivator, of photoactive moiety.

### Benzophenone-mPEG(2000) Carbamate Conjugate

4-Isocyanatobenzophenone (0.138 g) and poly(ethylene glycol) methyl ether (mPEG-2000, 1.500 g; Mₙ ca 2000, Tₘ=52° C.) were combined in a 10 mL flask with a magnetic stir bar under an argon atmosphere. The mixture is placed in a 80° C. oil bath and stirred as the mixture melted to provide a peach-colored slurry. After stirring for 17.5 hours the mixture is cooled to room temperature to form a solid mass which is partially dissolved in 140 mL of water. After suction filtering through a glass fiber pad on top of a #4 paper pad a clear aqueous solution is attained. Freeze-drying this solution led to 1.53 g of a fluffy white solid. This solid is diluted with water to provide a 0.0263 g/mL solution. The resulting photoactivator exhibits a suitable excitation wavelength of about 425 nm and comprises about 8%, by weight of the photoactivator, of photoactive moiety.

### Naphthalene-mPEG(500) Carbamate Conjugate

1-Naphthyl isocyanate (0.338 g) and poly(ethylene glycol) methyl ether (mPEG-550, 1.30 g; Mₙ ca 550, Tₘ=20° C.) were combined in 10 mL flask with a magnetic stir bar under an argon atmosphere, covered with foil to shield from light, and stirred at room temperature for 4 days. The mixture is diluted into 80 mL of water and stirred for 15 minutes. The cloudy solution is suction filtered through #3 filter paper to give a clear aqueous solution. This solution is freeze-dried to yield 1.65 g of a colorless oil which is diluted with water to provide a 0.0412 g/mL solution. The resulting photoactivator exhibits a suitable excitation wavelength of about 337 nm and comprises about 23%, by weight of the photoactivator, of photoactive moiety.

Poly(Acrylic Acid) Functionalized with Acridine Amide

Weighed out 0.427 g of 9-aminoacridine into a 25 mL 2-neck round-bottom flask with a magnetic stir bar and placed under an argon atmosphere. Added 10 mL of dioxane and stirred the resulting suspension overnight at room temperature under argon. Triethylamine (0.50 mL) is added to the 9-aminoacridine/dioxane suspension. A 25 mL 2-neck round-bottom flask with a thermocouple probe and a magnetic stir bar is charged with 4.00 g of poly(acryloyl chloride) solution (25% in dioxane, thus 1.00 g of polymer; polymer MW˜10,000) and placed under an argon atmosphere and cooled to 8° C. (thickened). The cold bath is removed and then 9-aminoacridine/dioxane slurry is added in one portion through a funnel and the mixture is left under an argon atmosphere. The mixture became thick with solids immediately; the temperature rose to 25° C. and subsided over 5 minutes. To aid stirring 5 mL more dioxane is added. The mixture is heated to 80° C. and stirring is continued under argon for 23 hours. The solids that were adhering to the sides of the flask were scraped and the entire contents were transferred to a 500 mL with the aid of 12.8 mL of 1.0 N sodium hydroxide solution and the mixture is magnetically stirred overnight. An additional 2.0 mL of 1.0 N sodium hydroxide is added to the pH 7-8 suspension of fine solids. An hour later an additional 2.0 mL of 1.0 N sodium hydroxide is added to the pH 9 suspension (less suspended solids now). After the resulting pH 11 mixture is stirred at room temperature for 3 days the pH dropped to 9-10. The sample is concentrated under reduced vacuum (rotary evaporator, 40° C.). After 50 mL of water is added to the residue it is concentrated again, and this step is repeated. The residue is suspended in 100 mL of water and suction filtered through #4 paper. The cloudy filtrate is diluted with water to 300 mL and filtered through #4 paper topped with a glass fiber pad to provide a clearer solution which is freeze-dried. The resulting 1.15 g of yellow, sticky, fibrous solid is diluted with water to provide a 0.0144 g/mL solution. The resulting photoactivator exhibits a suitable excitation wavelength of about 395 nm and comprises about 31%, by weight of the photoactivator, of photoactive moiety.

Poly(Acrylic Acid) Functionalized with Naphthalenemethyl Amide

A 25 mL 2-neck round-bottom flask with a magnetic stir bar is charged with 4.00 g of poly(acryloyl chloride) solution (25% in dioxane, thus 1.00 g of polymer; polymer MW˜10,000) and placed under an argon atmosphere. A solution of 0.346 g of 1-naphthylenemethylamine and 0.32 mL of triethylamine in 2 mL of tetrahydrofuran is added to the polymer/dioxane mixture over 5 minutes with stirring. The solution quickly forms a suspension of solids. After stirring for 24 hours at room temperature the reaction is transferred to a 100 mL flask and 19.8 mL of 1.0 M aqueous sodium hydroxide is added, the flask is capped (not under argon), and the cream-colored slurry is stirred for 16.5 hours at room temperature. After adding 2 mL of 1.0 N hydrochloric acid the mixture is concentrated under reduced pressure (rotary evaporator) at 40° C., suspended again in 50 mL of water and concentrated down to about 30 mL to give a suspension having a pH of 7-8. Subsequent addition of 1.0 mL of 1.0 N NaOH is followed by the drop wise addition of 1.0 N hydrochloric acid (approximately 1 mL) until the pH is between 9 and 10. An additional 30 mL of water is added and the mixture is concentrated under reduced pressure (rotary evaporator) at 50° C. to give 2.14 g of residue. This residue is partially dissolved/suspended in 100 mL of water and insolubles were removed by suction filtration. The resulting pH 7-8 solution is freeze-dried to give 1.78 g of an off-white, sticky, fibrous solid which is diluted with water to provide a 0.022 g/mL solution. The resulting photoactivator exhibits a suitable excitation wavelength of about 320 nm and comprises about 1%, by weight of the photoactivator, of photoactive moiety.

### Fluorescein-mPEG(550) Conjugate

Fluorescein 5-isothiocyanate (0.226 g) and poly(ethylene glycol) methyl ether (mPEG-550, 1.20 g; Mₙ ca 550, Tₘ=20° C.) were combined in 10 mL flask with a magnetic stir bar under an argon atmosphere. The mixture is placed in a 120° C. oil bath and stirred to provide an orange suspension. After 6 days at this temperature the mixture is nearly homogenous and is allowed to cool to room temperature. The residue is taken up in 100 mL of water, and after 18 hours the solution is centrifuged to remove undissolved materials. The supernatant is separated and the water is removed by freeze-drying to give 1.051 g of yellow oil which is taken up in water to provide a 0.0104 g/mL solution of the conjugate. The resulting photoactivator exhibits a suitable excitation wavelength of about 490 nm and comprises about 41%, by weight of the photoactivator, of photoactive moiety.

### Fluorescein-mPEG(2000) Conjugate

Fluorescein 5-isothiocyanate (0.226 g) and poly(ethylene glycol) methyl ether (mPEG-2000, 1.500 g; Mₙ ca 2000, Tₘ=52° C.) were combined in 10 mL flask with a magnetic stir bar under an argon atmosphere. The mixture is placed in a 100° C. oil bath and stirred as the mPEG-2000 melted to provide an orange suspension. After 3 days at this temperature the mixture is nearly homogenous and is allowed to cool to room temperature. The residue is taken up in 200 mL of water, and after 18 hours undissolved solids were removed by vacuum filtration. The water is removed by freeze-drying to give 1.514 g of yellow-orange solid which is taken up in water to provide a 0.014 g/mL solution of the conjugate. The resulting photoactivator exhibits a suitable excitation wavelength of about 460 nm and comprises about 12%, by weight of the photoactivator, of photoactive moiety.

### Gantrez-Aminoacridine Amide Conjugate

A 250 mL round-bottom flask is charged with 0.972 g of 9-aminoacridine and 23 ml THF. Stir under nitrogen while cooling in an ice water bath. Via a dry syringe transfer 1.5 ml of 2.5M butyllithium solution in hexanes to the flask. Remove the ice bath and continue to stir for 20 min at RT. Weigh out 2.925 g of Gantrez (anhydride form; M_{w} 216,000; Mₙ 80,000), and add 140 ml THF. Some material remained undissolved. Pour the mixture into the into the reaction flask at RT. Add 1 ml triethylamine. Heat to reflux. Continue to reflux for 35 days then cool to RT. An aqueous solution of 1.0 N sodium hydroxide (35 mL) is slowly added to reaction flask and the mixture is stirred another 16 hours at room temperature. The two-phase mixture is transferred to a 1 L flask with 100 mL of water and concentrated at 50° C. in vacuo using a rotary evaporator. Three additional cycles were performed adding 50 mL of water and concentrating to give 6.7 g of a tan/beige solid. This residue is taken up in 200 mL of water, suction filtered, and the filtrate is freeze-dried to provide 6.55 g of a light solid. An aliquot 1.0139 g of this sample is diluted with 20 ml of H₂O to give a 5 wt % aqueous solution.

### Phenothiazine-mPEG(2000) Carbamate Conjugate

To a 250 mL round-bottom flask containing a magnetic stir bar and fitted with a condenser connected to a Firestone valve (for vacuum and nitrogen introduction) is charged with 7.58 g of poly(ethylene glycol) methyl ether (mPEG-2000, Mₙ ca 2000, Tₘ=52° C.) and 90 mL of methylene chloride at RT. While stirring under nitrogen 1.0009 g Phenothiazine-10-carbonyl chloride is added at room temperature. The colorless solution changed to a pink color with some precipitation. To the mixture is added 0.0471 g of 4-(dimethylamino)pyridine and 0.58 mL of triethylamine. The mixture is heated to reflux for 96 hours. The mixture became darker and a slurry observed at the bottom of the flask. After cooling and adding an additional 50 mL of methylene chloride the mixture is extracted with 20 mL of 1M HCl and twice with 50 mL of water. The organic solution is dried over magnesium sulfate. After suction filtering the solvent is removed in vacuo at 46° C. using a rotary evaporator. The solid (9.10 g) residue is taken up in 400 mL of water to provide a milky white solution which is suction filtered through a combination of glass and paper fiber filter pads. Freeze-drying led to 7.16 g of a pure white solid. An aliquot of the solid 1.0085 g is dissolved in 10 ml H₂O to make a 10 wt. % aqueous solution.

### Gantrez-Naphthoic Hydrazide Diacylhydrazine Conjugate

A 250 mL round-bottom flask containing 4.20 g of Gantrez (anhydride form; M_{w} 216,000; Mₙ 80,000), 125 mL of tetrahydrofuran, and a magnetic stir bar is fitted with a condenser connected to a Firestone valve (for vacuum and nitrogen introduction) and stirred at RT under nitrogen. The polymer dissolved. The flask is then charged with 1-Naphthoic hydrazide 1.0019 g, and triethylamine 0.60 g at RT. Initially all the reagents were in solution, but became a purple color mixture over time. The mixture is heated to reflux for 48 hours, then cool to RT. An aqueous solution of 1.0 N sodium hydroxide (48.5 mL) is slowly added to reaction and the mixture is stirred another 16 hours at room temperature. The two-phase mixture is transferred to a 1 L flask with 100 mL of water and 50 ml THF and concentrated at 50° C. in vacuo using a rotary evaporator. Three additional cycles were performed adding 75 mL of water and concentrating to give 7.72 g of a tan/beige solid. This residue is taken up in 200 mL of water, suction filtered, and the filtrate is freeze-dried to provide 6.63 g of a light solid. An aliquot 1.07 g of this sample is diluted to a 10 wt % aqueous solution.

Each of the photoactivators exemplified above are found to be suitable photoactivators according to both the CHLORITE QUENCHING TEST METHOD and the INDIGO CARMINE BLEACHING TEST METHOD, as described hereinbefore.

### Consumer Product Examples

The following are non-limiting examples of various consumer product compositions of the present invention.

Automatic Dishwashing Cleaning Powder

| % (w/w) | 1 | 2 | 3 | 4* | 5* | 6 | 7 | 8 |
|---|---|---|---|---|---|---|---|---|
| PEG ester of | 0.05 | 0.1 | 0.5 | | 0.5 | 0.1 | 0.1 | 0.5 |
| thioxanthione- | | | | | | | | |
| 2-carboxylic acid ¹ | | | | | | | | |
| Eosin Y | | | | 0.05 | | | | |
| Sodium Chlorite | 30 | 15 | 5 | 30 | 20 | 5 | | |
| Sodium bromite | | | | | | | 15 | |
| Sodium chlorate | | | | | | | | 20 |
| Pentamine | 0.5 | | | | | | | 0.5 |
| cobalt | | | | | | | | |
| dichloride | | | | | | | | |
| Nanoparticle | | | 0.1 | | | 0.1 | | |
| Titanium | | | | | | | | |
| dioxide | | | | | | | | |
| Benzoquinone | | 0.1 | | | | | 1 | |
| Sodium tripoly | 10-30 | 10-30 | 10-30 | 10-30 | 10-30 | 10-30 | 10-30 | 10-30 |
| phosphate | | | | | | | | |
| SLF-18 poly- | 0.5-2 | 0.5-2 | 0.5-2 | 0.5-2 | 0.5-2 | 0.5-2 | 0.5-2 | 0.5-2 |
| tergent² | | | | | | | | |
| Alcosperse 246³ | 0-5 | 0-5 | 0-5 | 0-5 | 0-5 | 0-5 | 0-5 | 0-5 |
| Esterified | 0.1-6 | 0.1-6 | 0.1-6 | 0.1-6 | 0.1-6 | 0.1-6 | 0.1-6 | 0.1-6 |
| substituted | | | | | | | | |
| benzene | | | | | | | | |
| sulfonate⁴ | | | | | | | | |
| Polymer⁵ | 0.2-6 | 0.2-6 | 0.2-6 | 0.2-6 | 0.2-6 | 0.2-6 | 0.2-6 | 0.2-6 |
| Carbonate | 25-35 | 25-35 | 25-35 | 25-35 | 25-35 | 25-35 | 25-35 | 25-35 |
| 2.0 silicate | 7-15 | 7-15 | 7-15 | 7-15 | 7-15 | 7-15 | 7-15 | 7-15 |
| Enzyme | 0.5-3 | 0.5-3 | 0.5-3 | 0.5-3 | 0.5-3 | 0.5-3 | 0.5-3 | 0.5-3 |
| system⁶ | | | | | | | | |
| Sodium sulfate | 20-35 | 20-35 | 20-35 | 20-35 | 20-35 | 20-35 | 20-35 | 20-35 |
| Water, perfume | To | To | To | To | To | To | To | To |
| and other | 100% | 100% | 100% | 100% | 100% | 100% | 100% | 100% |
| components | | | | | | | | |

*In the examples denoted by an asterisk, the electron acceptor is diatomic oxygen which is present in aqueous solution upon dissolving the powder in water.
¹ Contains approximately 225 units of polyethylene glycol (“PEG”) moiety.
²Linear alcohol ethoxylate from Olin Corporation
³Sulfonated copolymer of acrylic acid from Alco Chemical Co.
⁴Such as those described above
⁵An anti-scaling polymer such as those described above
⁶One or more enzymes such as protease, mannaway, natalase, lipase, or mixtures thereof.

Automatic Dishwashing Cleaning Liquid/Gel

| % (w/w) | 1 | 2* | 3 | 4 | 5 | 6* | 7 | 8* | 9 | 10 | 11* |
|---|---|---|---|---|---|---|---|---|---|---|---|
| PEG ester of | 0.1 | 0.01 | | 0.05 | | 0.1 | 0.01 | | 0.05 | | 0.05 |
| thioxanthione- | | | | | | | | | | | |
| 2-carboxylic acid | | | | | | | | | | | |
| Eosin Y | | | 0.01 | | 0.05 | | | 0.01 | | 0.05 | |
| Sodium Chlorite | 5 | 20 | 20 | | | 5 | 20 | 20 | | | 10 |
| Sodium bromite | | | | 5 | | | | | 20 | | |
| Sodium chlorate | | | | | 15 | | | | | 10 | |
| Pentamine | | | 0.5 | | | | 0.05 | | | | |
| cobalt | | | | | | | | | | | |
| chloride | | | | | | | | | | | |
| AcceptorNano- | | | | 0.1 | 0.1 | | | | 0.1 | 0.1 | |
| particle | | | | | | | | | | | |
| titanium | | | | | | | | | | | |
| dioxide | | | | | | | | | | | |
| Benzoquinone | 1 | | | | | | | | | | |
| Sodium | | | | | | 10-25 | 10-25 | 10-25 | 10-25 | 10-25 | 10-25 |
| tripoly | | | | | | | | | | | |
| phosphate | | | | | | | | | | | |
| Polygel | | | | | | 1-2 | 1-2 | 1-2 | 1-2 | 1-2 | 1-2 |
| DKP¹ | | | | | | | | | | | |
| SLF-18 poly- | | | | | | 0-2 | 0-2 | 0-2 | 0-2 | 0-2 | 0-2 |
| tergent² | | | | | | | | | | | |
| Esterified | | | | | | 0.1-6 | 0.1-6 | 0.1-6 | 0.1-6 | 0.1-6 | 0.1-6 |
| substituted | | | | | | | | | | | |
| benzene | | | | | | | | | | | |
| sulfonate⁴ | | | | | | | | | | | |
| Polymer⁵ | | | | | | 0.2-6 | 0.2-6 | 0.2-6 | 0.2-6 | 0.2-6 | 0.2-6 |
| Hydrozincite | | | | | | 0-0.3 | 0-0.3 | 0-0.3 | 0-0.3 | 0-0.3 | 0-0.3 |
| Zinc sulfate | | | | | | 0-0.8 | 0-0.8 | 0-0.8 | 0-0.8 | 0-0.8 | 0-0.8 |
| Nitric acid | | | | | | 0.01-0.05 | 0.01-0.05 | 0.01-0.05 | 0.01-0.05 | 0.01-0.05 | 0.01-0.05 |
| (70%) | | | | | | | | | | | |
| Sulfuric acid | | | | | | 0-5 | 0-5 | 0-5 | 0-5 | 0-5 | 0-5 |
| NaOH | | | | | | 0-4 | 0-4 | 0-4 | 0-4 | 0-4 | 0-4 |
| KOH | | | | | | 0-15 | 0-15 | 0-15 | 0-15 | 0-15 | 0-15 |
| 2.0 silicate | | | | | | 0-20 | 0-20 | 0-20 | 0-20 | 0-20 | 0-20 |
| Sodium | | | | | | 0-8 | 0-8 | 0-8 | 0-8 | 0-8 | 0-8 |
| hypochloride | | | | | | | | | | | |
| Enzyme | | | | | | 0-1 | 0-1 | 0-1 | 0-1 | 0-1 | 0-1 |
| system⁶ | | | | | | | | | | | |
| 1,2- | | | | | | 0-1 | 0-1 | 0-1 | 0-1 | 0-1 | 0-1 |
| propanediol | | | | | | | | | | | |
| Boric acid | | | | | | 0-4 | 0-4 | 0-4 | 0-4 | 0-4 | 0-4 |
| Sodium | | | | | | 2-6 | 2-6 | 2-6 | 2-6 | 2-6 | 2-6 |
| perborate | | | | | | | | | | | |
| monohydrate | | | | | | | | | | | |
| Calcium | | | | | | 0-0.5 | 0-0.5 | 0-0.5 | 0-0.5 | 0-0.5 | 0-0.5 |
| chloride | | | | | | | | | | | |
| Sodium | | | | | | 0.1-6 | 0.1-6 | 0.1-6 | 0.1-6 | 0.1-6 | 0.1-6 |
| benzoate | | | | | | | | | | | |
| Water, | | | | | | Balance | Balance | Balance | Balance | Balance | Balance |
| perfume and | | | | | | to 100% | to 100% | to 100% | to 100% | to 100% | to 100% |
| other | | | | | | | | | | | |
| components | | | | | | | | | | | |

*In the examples denoted by an asterisk, the electron acceptor is diatomic oxygen which is present in aqueous solution upon dissolving the powder in water.
¹Polyacrylate thickener from ex 3V Co.
²Linear alcohol ethoxylate from Olin Corporation
3 Sulfonated copolymer of acrylic acid from Alco Chemical Co.
⁴Such as those described above
⁵An anti-scaling polymer such as those described above
⁶One or more enzymes such as protease, mannaway, natalase, lipase, or mixtures thereof.

Body Wash Compositions

| | 1 | 2 | 3* | 4 | 5* | 6 | 7* | 8 |
|---|---|---|---|---|---|---|---|---|
| Distilled Water | QS | QS | QS | QS | QS | QS | QS | QS |
| PEG ester of | 0.01 | 0.05 | | | 0.1 | 0.1 | 0.05 | |
| thioxanthione- | | | | | | | | |
| 2-carboxylic acid | | | | | | | | |
| (repeat units 225, | | | | | | | | |
| MW 10000 of PEG) | | | | | | | | |
| Eosin Y | | | 0.01 | 0.01 | | | | 0.01 |
| Sodium Chlorite | 1.0 | 0.5 | 1.0 | 0.5 | | | 0.5 | 1.0 |
| Sodium bromite | | | | | 0.5 | 1 | | |
| Pentamine cobalt | 0.5 | | | 0.5 | | | | |
| chloride | | | | | | | | |
| Nanoparticle | | 0.1 | | | | 0.1 | | |
| titanium | | | | | | | | |
| dioxide | | | | | | | | |
| Benzoquinone | | | | | | | | 1 |
| Acceptor | | | | | | | | |
| Sodium Tridecyl | | | | | | 10.54 | 10.54 | 10.54 |
| Ether Sulfate | | | | | | | | |
| Dehyton ML (Sodium | | | | | | 6.59 | 6.59 | 6.59 |
| Lauroamphoacetate) | | | | | | | | |
| (a) | | | | | | | | |
| Electrolyte ¹ | | | | | | 4.01 | 4.01 | 4.01 |
| 3-Ethoxylated | | | | | | 0.84 | 0.84 | 0.84 |
| Tridecyl Alcohol (b) | | | | | | | | |
| Cationic Polymer | | | | | | 0.35 | 0.35 | 0.35 |
| pH adjustment agent | | | | | | 0.23 | 0.23 | 0.23 |
| Aqupec Ser W300C ² | | | | | | 0.17 | 0.17 | 0.17 |
| Disodium EDTA | | | | | | 0.13 | 0.13 | 0.13 |
| Kathon CG | | | | | | 0.031 | 0.031 | 0.031 |
| Hydrogen peroxide | | | | | | 0.004 | 0.004 | 0.004 |
| solution, 20-40% | | | | | | | | |
| Soybean oil | | | | | | | 15 | |
| Petrolatum | | | | | | | | 13 |
| Glyceryl monooleate | | | | | | | | 2 |

*In the examples denoted by an asterisk, the electron acceptor is diatomic oxygen which is present in aqueous solution upon dissolving the powder in water.
¹ Suitable electrolytes are described in U.S. patent application Ser. No. 13/157,665.
² Acrylates/C10-30 Alkyl Acrylate Crosspolymer available from Presperse
(a) available as Dehyton ML from Cognis
(b) available as Iconol-TDA-3 or Lutensol-TDA-3 from BASF

Granular laundry detergent compositions for hand washing or top-loading washing machines

| % (w/w) | 1 | 2* | 3 | 4* | 5 | 6* | 7* | 8 |
|---|---|---|---|---|---|---|---|---|
| PEG ester of | 0.05 | 0.1 | 0.5 | | 0.5 | 0.1 | 0.1 | 0.5 |
| thioxanthione- | | | | | | | | |
| 2-carboxylic acid | | | | | | | | |
| Eosin y | | | | 0.05 | | | | |
| Sodium Chlorite | 30 | 15 | 5 | 30 | 20 | 5 | | |
| Sodium bromite | | | | | | | 20 | |
| Sodium | | | | | | | | 20 |
| chlorateAcceptor | | | | | | | | |
| Pentamine cobalt | 0.5 | | | | | | | 1 |
| chloride | | | | | | | | |
| Nanoparticle | | | 0.11 | | | | | |
| titanium | | | | | | | | |
| dioxide | | | | | | | | |
| benzoquinone | | | | | 1 | | | |
| Linear | 20 | 22 | 20 | 15 | 20 | 20 | 20 | 15 |
| alkylbenzene- | | | | | | | | |
| sulfonate | | | | | | | | |
| C₁₂₋₁₄ Dimethyl- | 0.7 | 0.2 | 1 | 0.6 | 0.0 | 0 | 1 | 0.6 |
| hydroxyethyl | | | | | | | | |
| ammonium chloride | | | | | | | | |
| AE3S (a) | 0.9 | 1 | 0.9 | 0.0 | 0.5 | 0.9 | 0.9 | 0.0 |
| AE7 (b) | 0.0 | 0.0 | 0.0 | 1 | 0.0 | 3 | 0.0 | 1 |
| Sodium | 5 | 0.0 | 4 | 9 | 2 | 0.0 | 4 | 9 |
| tripolyphosphate | | | | | | | | |
| Zeolite A | 0.0 | 1 | 0.0 | 1 | 4 | 1 | 0.0 | 1 |
| 1.6R Silicate | 7 | 5 | 2 | 3 | 3 | 5 | 2 | 3 |
| (SiO₂:Na₂O at | | | | | | | | |
| ratio 1.6:1) | | | | | | | | |
| Sodium carbonate | 25 | 20 | 25 | 17 | 18 | 19 | 25 | 17 |
| Polyacrylate | 1 | 0.6 | 1 | 1 | 1.5 | 1 | 1 | 1 |
| MW 4500 | | | | | | | | |
| Carboxymethyl | 1 | 0.3 | 1 | 1 | 1 | 1 | 1 | 1 |
| cellulose | | | | | | | | |
| Stainzyme ® | 0.1 | 0.2 | 0.1 | 0.2 | 0.1 | 0.1 | 0.1 | 0.2 |
| (20 mg active/g) | | | | | | | | |
| (c) | | | | | | | | |
| Protease | 0.1 | 0.1 | 0.1 | 0.1 | | 0.1 | 0.1 | 0.1 |
| (Savinase ®, | | | | | | | | |
| 32.89 mg active/g) | | | | | | | | |
| (d) | | | | | | | | |
| Amylase - Natalase ® | 0.1 | 0.0 | 0.1 | 0.0 | 0.1 | 0.1 | 0.1 | 0.0 |
| (8.65 mg active/g) | | | | | | | | |
| (e) | | | | | | | | |
| Lipase - Lipex ® | 0.03 | 0.07 | 0.3 | 0.1 | 0.07 | 0.4 | 0.3 | 0.1 |
| (18 mg active/g) | | | | | | | | |
| (f) | | | | | | | | |
| Fluorescent | 0.06 | 0.0 | 0.06 | 0.18 | 0.06 | 0.06 | 0.06 | 0.18 |
| Brightener 1 | | | | | | | | |
| Fluorescent | 0.1 | 0.06 | 0.1 | 0.0 | 0.1 | 0.1 | 0.1 | 0.0 |
| Brightener 2 | | | | | | | | |
| Diethylene- | 0.6 | 0.8 | 0.6 | 0.25 | 0.6 | 0.6 | 0.6 | 0.25 |
| triamine-penta- | | | | | | | | |
| acetic acid | | | | | | | | |
| MgSO₄ | 1 | 1 | 1 | 0.5 | 1 | 1 | 1 | 0.5 |
| Sulfate/Moisture | balance | balance | balance | balance | balance | balance | balance | balance |

*In the examples denoted by an asterisk, the electron acceptor is diatomic oxygen which is present in aqueous solution upon dissolving the powder in water.
(a) AE3S = sodium or ammonium salt of the sulfated oleochemically derived alcohol ethoxylates containing 2 or 3 ethylene oxide units
(b) AE7 = oleochemically derived alcohol ethoxylates containing an average of 7 ethylene oxide units
(c) Stainzyme = Trade name for amylase enzyme available from Novozymes
(d) Savinase = Trade name for protease enzyme available from Nonzymes
(e) Natalasae = Trade name for amylase enzyme available from Novozymes
(f) Lipex = Trade name for lipase enzyme available from Novozymes

Granular laundry detergent compositions for front-loading automatic washing machines.

| | 1 | 2 | 3 | 4 | 5* | 6 | 7* | 8* |
|---|---|---|---|---|---|---|---|---|
| PEG ester of | 0.05 | 0.1 | 0.5 | | 0.5 | 0.1 | 0.1 | 0.5 |
| thioxanthione- | | | | | | | | |
| 2-carboxylic acid | | | | | | | | |
| Eosin y | | | | 0.05 | | | | |
| Sodium Chlorite | 30 | 15 | 5 | 30 | 20 | 5 | | |
| Sodium bromite | | | | | | | 20 | |
| Acceptor | | | | | | | | 10 |
| Sodium chlorate | 8 | 7.1 | 7 | 6.5 | 7.5 | 7.5 | 6.5 | 7.5 |
| Pentamine cobalt | 0.5 | | | 1 | | | | |
| chloride | | | | | | | | |
| Nanoparticle | | 0.1 | | | | 0.5 | | |
| titanium dioxide | | | | | | | | |
| benzoquinone | | | 1 | | | | | |
| AE3S (a) | 0 | 4.8 | 0 | 5.2 | 4 | 4 | 5.2 | 4 |
| C12-14 Alkylsulfate | 1 | 0 | 1 | 0 | 0 | 0 | 0 | 0 |
| AE7 (b) | 2.2 | 0 | 3.2 | 0 | 0 | 0 | 0 | 0 |
| C₁₀₋₁₂ Dimethyl | 0.75 | 0.94 | 0.98 | 0.98 | 0 | 0 | 0.98 | 0 |
| hydroxyethyl- | | | | | | | | |
| ammonium | | | | | | | | |
| chloride | | | | | | | | |
| Crystalline layered | 4.1 | 0 | 4.8 | 0 | 0 | 0 | 0 | 0 |
| silicate | | | | | | | | |
| (δ-Na₂Si₂O₅) | | | | | | | | |
| Zeolite A | 5 | 0 | 5 | 0 | 2 | 2 | 0 | 2 |
| Citric Acid | 3 | 5 | 3 | 4 | 2.5 | 3 | 4 | 2.5 |
| Sodium Carbonate | 15 | 20 | 14 | 20 | 23 | 23 | 20 | 23 |
| Silicate 2R | 0.08 | 0 | 0.11 | 0 | 0 | 0 | 0 | 0 |
| (SiO₂:Na₂O at | | | | | | | | |
| ratio 2:1) | | | | | | | | |
| Soil release agent | 0.75 | 0.72 | 0.71 | 0.72 | 0 | 0 | 0.72 | 0 |
| Acrylic Acid/Maleic | 1.1 | 3.7 | 1.0 | 3.7 | 2.6 | 3.8 | 3.7 | 2.6 |
| Acid Copolymer | | | | | | | | |
| (CAS no. 29132-58-9) | | | | | | | | |
| Carboxymethyl- | 0.15 | 1.4 | 0.2 | 1.4 | 1 | 0.5 | 1.4 | 1 |
| cellulose | | | | | | | | |
| Protease - Purafect ® | 0.2 | 0.2 | 0.3 | 0.15 | 0.12 | 0.13 | 0.15 | 0.12 |
| (84 mg active/g) | | | | | | | | |
| Amylase Stainzyme | 0.2 | 0.15 | 0.2 | 0.3 | 0.15 | 0.15 | 0.3 | 0.15 |
| Plus ® (20 mg active/g) | | | | | | | | |
| Lipase - Lipex ® | 0.05 | 0.15 | 0.1 | 0 | 0 | 0 | 0 | 0 |
| (18.00 mg active/g) | | | | | | | | |
| Amylase - Natalase ® | 0.1 | 0.2 | 0 | 0 | 0.15 | 0.15 | 0 | 0.15 |
| (8.65 mg active/g) | | | | | | | | |
| Cellulase - Celluclean ™ | 0 | 0 | 0 | 0 | 0.1 | 0.1 | 0 | 0.1 |
| (15.6 mg active/g) | | | | | | | | |
| Na salt of Ethylenediamine- | 0.2 | 0.2 | 0.2 | 0.2 | 0.2 | 0.2 | 0.2 | 0.2 |
| N,N′-disuccinic acid, | | | | | | | | |
| (S,S) isomer (EDDS) | | | | | | | | |
| Hydroxyethane di- | 0.2 | 0.2 | 0.2 | 0.2 | 0.2 | 0.2 | 0.2 | 0.2 |
| phosphonate (HEDP) | | | | | | | | |
| MgSO₄ | 0.42 | 0.42 | 0.42 | 0.42 | 0.4 | 0.4 | 0.42 | 0.4 |
| Perfume | 0.5 | 0.6 | 0.5 | 0.6 | 0.6 | 0.6 | 0.6 | 0.6 |
| Suds suppressor | 0.05 | 0.1 | 0.05 | 0.1 | 0.06 | 0.05 | 0.1 | 0.06 |
| agglomerate | | | | | | | | |
| Soap | 0.45 | 0.45 | 0.45 | 0.45 | 0 | 0 | 0.45 | 0 |
| Sulfate/Water & | balance | balance | balance | balance | balance | balance | balance | balance |
| Miscellaneous | | | | | | | | |

*In the examples denoted by an asterisk, the electron acceptor is diatomic oxygen which is present in aqueous solution upon dissolving the powder in water.
(a) AE3S = sodium or ammonium salt of the sulfated oleochemically derived alcohol ethoxylates containing 2 or 3 ethylene oxide units
(b) AE7 = oleochemically derived alcohol ethoxylates containing an average of 7 ethylene oxide units
Purafect = Trade name for protease enzyme from Genencor
Celluclean = Trade name for cellulose enzyme from Novozymes
Stainzyme = Trade name for amylase enzyme available from Novozymes
Natalasae = Trade name for amylase enzyme available from Novozymes
Lipex = Trade name for lipase enzyme available from Novozymes

Any of the above Granular laundry detergent compositions are used to launder fabrics at a concentration of 7000 to 10000 ppm in water, 20-90° C., and a 5:1 water:cloth ratio. The typical pH is about 10. The fabrics are then dried. In one aspect, the fabrics are actively dried using a dryer. In one aspect, the fabrics are actively dried using an iron. In another aspect, the fabrics are merely allowed to dry on a line wherein they are exposed to air and optionally sunlight.

Heavy Duty Liquid laundry detergent compositions

| | 1 | 2* | 3 | 4 | 5* | 6 | 7* | 8* | 9* | 10 | 11* | 12 | 13* | 14* |
|---|---|---|---|---|---|---|---|---|---|---|---|---|---|---|
| PEG ester of | 0.1 | 0.01 | | 0.05 | | 0.1 | 0.5 | 0.5 | 0.1 | 0.1 | 0.5 | | 0.1 | |
| thioxanthione- | | | | | | | | | | | | | | |
| 2-carboxylic acid | | | | | | | | | | | | | | |
| Eosin y | | | 0.01 | | 0.05 | | | | | | | 0.5 | | 0.1 |
| Sodium Chlorite | 5 | 20 | 20 | | | 15 | 5 | 20 | 5 | | | 15 | 5 | 5 |
| Sodium bromite | | | | 20 | | | | | | 5 | | | | |
| Sodium | | | | | | | | | | | 20 | | | |
| chlorateAcceptor | | | | | | | | | | | | | | |
| Pentamine | 0.5 | | | 1 | | | | | | | | | | |
| cobalt | | | | | | | | | | | | | | |
| chloride | | | | | | | | | | | | | | |
| Nanoparticle | | | | | | 0.1 | | | | | | 0.5 | | |
| Titanium | | | | | | | | | | | | | | |
| dioxide | | | | | | | | | | | | | | |
| Benzoquinone | | | 0.1 | | | | | | | 5 | | | | |
| AES C₁₂₋₁₅ | 0 | 0 | 0 | 0 | 0 | 6.32 | 0 | 0 | 6.32 | 0 | 0 | 6.32 | 0 | 0 |
| alkyl ethoxy | | | | | | | | | | | | | | |
| (1.8) sulfate | | | | | | | | | | | | | | |
| AE3S (a) | 0 | 0 | 0 | 0 | 0 | 0 | 2.4 | 0 | 0 | 2.4 | 0 | 0 | 2.4 | 0 |
| Linear alkyl | 0 | 0 | 0 | 0 | 0 | 3.3 | 5 | 8 | 3.3 | 5 | 8 | 3.3 | 5 | 8 |
| benzene | | | | | | | | | | | | | | |
| sulfonate | | | | | | | | | | | | | | |
| Sodium | 0 | 0 | 0 | 0 | 0 | 0.04 | 1.6 | 1.2 | 0.04 | 1.6 | 1.2 | 0.04 | 1.6 | 1.2 |
| formate | | | | | | | | | | | | | | |
| Sodium | 0 | 0 | 0 | 0 | 0 | 1.9 | 1.7 | 2.5 | 1.9 | 1.7 | 2.5 | 1.9 | 1.7 | 2.5 |
| hydroxide | | | | | | | | | | | | | | |
| Monoethanol | 0 | 0 | 0 | 0 | 0 | 0.7 | 0 | 0 | 0.7 | 0 | 0 | 0.7 | 0 | 0 |
| amine | | | | | | | | | | | | | | |
| Diethylene | 0 | 0 | 0 | 0 | 0 | 0.0 | 0 | 0 | 0.0 | 0 | 0 | 0.0 | 0 | 0 |
| glycol | | | | | | | | | | | | | | |
| AE9 (b) | 0 | 0 | 0 | 0 | 0 | 0.3 | 0 | 0 | 0.3 | 0 | 0 | 0.3 | 0 | 0 |
| AE7 (c) | 0 | 0 | 0 | 0 | 0 | 0 | 2.4 | 6 | 0 | 2.4 | 6 | 0 | 2.4 | 6 |
| Ethylene | 0 | 0 | 0 | 0 | 0 | 0.07 | 0.5 | 0.11 | 0.07 | 0.5 | 0.11 | 0.07 | 0.5 | 0.11 |
| Diamine | | | | | | | | | | | | | | |
| Disuccinic | | | | | | | | | | | | | | |
| acid | | | | | | | | | | | | | | |
| Citric Acid | 0 | 0 | 0 | 0 | 0 | 1.98 | 0.9 | 2.5 | 1.98 | 0.9 | 2.5 | 1.98 | 0.9 | 2.5 |
| C₁₂₋₁₄ | 0 | 0 | 0 | 0 | 0 | 0.37 | 0 | 0 | 0.37 | 0 | 0 | 0.37 | 0 | 0 |
| dimethyl | | | | | | | | | | | | | | |
| Amine Oxide | | | | | | | | | | | | | | |
| C₁₂₋₁₈ Fatty | 0 | 0 | 0 | 0 | 0 | 0.99 | 1.2 | 0 | 0.99 | 1.2 | 0 | 0.99 | 1.2 | 0 |
| Acid | | | | | | | | | | | | | | |
| 4-formyl- | 0 | 0 | 0 | 0 | 0 | 0 | 0.05 | 0.02 | 0 | 0.05 | 0.02 | 0 | 0.05 | 0.02 |
| phenylboronic | | | | | | | | | | | | | | |
| acid | | | | | | | | | | | | | | |
| Borax | 0 | 0 | 0 | 0 | 0 | 0.75 | 0 | 1.07 | 0.75 | 0 | 1.07 | 0.75 | 0 | 1.07 |
| Ethanol | 0 | 0 | 0 | 0 | 0 | 0.89 | 0 | 3 | 0.89 | 0 | 3 | 0.89 | 0 | 3 |
| Ethoxylated | 0 | 0 | 0 | 0 | 0 | 0.17 | 0.0 | 0.0 | 0.17 | 0.0 | 0.0 | 0.17 | 0.0 | 0.0 |
| (EO₁₅) | | | | | | | | | | | | | | |
| tetraethylene | | | | | | | | | | | | | | |
| pentamine | | | | | | | | | | | | | | |
| Ethoxylated | 0 | 0 | 0 | 0 | 0 | 0.4 | 1 | 1 | 0.4 | 1 | 1 | 0.4 | 1 | 1 |
| hexamethylene | | | | | | | | | | | | | | |
| diamine | | | | | | | | | | | | | | |
| 1,2- | 0 | 0 | 0 | 0 | 0 | 3.3 | 0.5 | 2 | 3.3 | 0.5 | 2 | 3.3 | 0.5 | 2 |
| Propanediol | | | | | | | | | | | | | | |
| Protease (40.6 | 0 | 0 | 0 | 0 | 0 | 0.9 | 0.7 | 0.6 | 0.9 | 0.7 | 0.6 | 0.9 | 0.7 | 0.6 |
| mg active/g) | | | | | | | | | | | | | | |
| Mannanase: | 0 | 0 | 0 | 0 | 0 | 0.06 | 0.04 | 0.045 | 0.06 | 0.04 | 0.045 | 0.06 | 0.04 | 0.045 |
| Mannaway ® | | | | | | | | | | | | | | |
| (25 mg | | | | | | | | | | | | | | |
| active/g) | | | | | | | | | | | | | | |
| Amylase: | 0 | 0 | 0 | 0 | 0 | 0.1 | 0 | 0.4 | 0.1 | 0 | 0.4 | 0.1 | 0 | 0.4 |
| Stainzyme ® | | | | | | | | | | | | | | |
| (15 mg | | | | | | | | | | | | | | |
| active/g) | | | | | | | | | | | | | | |
| Amylase: | 0 | 0 | 0 | 0 | 0 | 0.15 | 0.07 | 0 | 0.15 | 0.07 | 0 | 0.15 | 0.07 | 0 |
| Natalase ® | | | | | | | | | | | | | | |
| (29 mg | | | | | | | | | | | | | | |
| active/g) | | | | | | | | | | | | | | |
| Lipex ® (18 | 0 | 0 | 0 | 0 | 0 | 0.1 | 0.2 | 0 | 0.1 | 0.2 | 0 | 0.1 | 0.2 | 0 |
| mg active/g) | | | | | | | | | | | | | | |
| Water, | Bal | Bal | Bal | Bal | Bal | Bal | Bal | Bal | Bal | Bal | Bal | Bal | Bal | Bal |
| perfume, dyes | | | | | | | | | | | | | | |
| & other | | | | | | | | | | | | | | |
| components | | | | | | | | | | | | | | |

*In the examples denoted by an asterisk, the electron acceptor is diatomic oxygen which is present in aqueous solution upon dissolving the powder in water.
(a) AE3S = sodium or ammonium salt of the sulfated oleochemically derived alcohol ethoxylates containing 2 or 3 ethylene oxide units
(c) AE7 = oleochemically derived alcohol ethoxylates containing an average of 7 ethylene oxide units
(b) AE9 = oleochemically derived alcohol ethoxylates containing an average of 9 ethylene oxide units
HSAS = Sodium alkyl sulfate (acid form)
Mannaway = Trade name for Mannanase enzyme available from Novozymes Protease
Stainzyme = Trade name for amylase enzyme available from Novozymes
Natalasae = Trade name for amylase enzyme available from Novozymes
Lipex = Trade name for lipase enzyme available from Novozymes

### Unit Dose Laundry Detergent Compositions

It will be understood that such unit dose formulations can comprise one or multiple compartments. The following unit dose laundry detergent formulations of the present invention are provided below.

| Ingredients | 1 | 2 | 3* | 4* | 5* | 6 | 7 |
|---|---|---|---|---|---|---|---|
| PEG ester of | 0.01 | 0.05 | 0.02 | 0.1 | 0.05 | | |
| thioxanthione- | | | | | | | |
| 2-carboxylic acid | | | | | | | |
| Eosin y | | | | | | 0.05 | 0.05 |
| Sodium Chlorite | 5 | 1 | 3 | 3 | | | 3 |
| Sodium bromite | | | | | 5 | | |
| Sodium | | | | | | 3 | |
| chlorateAcceptor | | | | | | | |
| Pentamine cobalt | 1 | | | | | | 0.5 |
| chloride | | | | | | | |
| Nanoparticle | | 0.1 | | | | | |
| titanium dioxide | | | | | | | |
| benzoquinone | | | | | | 1 | |
| Alkylbenzene | 14.5 | 14.5 | 14.5 | 14.5 | 14.5 | 14.5 | 14.5 |
| sulfonic acid | | | | | | | |
| C 11-13, 23.5% | | | | | | | |
| 2-phenyl isomer | | | | | | | |
| C₁₂₋₁₄ alkyl | 7.5 | 7.5 | 7.5 | 7.5 | 7.5 | 7.5 | 7.5 |
| ethoxy 3 | | | | | | | |
| sulfate | | | | | | | |
| C₁₂₋₁₄ alkyl | 13.0 | 13.0 | 13.0 | 13.0 | 13.0 | 13.0 | 13.0 |
| 7-ethoxylate | | | | | | | |
| Citric Acid | 0.6 | 0.6 | 0.6 | 0.6 | 0.6 | 0.6 | 0.6 |
| Fatty Acid | 14.8 | 14.8 | 14.8 | 14.8 | 14.8 | 14.8 | 14.8 |
| Enzymes (as % | 1.7 | 1.7 | 1.7 | 1.7 | 1.7 | 1.7 | 1.7 |
| raw material | | | | | | | |
| not active) | | | | | | | |
| Protease (as % | 0.05 | 0.1 | 0.02 | 0.03 | 0.03 | 0.1 | 0.02 |
| active) | | | | | | | |
| Ethoxylated | 4.0 | 4.0 | 4.0 | 4.0 | 4.0 | 4.0 | 4.0 |
| Polyethylenimine¹ | | | | | | | |
| Hydroxyethane | 1.2 | 1.2 | 1.2 | 1.2 | 1.2 | 1.2 | 1.2 |
| diphosphonic acid | | | | | | | |
| Brightener | 0.3 | 0.3 | 0.3 | 0.3 | 0.3 | 0.3 | 0.3 |
| P-diol | 15.8 | 13.8 | 13.8 | 13.8 | 13.8 | 13.8 | 13.8 |
| Glycerol | 6.1 | 6.1 | 6.1 | 6.1 | 6.1 | 6.1 | 6.1 |
| Monoethanol amine | 8.0 | 8.0 | 8.0 | 8.0 | 8.0 | 8.0 | 8.0 |
| TIPA | — | — | 2.0 | — | — | — | 2.0 |
| Triethanol amine | — | 2.0 | — | — | — | 2.0 | — |
| Cumene sulphonate | — | — | — | — | 2.0 | — | — |
| cyclohexyl | | | | 2.0 | | | |
| dimethanol | | | | | | | |
| Water | 10 | 10 | 10 | 10 | 10 | 10 | 10 |
| Structurant | 0.14 | 0.14 | 0.14 | 0.14 | 0.14 | 0.14 | 0.14 |
| Perfume | 1.9 | 1.9 | 1.9 | 1.9 | 1.9 | 1.9 | 1.9 |
| Buffers | To | To | To | To | To | To | To |
| (monoethanolamine) | pH 8.0 | pH 8.0 | pH 8.0 | pH 8.0 | pH 8.0 | pH 8.0 | pH 8.0 |
| Solvents (1,2 | To | To | To | To | To | To | To |
| propanediol, | 100% | 100% | 100% | 100% | 100% | 100% | 100% |
| ethanol) | | | | | | | |

*In the examples denoted by an asterisk, the electron acceptor is diatomic oxygen which is present in aqueous solution upon dissolving the powder in water.
¹Polyethylenimine (MW = 600) with 20 ethoxylate groups per —NH.
TIPA
*Other optional agents/components include suds suppressors, structuring agents such as those based on Hydrogenated Castor Oil (preferably Hydrogenated Castor Oil, Anionic Premix), solvents and/or Mica pearlescent aesthetic enhancer.

Hand Dish and Hard Surface Cleaner Compositions

| Examples (% w/w) | 1* | 2* | 3 | 4 | 5* | 6 | 7* | 8* | 9* | 10 | 11* | 12 | 13* | 14* | 15 |
|---|---|---|---|---|---|---|---|---|---|---|---|---|---|---|---|
| PEG ester of | 0.1 | 0.01 | | | 0.05 | | 0.1 | 0.01 | | | 0.05 | | 0.1 | 0.01 | |
| thioxanthione- | | | | | | | | | | | | | | | |
| 2-carboxylic | | | | | | | | | | | | | | | |
| acid | | | | | | | | | | | | | | | |
| Eosin y | | | 0.1 | 0.01 | | 0.05 | | | 0.1 | 0.01 | | 0.05 | | | 0.1 |
| Sodium Chlorite | 5 | 20 | 5 | 20 | | | 5 | 20 | 5 | 10 | | | 5 | 10 | 5 |
| Sodium bromite | | | | | | 20 | | | | | 5 | | | | |
| Sodium | | | | | 5 | | | | | | | 15 | | | |
| chlorateAcceptor | | | | | | | | | | | | | | | |
| Pentamine | | | 1 | | | 0.5 | | | | | | | | | |
| cobalt | | | | | | | | | | | | | | | |
| chloride | | | | | | | | | | | | | | | |
| Nanoparticle | | | | 1 | | | | | | 5 | | | | | |
| titanium | | | | | | | | | | | | | | | |
| dioxide | | | | | | | | | | | | | | | |
| Benzoquinone | | | | | | | | | | | | 0.5 | | | 1 |
| Alkyl | | | | | | | 16 | 28.0 | 25.0 | 27.0 | 20.0 | 13 | 17 | 29 | 20 |
| ethoxy | | | | | | | | | | | | | | | |
| sulfate | | | | | | | | | | | | | | | |
| AEₓS** | | | | | | | | | | | | | | | |
| Amine | | | | | | | 5.0 | 7.0 | 7.0 | 5.0 | 5.0 | 4.5 | 6.0 | 7.0 | 6.5 |
| oxide | | | | | | | | | | | | | | | |
| C₉₋₁₁ EO₈ | | | | | | | 5 | — | — | 3.0 | 5.0 | — | — | — | 6.5 |
| Ethylan | | | | | | | — | — | 3.0 | — | — | — | — | — | — |
| 1008 ® | | | | | | | | | | | | | | | |
| Lutensol ® | | | | | | | — | — | — | — | 5.0 | 4 | 5 | — | — |
| TO 7 | | | | | | | | | | | | | | | |
| GLDA¹ | | | | | | | 0.7 | — | — | — | 1.0 | 0.7 | 0.7 | — | 1.0 |
| DTPMP² | | | | | | | — | — | — | — | 0.5 | — | — | — | — |
| DTPA³ | | | | | | | | — | 1.0 | — | — | | | | |
| MGDA⁴ | | | | | | | | — | — | 1.0 | — | | | | |
| Sodium | | | | | | | — | — | 1.0 | — | 0.5 | — | 0.2 | — | — |
| citrate | | | | | | | | | | | | | | | |
| Solvent | | 2 | 3 | | | | 1.3 | 2.5 | 4.0 | 3.0 | 2.0 | 2.0 | 2.0 | 4.0 | 2.0 |
| Polypropylene | | | | | | | 0.5 | 1.0 | 0.5 | 1.0 | | 0.5 | 0.5 | 1.0 | 0.4 |
| glycol | | | | | | | | | | | | | | | |
| (Mₙ = 2000) | | | | | | | | | | | | | | | |
| Sodium | | | | | | | 0.8 | 0.5 | 1.0 | 1.0 | 0.5 | 0.5 | 0.4 | 1.5 | 0.5 |
| chloride | | | | | | | | | | | | | | | |
| Water | | | | | | | to | to | to | to | to | to | to | to | to |
| | | | | | | | balance | balance | balance | balance | balance | balance | balance | balance | balance |

*In the examples denoted by an asterisk, the electron acceptor is diatomic oxygen which is present in aqueous solution upon dissolving the powder in water.
**Number of carbon atoms in the alkyl chain is between 12 and 13; and x is between 0.5 and 2.
Ethylan 1008 ® is a nonionic surfactant based on a synthetic primary alcohol, commercially available from AkzoNobel.
Lutensol ® TO 7 is nonionic surfactant made from a saturated iso-C₁₃ alcohol.
Solvent is ethanol.
Amine oxide is coconut dimethyl amine oxide.
¹Glutamic-N,N-diacetic acid
²Diethylenetriamine penta methylphosphonic acid
**Examples may have other optional ingredients such as dyes, opacifiers, perfumes, preservatives, hydrotropes, processing aids, salts, stabilizers, etc.
*Number of carbon atoms in the alkyl chain is between 12 and 13; and x is between 0.5 and 2.
Ethylan 1008 ® is a nonionic surfactant based on a synthetic primary alcohol, commercially available from Akzo Nobel.
Lutensol ® TO 7 is nonionic surfactant made from a saturated iso-C₁₃ alcohol.
Solvent is ethanol.
Amine oxide is coconut dimethyl amine oxide.
¹Glutamic-N,N-diacetic acid
²Diethylenetriamine penta methylphosphonic acid
³Diethylene triamine pentaacetic acid
⁴Methyl glycine diacetic acid
*** Examples may have other optional ingredients such as dyes, opacifiers, perfumes, preservatives, hydrotropes, processing aids, salts, stabilizers, etc.

It should be understood that every maximum numerical limitation given throughout this specification would include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A consumer product composition comprising:
(a) a water soluble organic photoactivator;
(b) an electron acceptor which accepts an electron from the photoactivator when the photoactivator is in a photo-excited state and/or reduced state; and
(c) a benefit active precursor which converts into a benefit active agent via electron transfer.

2. The consumer product composition of claim 1, wherein the electron acceptor is not covalently linked to the photoactivator.

3. The consumer product composition of claim 1, wherein the electron acceptor is selected from the group consisting of organic species containing nitrogen, organic species containing sulfur, organic species containing oxygen, organic species containing phosphorus, anions of inorganic salts, and mixtures thereof.

4. The consumer product composition of claim 1, wherein the electron acceptor is selected from the group consisting of viologens, 2,2′ bipyridinium, para-Benzoquinone, 2,3-Dichloro-5,6-dicyano-p-benzoquinone, Tetrahydroxy-1,4-quinone hydrate, 2,5-di-tert-butylhydroquinone, tert-Butylhydroquinone, Anthraquinone, Diaminoanthroquinone, Anthraquinone-2-sulfonic acid, Anthracene, Dicyanobenzene, Chloropentaamine cobalt dichloride, Silver nitrate, Iron Sulfate, Titanium Dioxide, Zinc Oxide, Cadmium Selenide, Thiamine hydrochloride, Thiamine pyrophosphate, Ammonium persulfate, Sodium persulfate, Potassium persulfate, (2,2,6,6-Tetramethylpiperidin-1-yl)oxy, Dimethylthiourea, Tetranitromethane, Lithium acetoacetate, Oxaloacetic acid, Sodium ascorbate, 2,6-Dicholorophenolindophenol, 4-methoxyphenol, 4-Methylmorpholine N-oxide, 4-tert-Butylcatechol, Allopurinol, Pyridoxal 5′-phosphate, pyridoxal hydrochloride, Sodium benzoate, Sodium Nitrate, Sodium Nitrite, Diatomic Oxygen, and mixtures thereof.

5. The consumer product composition of claim 1, wherein the benefit active precursor has the formula:
A[XOₙ]ₘ
wherein
A is selected from the group consisting of monovalent cations, divalent cations, and trivalent cations; preferably A is selected from the group consisting of Aluminum, Barium, Calcium, Cobalt, Chromium, Copper, Iron, Lithium, Potassium, Rubidium, Magnesium, Manganese, Molybdenum, Nickel, Sodium, Titanium, Vanadium, Zinc, ammonium, alkyl-ammonium, aryl-ammonium, and mixtures thereof; more preferably A is selected from the group consisting of lithium, sodium, potassium, magnesium, calcium, ammonium, and mixtures thereof;
X is selected from the group consisting of chlorine, bromine, iodine, and mixtures thereof;
n is 1, 2, 3, or 4, preferably n is 2, 3, or 4; and
m is 1, 2, or 3.

6. A consumer product composition, wherein the consumer product composition is a solid composition comprising:
(a) a water soluble organic photoactivator; and
(b) a benefit active precursor having the formula:
A[XOₙ]ₘ
wherein
A is selected from the group consisting of monovalent cations, divalent cations, and trivalent cations; preferably A is selected from the group consisting of Aluminum, Barium, Calcium, Cobalt, Chromium, Copper, Iron, Lithium, Potassium, Rubidium, Magnesium, Manganese, Molybdenum, Nickel, Sodium, Titanium, Vanadium, Zinc, ammonium, alkyl-ammonium, aryl-ammonium, and mixtures thereof; more preferably A is selected from the group consisting of lithium, sodium, potassium, magnesium, calcium, ammonium, and mixtures thereof;
X is selected from the group consisting of chlorine, bromine, iodine, and mixtures thereof;
n is 1, 2, 3, or 4, preferably n is 2, 3, or 4; and
m is 1, 2, or 3.

7. The consumer product composition of claim 6, wherein the consumer product composition is water soluble.

8. The consumer product composition of claim 7, wherein the consumer product composition is dissolved in water to form an aqueous solution comprising an electron acceptor which accepts an electron from the photoactivator when the photoactivator is in a photo-excited state and/or reduced state, wherein the electron acceptor is diatomic oxygen.

9. The consumer product composition of claim 1, wherein the photoactivator comprises less than about 35%, by weight of photoactivator, of a photoactive moiety.

10. The consumer product composition of claim 1, wherein the photoactivator comprises less than about 2%, by weight of photoactivator, of a photoactive moiety.

11. The consumer product composition of claim 1, wherein the photoactivator can be activated to the photo-excited state by excitation with incident radiation of a wavelength between about 350 nm and about 750 nm.

12. The consumer product composition of claim 1, wherein the photoactivator can be activated to the photo-excited state by excitation with incident radiation of a wavelength between about 350 nm and about 420 nm.

13. The consumer product composition of claim 1, wherein the photo-excited state of the photoactivator has an energy greater than about 100 kJ/mol more than a ground state of the photoactivator.

14. The consumer product composition of claim 1, wherein the water soluble organic photoactivator comprises a photoactive moiety is selected from the group consisting of 1,1′-biphenyl-4,4′-diamine, 1,1′-biphenyl-4-amine, benzophenone, 1,1′-biphenyl-4,4′-diol, 1,1′-biphenyl-4-amine, 1,1′-biphenyl-4-ol, 1,1′:2′,1″-terphenyl, 1,1′:3′,1″-terphenyl, 1,1′:4′,1″:4″,1′″-quaterphenyl, 1,1′:4′,1″-terphenyl, 1,10-phenanthroline, 1,1′-biphenyl, 1,2,3,4-dibenzanthracene, 1,2-benzenedicarbonitrile, 1,3-isobenzofurandione, 1,4-naphthoquinone, 1,5-naphthalenediol, 10H-phenothiazine, 10H-phenoxazine, 10-methylacridone, 1-acetonaphthone, 1-chloroanthraquinone, 1-hydroxyanthraquinone, 1-naphthalenecarbonitrile, 1-naphthalenecarboxaldehyde, 1-naphthalenesulfonic acid, 1-naphthalenol, 2(1H)-quinolinone, 2,2′-biquinoline, 2,3-naphthalenediol, 2,6-dichlorobenzaldehyde, 21H,23H-porphine, 2-aminoanthraquinone, 2-benzoylthiophene, 2-chlorobenzaldehyde, 2-chlorothioxanthone, 2-ethylanthraquinone, 2H-1-benzopyran-2-one, 2-methoxythioxanthone, 2-methyl-1,4-naphthoquinone, 2-methyl-9(10-methyl)-acridinone, 2-methylanthraquinone, 2-methylbenzophenone, 2-naphthalenamine, 2-naphthalenecarboxylic acid, 2-naphthalenol, 2-nitro-9(10-methyl)-acridinone, 9(10-ethyl)-acridinone, 3,6-qcridinediamine, 3,9-dibromoperylene, 3,9-dicyanophenanthrene, 3-benzoylcoumarin, 3-methoxy-9-cyanophenanthrene, 3-methoxythioxanthone, 3′-methylacetophenone, 4,4′-dichlorobenzophenone, 4,4′-dimethoxybenzophenone, 4-bromobenzophenone, 4-chlorobenzophenone, 4′-fluoroacetophenone, 4-methoxybenzophenone, 4′-methylacetophenone, 4-methylbenzaldehyde, 4-methylbenzophenone, 4-phenylbenzophenone, 6-methylchromanone, 7-(diethylamino)coumarin, 7H-benz[de]anthracen-7-one, 7H-benzo[c]xanthen-7-one, 7H-furo[3,2-g][1]benzopyran-7-one, 9(10H)-acridinone, 9(10H)-anthracenone, 9(10-methyl)-acridinone, 9(10-phenyl)-acridinon, 9,10-anthracenedione, 9-acridinamine, 9-cyanophenanthrene, 9-fluorenone, 9H-carbazole, 9H-fluoren-2-amine, 9H-fluorene, 9H-thioxanthen-9-ol, 9H-thioxanthen-9-one, 9H-thioxanthene-2,9-diol, 9H-xanthen-9-one, acetophenone, acridene, acridine, acridone, anthracene, anthraquinone, anthrone, α-tetralone, benz[a]anthracene, benzaldehyde, benzamide, benzo[a]coronene, benzo[a]pyrene, benzo[f]quinoline, benzo[ghi]perylene, benzo[rst]pentaphene, benzophenone, benzoquinone, 2,3,5,6-tetramethyl, chrysene, coronene, dibenz[a,h]anthracene, dibenzo[b,def]chrysene, dibenzo[c,g]phenanthrene, dibenzo[def,mno]chrysene, dibenzo[def,p]chrysene, DL-tryptophan, fluoranthene, fluoren-9-one, fluorenone, isoquinoline, methoxycoumarin, methylacridone, michler's ketone, naphthacene, naphtho[1,2-g]chrysene, N-methylacridone, p-benzoquinone, p-benzoquinone, 2,3,5,6-tetrachloro, pentacene, phenanthrene, phenanthrenequinone, phenanthridine, phenanthro[3,4-c]phenanthrene, phenazine, phenothiazine, p-methoxyacetophenone, pyranthrene, pyrene, quinoline, quinoxaline, riboflavin 5′-(dihydrogen phosphate), thioxanthone, thymidine, xanthen-9-one, xanthone, and mixtures thereof.

15. The consumer product composition of claim 1, wherein the water soluble organic photoactivator comprises a photoactive moiety selected from the group consisting of xanthone, xanthene, thioxanthone, thioxanthene, phenothiazine, fluorescein, benzophenone, alloxazine, isoalloxazine, flavin, and mixtures thereof.

16. The consumer product composition of claim 1, wherein the photoactive moiety is thioxanthone.

17. The consumer product composition of claim 1, wherein the water soluble organic photoactivator comprises a hydrophilic moiety selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphoinic acid, ethylene imine, and mixtures thereof.

18. The consumer product composition of claim 1, further comprising an adjunct ingredient selected from the group consisting of nonionic surfactants, cationic surfactants, zwitterionic surfactants, amphoteric surfactants, builders, structurants, thickeners, clay soil removal agents, anti-redeposition agents, polymeric soil release agents, polymeric dispersing agents, polymeric grease consumer product agents, enzymes, enzyme stabilizing systems, bleaching compounds, bleaching agents, bleach activators, bleach catalysts, brighteners, dyes, mica, fabric hueing agents, dye transfer inhibiting agents, chelating agents, suds suppressors, anti-foams, fabric softeners, perfumes, solvents, stabilizers, antimicrobial agents, neutralizers, and mixtures thereof.

19. The consumer product composition of claim 1, wherein the benefit active precursor is an oxyhalite.

20. The consumer product composition of claim 1, wherein the benefit active precursor is selected from the group consisting of chlorite salts, chlorate salts, bromite salts, bromate salts, iodite salts, iodate salts, and mixtures thereof.

21. The consumer product composition of claim 1, wherein the benefit active precursor is selected from the group consisting of chlorite salts, chlorate salts, and mixtures thereof.

22. The consumer product composition of claim 1, wherein the benefit active precursor is selected from the group consisting of sodium chlorite, sodium bromite sodium iodite, potassium chlorite, potassium bromite, potassium iodite, sodium chlorate, sodium bromate, sodium iodate, potassium chlorate, potassium bromate, potassium iodate, sodium hypochlorite, sodium hypobromite, sodium hypoiodite, sodium perchlorate, potassium perchlorate, and mixtures thereof.

23. The consumer product composition of claim 1, wherein the benefit active precursor is sodium chlorite.

24. The consumer product composition of claim 1, wherein the consumer product composition further comprises an non-photocatalyzable adjunct ingredient selected from the group consisting of nonionic surfactants, cationic surfactants, zwitterionic surfactants, amphoteric surfactants, builders, structurants, thickeners, clay soil removal agents, anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, mica, fabric hueing agents, dye transfer inhibiting agents, chelating agents, suds suppressors, anti-foams, fabric softeners, perfumes, solvents, stabilizers, antimicrobial agents, neutralizers and mixtures thereof.

25. The consumer product composition of claim 6, wherein the water soluble organic photoactivator comprises a photoactive moiety is selected from the group consisting of 1,1′-biphenyl-4,4′-diamine, 1,1′-biphenyl-4-amine, benzophenone, 1,1′-biphenyl-4,4′-diol, 1,1′-biphenyl-4-amine, 1,1′-biphenyl-4-ol, 1,1′:2′,1″-terphenyl, 1,1′:3′,1″-terphenyl, 1,1′:4′,1″:4″,1′″-quaterphenyl, 1,1′:4′,1″-terphenyl, 1,10-phenanthroline, 1,1′-biphenyl, 1,2,3,4-dibenzanthracene, 1,2-benzenedicarbonitrile, 1,3-isobenzofurandione, 1,4-naphthoquinone, 1,5-naphthalenediol, 10H-phenothiazine, 10H-phenoxazine, 10-methylacridone, 1-acetonaphthone, 1-chloroanthraquinone, 1-hydroxyanthraquinone, 1-naphthalenecarbonitrile, 1-naphthalenecarboxaldehyde, 1-naphthalenesulfonic acid, 1-naphthalenol, 2(1H)-quinolinone, 2,2′-biquinoline, 2,3-naphthalenediol, 2,6-dichlorobenzaldehyde, 21H,23H-porphine, 2-aminoanthraquinone, 2-benzoylthiophene, 2-chlorobenzaldehyde, 2-chlorothioxanthone, 2-ethylanthraquinone, 2H-1-benzopyran-2-one, 2-methoxythioxanthone, 2-methyl-1,4-naphthoquinone, 2-methyl-9(10-methyl)-acridinone, 2-methylanthraquinone, 2-methylbenzophenone, 2-naphthalenamine, 2-naphthalenecarboxylic acid, 2-naphthalenol, 2-nitro-9(10-methyl)-acridinone, 9(10-ethyl)-acridinone, 3,6-qcridinediamine, 3,9-dibromoperylene, 3,9-dicyanophenanthrene, 3-benzoylcoumarin, 3-methoxy-9-cyanophenanthrene, 3-methoxythioxanthone, 3′-methylacetophenone, 4,4′-dichlorobenzophenone, 4,4′-dimethoxybenzophenone, 4-bromobenzophenone, 4-chlorobenzophenone, 4′-fluoroacetophenone, 4-methoxybenzophenone, 4′-methylacetophenone, 4-methylbenzaldehyde, 4-methylbenzophenone, 4-phenylbenzophenone, 6-methylchromanone, 7-(diethylamino)coumarin, 7H-benz[de]anthracen-7-one, 7H-benzo[c]xanthen-7-one, 7H-furo[3,2-g][1]benzopyran-7-one, 9(10H)-acridinone, 9(10H)-anthracenone, 9(10-methyl)-acridinone, 9(10-phenyl)-acridinon, 9,10-anthracenedione, 9-acridinamine, 9-cyanophenanthrene, 9-fluorenone, 9H-carbazole, 9H-fluoren-2-amine, 9H-fluorene, 9H-thioxanthen-9-ol, 9H-thioxanthen-9-one, 9H-thioxanthene-2,9-diol, 9H-xanthen-9-one, acetophenone, acridene, acridine, acridone, anthracene, anthraquinone, anthrone, α-tetralone, benz[a]anthracene, benzaldehyde, benzamide, benzo[a]coronene, benzo[a]pyrene, benzo[f]quinoline, benzo[ghi]perylene, benzo[rst]pentaphene, benzophenone, benzoquinone, 2,3,5,6-tetramethyl, chrysene, coronene, dibenz[a,h]anthracene, dibenzo[b,def]chrysene, dibenzo[c,g]phenanthrene, dibenzo[def,mno]chrysene, dibenzo[def,p]chrysene, DL-tryptophan, fluoranthene, fluoren-9-one, fluorenone, isoquinoline, methoxycoumarin, methylacridone, michler's ketone, naphthacene, naphtho[1,2-g]chrysene, N-methylacridone, p-benzoquinone, p-benzoquinone, 2,3,5,6-tetrachloro, pentacene, phenanthrene, phenanthrenequinone, phenanthridine, phenanthro[3,4-c]phenanthrene, phenazine, phenothiazine, p-methoxyacetophenone, pyranthrene, pyrene, quinoline, quinoxaline, riboflavin 5′-(dihydrogen phosphate), thioxanthone, thymidine, xanthen-9-one, xanthone, and mixtures thereof.

26. The consumer product composition of claim 6, wherein the water soluble organic photoactivator comprises a photoactive moiety selected from the group consisting of xanthone, xanthene, thioxanthone, thioxanthene, phenothiazine, fluorescein, benzophenone, alloxazine, isoalloxazine, flavin, and mixtures thereof.

27. The consumer product composition of claim 6, wherein the photoactive moiety is thioxanthone.

28. The consumer product composition of claim 6, wherein the water soluble organic photoactivator comprises a hydrophilic moiety selected from the group consisting of alkylene oxide oligimers, alkylene oxide polymers, alkylene oxide copolymers, ethylene glycol, vinyl alcohol, vinyl pyrrolidone, acrylic acid, methacrylic acid, acrylamide, cellulose, carboxymethyl cellulose, chitosan, dextran, polysaccharides, 2-ethyl-2-oxazoline, hydroxyethyl methacrylate, vinyl pyridine-N-oxide, diallyl dimethyl ammonium chloride, maleic acid, lysine, isopropyl acrylamide, styrene sulfonic acid, vinyl methyl ether, vinyl phosphoinic acid, ethylene imine, and mixtures thereof.

29. The consumer product composition of claim 6, further comprising an adjunct ingredient selected from the group consisting of nonionic surfactants, cationic surfactants, zwitterionic surfactants, amphoteric surfactants, builders, structurants, thickeners, clay soil removal agents, anti-redeposition agents, polymeric soil release agents, polymeric dispersing agents, polymeric grease consumer product agents, enzymes, enzyme stabilizing systems, bleaching compounds, bleaching agents, bleach activators, bleach catalysts, brighteners, dyes, mica, fabric hueing agents, dye transfer inhibiting agents, chelating agents, suds suppressors, anti-foams, fabric softeners, perfumes, solvents, stabilizers, antimicrobial agents, neutralizers, and mixtures thereof.

30. The consumer product composition of claim 6, wherein the benefit active precursor is an oxyhalite.

31. The consumer product composition of claim 6, wherein the benefit active precursor is selected from the group consisting of chlorite salts, chlorate salts, bromite salts, bromate salts, iodite salts, iodate salts, and mixtures thereof.

32. The consumer product composition of claim 6, wherein the benefit active precursor is selected from the group consisting of chlorite salts, chlorate salts, and mixtures thereof.

33. The consumer product composition of claim 6, wherein the benefit active precursor is selected from the group consisting of sodium chlorite, sodium bromite sodium iodite, potassium chlorite, potassium bromite, potassium iodite, sodium chlorate, sodium bromate, sodium iodate, potassium chlorate, potassium bromate, potassium iodate, sodium hypochlorite, sodium hypobromite, sodium hypoiodite, sodium perchlorate, potassium perchlorate, and mixtures thereof.

34. The consumer product composition of claim 6, wherein the benefit active precursor is sodium chlorite.

35. The consumer product composition of claim 6, wherein the consumer product composition further comprises an non-photocatalyzable adjunct ingredient selected from the group consisting of nonionic surfactants, cationic surfactants, zwitterionic surfactants, amphoteric surfactants, builders, structurants, thickeners, clay soil removal agents, anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, mica, fabric hueing agents, dye transfer inhibiting agents, chelating agents, suds suppressors, anti-foams, fabric softeners, perfumes, solvents, stabilizers, antimicrobial agents, neutralizers and mixtures thereof.

36. A method of making a benefit active, the method comprising the step of exposing a consumer product composition according to claim 1 to light.

37. A method of cleaning a surface, the method comprising the steps of:
contacting the surface with a consumer product composition according to claim 1; and
exposing the consumer product composition to light.

38. A method of bleaching a stain, the method comprising the steps of:
contacting the stain with a consumer product composition according to claim 1; and
exposing the consumer product composition to light.

39. A method of disinfecting a surface, the method comprising the steps of:
contacting the surface with a consumer product composition according to claim 1; and
exposing the consumer product composition to light.

40. A method of removing biofilm from a surface, the method comprising the steps of contacting the biofilm with a consumer product composition according to claim 1; and
exposing the consumer product composition to light.

41. A method of cleaning a surface, wherein the method comprises the steps of:
dissolving the consumer product composition of claim 6 in water to form an aqueous solution comprising an electron acceptor;
contacting the surface or stain with the aqueous solution; and
exposing the aqueous solution to light.

42. The method of claim 41, wherein the electron acceptor is diatomic oxygen.
